(19) United States
(12) Patent Application Publication
(10) Pub. No.: US 2010/0092479 A1
(43) Pub. Date: Apr. 15, 2010

(54) Compositions and methods for treatment of viral diseases

(75) Inventors: Lisa M. Johansen, Belmont, MA (US); Christopher M. Owens, Cambridge, MA (US)

Correspondence Address: Gearhart Law LLC, 4 Femdale Avenue, Chatham, NJ, 07928, US

(73) Assignee: CombinatoRx (Singapore) Pte. Ltd., Singapore (SG)

(21) Appl. No.: 12/583,242

(22) Filed: Aug. 17, 2009

Related U.S. Application Data
(60) Provisional application No. 61/089,860, filed on Aug. 18, 2008.

(51) Int. Cl. A61K 39/395; A61K 31/66; A61K 31/335; A61K 31/496; A61K 31/135; A61K 31/56; A61K 31/397; A61K 31/351; A61P 35/00; A61P 31/12; A61P 9/10

(52) U.S. Cl. 424/141.1; 514/103; 514/450; 514/254.07; 514/654; 514/171; 514/647; 514/210.02; 514/460

(57) ABSTRACT

The present invention features compositions, methods, and kits useful in the treatment of viral diseases. In certain embodiments, the viral disease is caused by a single stranded RNA virus, a flaviviridae virus, or a hepatic virus. In particular embodiments, the viral disease is viral hepatitis (e.g., hepatitis A, hepatitis B, hepatitis C, hepatitis D, hepatitis E).

### CLAIM OF PRIORITY

This application claims priority to U.S. Ser. No. 61/089,860 filed Aug. 18, 2008, the contents of which are fully incorporated herein by reference.

### BACKGROUND OF THE INVENTION

The invention relates to the treatment of diseases caused by a virus. Diseases caused by viruses are major health problems worldwide, and include many potentially fatal or disabilitating illnesses. Viral diseases include diseases caused by single stranded RNA viruses, flaviviridae viruses, and hepatic viruses. In one example, viral hepatitis (e.g., hepatitis A, hepatitis B, hepatitis C, hepatitis D, and hepatitis E) can result in chronic or acute hepatitis. While vaccines protective against hepatitis A and hepatitis B exist, no cures for many viruses, including hepatitis B, C, D, or E, are available.

With regard to the hepatitis C virus (HCV), the Center for Disease Control estimates that 4.1 million Americans (1.6%) have been infected with this virus. Of those infected, 3.2 million are chronically infected, and HCV is the leading cause of death from liver disease in the United States. Hepatitis C is a major risk factor for developing liver cirrhosis and hepatocellular carcinoma, and the World Health Organization indicates that hepatitis C is responsible for two thirds of liver transplants. Worldwide, an estimated 180 million people, or about 3% of the world's population, are infected with HCV. No vaccine for hepatitis C is presently available, and the currently recommended therapy, a combination of pegylated interferon and ribavirin, is effective in only about 50% of those infected with HCV genotype 1. Further, both interferon and ribavirin have potentially serious side effects, which include seizures, acute heart or kidney failure, and anemia.

Given the lack of safe, efficacious treatments for many viral diseases, there exists a need for improved therapies.

### SUMMARY OF THE INVENTION

Based on the results of our screen identifying compounds and combinations of compounds having antiviral activity, the present invention features compositions, methods, and kits for the treatment of viral disease (e.g., caused by the viruses described herein). In certain embodiments, the viral disease may be caused by a virus that is a member of one or more of the following groups: single stranded RNA viruses, flaviviridae viruses (e.g., a hepacivirus such as HCV, flavivirus, pestivirus, or hepatitis G virus), and hepatic viruses. HCV, for example, is a single stranded RNA virus, a flaviviridae virus, and a hepatic virus. In certain embodiments, the viral disease is caused by the hepatitis C virus. Additional exemplary viruses are described herein.

Accordingly in a first aspect, the invention features a composition containing (a) an inhibitor of a cholesterol biosynthetic enzyme and (b) sertraline, UK-416244, or an analog thereof. The cholesterol biosynthesis inhibitor may inhibit, for example, HMG-CoA synthase, mevalonate kinase, phosphomevalonate kinase, farnesyl transferase, geranylgeranyl transferase, farnesyl diphosphate synthase synthase, squalene synthase, squalene monooxygenase, lanosterol synthase, lanosterol 14α-demethylase, Δ14-sterol reductase, C-4 methyl sterol oxidase, 3β-hydroxysteroid dehydrogenase, 3-ketosteroid dehydrogenase, sterol Δ8,Δ7 isomerase, sterol-C5-desaturase, sterol Δ7 reductase, or sterol Δ24 reductase. In one embodiment, the inhibitor is not amorolfine. In certain embodiments, the inhibitor, e.g., fenpropimorph, may inhibit more than one cholesterol biosynthetic enzyme. In a preferred embodiment, the composition contains sertraline and a cholesterol biosynthesis inhibitor selected from the group Ro 48-8071, fenpropimorph, BIBB-515, clomiphene, farnesol, triparanol, terconazole, AY-9944, and alendronate.

In another aspect, the invention features a composition including a pair of agents, both of which inhibit a cholesterol biosynthetic enzyme. In certain embodiments, the agents of the pair, e.g., colestolone and simvastatin, inhibit the same enzyme. In other embodiments, the agents of the pair, e.g., clomiphene and Ro 48-8071, inhibit different enzymes. In yet another embodiment, one agent of a pair, e.g., fenpropimorph, inhibits more than one cholesterol biosynthetic enzyme. In certain embodiments, the composition contains a pair of agents selected from the group consisting of AY-9944 and amorolfine; colestolone and simvastatin; BIBB-515 and colestolone; AY-9944 and fenpropimorph; clomiphene and fenpropimorph; clomiphene and Ro 48-8071; amorolfine and GGTI-286; alendronate and colestolone; colestolone and fenpropimorph; amorolfine and terconzaole; amorolfine and clomiphene; fenpropimorph and triparanol; colestolone and SR12813; colestolone and Ro 48-8071; clomiphene and terconazole; GGTI-286 and colestolone; and GGTI-286 and Ro 48-8071.

In another aspect, the invention features a composition that includes (a) an inhibitor of cholesterol biosynthesis and (b) an inhibitor of cholesterol absorption. In one embodiment, the inhibitor of cholesterol biosynthesis is not amorolfine. In one embodiment, the cholesterol absorption inhibitor is ezetimibe. In other embodiments, the composition contains fenpropimorph, AY-9944, or colestolone.

In another aspect, the invention features a composition that includes (a) an inhibitor of cholesterol biosynthesis and (b) an inhibitor of sphingomyelin biosynthesis. In certain embodiments, the sphingomyelin biosynthesis inhibitor inhibits Acetyl-CoA carboxylase or serine palmitoyl transferase. In one embodiment, the composition contains a pair of agents selected from colestolone and TOFA; amorolfine and TOFA; and BIBB-515 and TOFA.

In another aspect, the invention features a composition including (a) an inhibitor of a sphingomyelin biosynthetic enzyme and (b) sertraline, a sertraline analog, UK-416244, or a UK-416244 analog. In one embodiment, the composition contains myriocin and sertraline.

In another aspect, the invention features a composition comprising one or more agents whose target proteins is selected from Acetyl-CoA carboxylase (ACoAC), Sterol regulatory element binding protein (SREBP), 3-hydroxy-3-methylglutaryl-Coenzyme A reductase (HMGCR), Farnesyl pyrophosphate synthase (FPPS), Squalene Synthase (SQLS), Oxidosqualene cyclase (OSC), Lanosterol C14-demethylase (C14dM), Sterol delta-7 and delta-14 reductase (d7/d14R), and protein geranylgeranyl transferase I (PGGT). In another aspect, the invention features a composition that includes the compound know as U18666A.

In another aspect, the invention features a composition comprising one or more pairs of agents. These pairs are selected from cholestolonene and TOFA, cholestolone and SR-12813, cholestolone and simvastatin, cholestolone and alendronate, cholestolone and farnesol, cholestolone and squalestatin, cholestolone and clomiphene, cholestolone and R048-8071, cholestolone and U18666A, cholestolonene and terconazole, cholestolone and amorolfine, cholestolone and fenpropimorph, cholestolone and AY-9944, and cholestolone and triparanol.

In another aspect, the invention features a composition comprising one or more pairs of agents. These pairs are selected from SR-12813 and TOFA, SR-12813 and cholestolone, SR-12813 and simvastatin, SR-12813 and alendronate, SR-12813 and farnesol, SR-12813 and squalestatin, SR-12813 and clomiphene, SR-12813 and R048-8071, SR-12813 and U18666A, SR-12813 and terconazole, SR-12813 and amorolfine, SR-12813 and fenpropimorph, SR-12813 and AY-9944, and SR-12813 and triparanol.

In another aspect, the invention features a composition comprising one or more pairs of agents. These pairs are selected from simvastatin and TOFA, simvastatin and colestolone, simvastatin and SR-12813, simvastatin and alendronate, simvastatin and farnesol, simvastatin and squalestatin, simvastatin and clomiphene, simvastatin and R048-8071, simvastatin and U18666A, simvastatin and terconazole, simvastatin and amorolifene, simvastatin and fenpropimorph, simvastatin and AY-9944, and simvastatin and triparanol.

In another aspect, the invention features a composition comprising one or more pairs of agents. These pairs are selected from alendronate and TOFA, alendronate and cholestolone, alendronate and SR-12813, alendronate and simvastatin, alendronate and farnesol, alendronate and squalestatin, alendronate and clomiphene, alendronate and R048-8071, alendronate and U18666A, alendronate and terconazole, alendronate and amorolfine, alendronate and fenpropimorph, alendronate and AY-9944, and alendronate and triparanol.

In another aspect, the invention features a composition comprising one or more pairs of agents. These pairs are selected from farnesol and TOFA, farnesol and cholestolone, farnesol and SR-12813, farnesol and simvastatin, farnesol and alendronate, farnesol and squalestatin, farnesol and clomiphene, farnesol and R048-8071, farnesol and U18666A, farnesol and terconazole, farnesol and amorolfine, farnesol and fenpropimorph, farnesol and AY-9944, and farnesol and triparanol.

In another aspect, the invention features a composition comprising one or more pairs of agents. These pairs are selected from squalestatin and TOFA, squalestatin and cholestolone, squalestatin and SR-12813, squalestatin and simvastatin, squalestatin and alendronate, squalestatin and farnesol, squalestatin and clomiphene, squalestatin and R048-8071, squalestatin and U18666A, squalestatin and terconazole, squalestatin and amorolfine, squalestatin and fenpropimorph, squalestatin and AY-9944, and squalestatin and triparanol.

In another aspect, the invention features a composition comprising one or more pairs of agents. These pairs are selected from clomiphene and TOFA, clomiphene and cholestolone, clomiphene and SR-12813, clomiphene and simvastatin, clomiphene and alendronate, clomiphene and farnesol, clomiphene and squalestatin, clomiphene and R048-8071, clomiphene and U18666A, clomiphene and terconazole, clomiphene and amorolfine, clomiphene and fenpropimorph, clomiphene and AY-9944, and clomiphene and triparanol.

In another aspect, the invention features a composition comprising one or more pairs of agents. These pairs are selected from R048-8071 and TOFA, R048-8071 and cholestolone, R048-8071 and SR-12813, R048-8071 and simvastatin, R048-8071 and alendronate, R048-8071 and farnesol, R048-8071 and squalestatin, R048-8071 and clomiphene, R048-8071 and U18666A, R048-8071 and terconazole, R048-8071 and amorolfine, R048-8071 and fenpropimorph, R048-8071 and AY-9944, and R048-8071 and triparanol.

In another aspect, the invention features a composition comprising one or more pairs of agents. These pairs are selected from U18666A and TOFA, U18666A and cholestolone, U18666A and SR-12813, U18666A and simvastatin, U18666A and alendronate, U18666A and farnesol, U18666A and squalestatin, U18666A and clomiphene, U18666A and R048-8071, U18666A and terconazole, U18666A and amorolfine, U18666A and fenpropimorph, U18666A and AY-9944, and U18666A and triparanol.

In another aspect, the invention features a composition comprising one or more pairs of agents. These pairs are selected from terconazole and TOFA, terconazole and cholestolone, terconazole and SR-12813, terconazole and simvastatin, terconazole and alendronate, terconazole and farnesol, terconazole and squalestatin, terconazole and clomiphene, terconazole and R048-8071, terconazole and U18666A, terconazole and amorolfine, terconazole and fenpropimorph, terconazole and AY-9944, and terconazole and triparanol.

In another aspect, the invention features a composition comprising one or more pairs of agents. These pairs are selected from amorolfine and TOFA, amorolfine and cholestolone, amorolfine and SR-12813, amorolfine and simvastatin, amorolfine and alendronate, amorolfine and farnesol, amorolfine and squalestatin, amorolfine and clomiphene, amorolfine and R048-8071, amorolfine and U18666A, amorolfine and terconazole, amorolfine and fenpropimorph, amorolfine and AY-9944, and amorolfine and triparanol.

In another aspect, the invention features a composition comprising one or more pairs of agents. These pairs are selected from fenpropimorph and TOFA, fenpropimorph and cholestolone, fenpropimorph and SR-12813, fenpropimorph and simvastatin, fenpropimorph and alendronate, fenpropimorph and farnesol, fenpropimorph and squalestatin, fenpropimorph and clomiphene, fenpropimorph and R048-8071; fenpropimorph and U18666A, fenpropimorph and terconazole, fenpropimorph and amorolfine, fenpropimorph and AY-9944, and fenpropimorph and triparanol.

In another aspect, the invention features a composition comprising one or more pairs of agents. These pairs are selected from AY-9944 and TOFA, AY-9944 and cholestolone, AY-9944 and SR-12813, AY-9944 and simvastatin, AY-9944 and alendronate, AY-9944 and farnesol, AY-9944 and squalestatin, AY-9944 and clomiphene, AY-9944 and R048-8071, AY-9944 and U18666A, AY-9944 and terconazole, AY-9944 and amorolfine, AY-9944 and fenpropimorph, and AY-9944 and triparanol.

In another aspect, the invention features a composition comprising one or more pairs of agents. These pairs are selected from triparanol and TOFA, triparanol and cholestolone, triparanol and SR-12813, triparanol and simvastatin, triparanol and alendronate, triparanol and farnesol, triparanol and squalestatin, triparanol and clomiphene, triparanol and R048-8071, triparanol and U18666A, triparanol and terconazole, triparanol and amorolfine, triparanol and fenpropimorph, and triparanol and AY-9944.

In another aspect, the invention features a composition comprising one or more pairs of agents. These pairs are selected from GGTI-286 and TOFA, GGTI-286 and cholestolonene, GGTI-286 and SR-12813, GGTI-286 and simvastatin, GGTI-286 and alendronate, GGTI-286 and farnesol, GGTI-286 and squalestatin, GGTI-286 and clomiphene, GGTI-286 and R048-8071, GGTI-286 and U18666A, GGTI-286and terconazole, GGTI-286 and amorolfine, GGTI-286 and fenpropimorph, GGTI-286 and AY-9944, and GGTI-286 and triparanol.

In another aspect, the invention features a composition wherein the combined activity of the one or more pairs of agents is synergistic in inhibiting HCV replicon. In another aspect, the invention features a composition wherein the combined activity of the one or more pairs of agents targets sterol enzyme pathways downstream of OSC. In another aspect, the invention features a composition wherein the combined activity of the one or more pairs of agents targets sterol enzyme pathways upstream of OSC. In another aspect, the invention features a composition wherein the combined activity of the one or more pairs of agents targets sterol enzyme pathways downstream of OSC and upstream of OSI.

In another aspect, the invention features a composition wherein the combined activity of the one or more pairs of agents is toxic to the virus and has little or no toxicity in host cells.

In certain embodiments, the compositions of the invention may contain a pair of agents selected from Table 1.

TABLE 1
Pairs of agents

| Compound A | Compound B |
|---|---|
| Ro 48-8071 | Sertraline Hydrochloride |
| Fenpropimorph | Sertraline Hydrochloride |
| BIBB-515 | Sertraline Hydrochloride |
| Clomiphene Citrate | Sertraline Hydrochloride |
| AY-9944 | Amorolfine Hydrochloride |
| Farnesol | Sertraline Hydrochloride |
| Colestolone | TOFA |
| Triparanol | Sertraline Hydrochloride |
| Colestolone | Simvastatin |
| Terconazole | Sertraline Hydrochloride |
| Fenpropimorph | Ezetimibe |
| AY-9944 | Sertraline Hydrochloride |
| BIBB-515 | Colestolone |
| AY-9944 | Fenpropimorph |
| Alendronate Sodium | Sertraline Hydrochloride |
| Clomiphene Citrate | Fenpropimorph |
| Clomiphene Citrate | Ro 48-8071 |
| Myriocin | Sertraline Hydrochloride |
| Amorolfine Hydrochloride | GGTI-286 |
| Alendronate Sodium | Colestolone |
| Colestolone | Fenpropimorph |
| Amorolfine Hydrochloride | TOFA |
| Amorolfine Hydrochloride | Terconazole |
| Amorolfine Hydrochloride | Clomiphene Citrate |
| BIBB-515 | TOFA |
| AY-9944 | Ezetimibe |
| SR12813 | Colestolone |
| Fenpropimorph | Triparanol |
| Colestolone | SR 12813 |
| Colestolone | Ro 48-8071 |
| Clomiphene | Terconazole |
| Colestolone | Ezetimibe |
| GGTI-286 | Colestolone |
| GGTI-286 | Ro 48-8071 |

In any of the above aspects, the two agents may be present in amounts that, when administered to a patient having a viral disease, e.g., any viral disease described herein, are effective to treat the patient. The composition may be formulated, for example, for oral, systemic, parenteral, topical (e.g., ophthalmic, dermatologic), intravenous, or intramuscular administration.

In another aspect, the invention features a method for treating a patient with a viral disease that includes administering to the patient an inhibitor of cholesterol biosynthesis or an inhibitor of sphingomyelin biosynthesis in an amount that is effective to treat the patient. In certain embodiments, the agent is selected from the group consisting of lovastatin, mevastatin, TOFA, terconazole, itavastatin, triparanol, clomiphene, AY-9944, colestolone, simvastatin, Ro 48-8071, fluvastatin, amorolfine, SR12813, BIBB-515, myriocin, and GGTI-286. In other embodiments, the agent is an analog of lovastatin, mevastatin, TOFA, terconazole, itavastatin, triparanol, clomiphene, AY-9944, colestolone, simvastatin, Ro 48-8071, fluvastatin, amorolfine, SR12813, BIBB-515, myriocin, or GGTI-286. In another aspect, the invention features a method that includes administering to a patient with a viral disease a pair of active agents in amounts that together are effective to treat the patient. The pairs of agents may consist of an inhibitor of cholesterol biosynthesis and sertraline, a sertraline analog, UK-416244, or a UK-416244 analog; two inhibitors of cholesterol biosynthesis; an inhibitor of cholesterol biosynthesis and an inhibitor of cholesterol absorption; an inhibitor of cholesterol biosynthesis and an inhibitor of sphingomyelin biosynthesis; or an inhibitor of sphingomyelin biosynthesis and sertraline, a sertraline analog, UK-416244, or a UK-416244 analog. In certain embodiments of this aspect, the pair of agents is selected from the pairs: Ro 48-8071 and sertraline; fenproprimorph and sertraline; BIBB-515 and sertraline; clomiphene and sertraline; farnesol and sertraline; triparanol and sertraline; terconazole and sertraline; AY-9944 and sertraline; alendronate and sertraline; AY-9944 and amorolfine; colestolone and simvastatin; BIBB-515 and colestolone; AY-9944 and fenpropimorph; clomiphene and fenpropimorph; clomiphene and Ro 48-8071; amorolfine and GGTI-286; alendronate and colestolone; colestolone and fenpropimorph; amorolfine and terconzaole; amorolfine and clomiphene; SR12813 and colestolone; fenpropimorph and triparanol; colestolone and Ro 48-8071; clomiphene and terconazole; GGTI-286 and colestolone; GGTI-286 and Ro 48-8071; fenpropimorph and ezetimibe; AY-9944 and ezetimibe; colestolone and ezetimibe; colestolone and TOFA; amorolfine and TOFA; BIBB-515 and TOFA; and myriocin and sertraline.

In another aspect, the invention features a method that includes administering to a patient with a viral disease a plurality of agents, where the agents are administered within 28 days (e.g., within 21, 14, 10, 7, 5, 4, 3, 2, or 1 days) or within 24 hours (e.g., 12, 6, 3, 2, or 1 hours; or concomitantly) of each other, in amounts that together are effective to treat the patient. The method may include administering a pair of active agents consisting of an inhibitor of cholesterol biosynthesis and sertraline, a sertraline analog, UK-416244, or a UK-416244 analog; a pair of inhibitors of cholesterol biosynthesis; an inhibitor of cholesterol biosynthesis and an inhibitor of cholesterol absorption; an inhibitor of cholesterol biosynthesis and an inhibitor of sphingomyelin biosynthesis; or an inhibitor of sphingomyelin biosynthesis and sertraline, a sertraline analog, UK-416244, or a UK-416244 analog. The methods that include administering to the patient a pair of active agents may be performed in conjunction with administering to the patient an additional treatment (e.g., an antiviral therapy such as those agents listed in Table 2 and Table 3) for a'viral disease, where the method and the additional treatment are administered within 6 months (e.g., within 3, 2, or 1 months; within 28, 21, 14, 10, 7, 5, 4, 3, 2, or 1 days; within 24, 12, 6, 3, 2, or 1 hours; or concomitantly) of each other.

The methods of the invention may include administering agents to the patient by intravenous, intramuscular, inhalation, topical (e.g., ophthalmic, determatologic), or oral administration.

In certain embodiments of any of the above methods (e.g., methods including administration of sertraline), the patient being treated has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder. In other embodiments, (e.g., methods including administration of an cholesterol biosynthesis inhibitor), the patient being treated has not been diagnosed with or does not suffer from hypercholesterolemia, primary familial hypercholesterolemia (heterozygous variant), mixed hyperlipidaemia (corresponding to type IIa and IIb of the Fredrickson classification), or coronary artery disease, or has not had a myocardial infarction, a cerebrovascular event, an coronary bypass surgery, or a translumen percutaneous coronary angioplasty.

In another aspect, the invention features a kit including a composition containing a pair of agents selected from any of the pairs of of Table 1; and instructions for administering the composition to a patient having a viral disease.

In another aspect, the invention features a kit including a composition including (i) a pair of agents from Table 1, (ii) one or more agents of Table 2 and Table 3; and instructions for administering the composition to a patient having a viral disease.

In another aspect, the invention features a kit including (a) a pair of agents from Table 1, (b) one or more agents of Table 2 and Table 3; and instructions for administering (a) and (b) to a patient having a viral disease.

In another aspect, the invention features a kit including an agent selected from lovastatin, mevastatin, TOFA, terconazole, itavastatin, clomiphene, colestolone, simvastatin, Ro 48-8071, fluvastatin, amorolfine, SR12813, BIBB-515, myriocin, and GGTI-286; and instructions for administering the agent to a patient having a viral disease.

In another aspect, the invention features a kit including an inhibitor of a cholesterol biosynthetic enzyme (e.g., Ro 48-8071, terconazole, or AY-9944); sertraline, a sertraline analog, UK-416244, or a UK-416244 analog; and instructions for administering the inhibitor of a cholesterol biosynthetic enzyme and the sertraline (sertraline analog, UK-416244, or a UK-416244 analog) to a patient having a viral disease.

In another aspect, the invention features a kit including a pair of inhibitors of cholesterol biosynthesis (e.g., AY-9944 and fenpropimorph; or colestolone and simvastatin); and instructions for administering the pair of agents to a patient having a viral disease.

In another aspect, the invention features a kit including an inhibitor of a cholesterol biosynthetic enzyme (e.g., fenpropimorph); and an inhibitor of cholesterol absorption (e.g., ezetimibe); and instructions for administering the inhibitor of a cholesterol biosynthetic enzyme and the inhibitor of cholesterol absorption to a patient having a viral disease.

In another aspect, the invention features a kit including an inhibitor of a cholesterol biosynthetic enzyme (e.g., BIBB-515); an inhibitor of a sphingomyelin synthetic enzyme (e.g., TOFA); and instructions for administering the inhibitor of a cholesterol biosynthetic enzyme and the inhibitor of a sphingomyelin synthetic enzyme to a patient having a viral disease.

In another aspect, the invention features a kit containing an inhibitor of a sphingomyelin biosynthetic enzyme (e.g., myriocin); sertraline, a sertraline analog, UK-416244, or a UK-416244 analog; and instructions for administering the inhibitor of a sphingomyelin biosynthetic enzyme and sertraline (or sertraline analog, UK-416244, or a UK-416244 analog) to a patient having a viral disease. In one embodiment, the invention features a kit including a pair of agents selected from the pairs including Ro 48-8071 and sertraline; fenpropimorph and sertraline; BIBB-515 and sertraline; clomiphene and sertraline; farnesol and sertraline; triparanol and sertraline; terconazole and sertraline; AY-9944 and sertraline; and alendronate and sertraline; AY-9944 and amorolfine; colestolone and simvastatin; BIBB-515 and colestolone; AY-9944 and fenpropimorph; clomiphene and fenpropimorph; clomiphene and Ro 48-8071; amorolfine and GGTI-286; alendronate and colestolone; colestolone and fenpropimorph; amorolfine and terconzaole; amorolfine and clomiphene; SR12813 and colestolone; fenpropimorph and triparanol; colestolone and Ro 48-8071; clomiphene and terconazole; GGTI-286 and colestolorie; GGTI-286 and Ro 48-8071; fenpropimorph and ezetimibe; AY-9944 and ezetimibe; colestolone and ezetimibe; colestolone and TOFA; amorolfine and TOFA; BIBB-515 and TOFA; and myriocin and sertraline.

In any of the aspects of the invention featuring a kit, the kit may contain instructions for administering the active agent(s) to a patient having hepatitis C.

The viral disease referred to in any of the above aspects of the invention, including the compositions, methods of treatment, and kits of the invention, may be caused by a single stranded RNA virus, a flaviviridae virus (e.g., a hepacivirus such as HCV, flavivirus, pestivirus, or hepatitis G virus), or a hepatic virus (e.g., any hepatic virus described herein such as hepatitis A, hepatitis B, hepatitis C, hepatitis D, hepatitis E, non-ABCDE hepatitis, or hepatitis G). In certain embodiments, the viral disease is caused by a flavivirus which include without limitation Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, royal farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, west Nile, Yaounde, yellow fever, and Zika viruses, or any of the viruses described in Chapter 31 of Fields Virology, Fields, B. N., Knipe, D. M., and Howley, P. M., eds. Lippincott-Raven Publishers, Philadelphia, Pa., 1996. In other embodiments, the viral disease is caused by a pestivirus, which include bovine viral diarrhea virus (“BVDV”), classical swine fever virus (“CSFV,” also called hog cholera virus), border disease virus (“BDV”) and any of those discussed in Chapter 33, of Fields Virology, supra. In other embodiments, the viral disease is caused by a virus such as hepatitis A, hepatitis B, hepatitis C (e.g., genotype 1 such as 1a or 1b; genotype 2 such as. 2a, 2b, or 2c; genotype 3; genotype 4; genotype 5; genotype 6); hepatitis D; or hepatitis E. The viral hepatitis may further be a non-ABCDE viral hepatitis (e.g., hepatitis G).

Additional viral therapies are listed in Table 2 and Table 3.

TABLE 2
Additional viral therapies

| | | | |
|---|---|---|---|
| (+)-Calanolide A | (+)-Dihydrocalanolide A | 145U87 | 2-Nor-cyclic GMP |
| 3,4-Dicaffeoylquinic acid | 3-Hydroxymethyl | 3-Hydroxyphthaloyl-beta- | 3-Nitrosobenzamide |
| | dicamphanoyl khellactone | lactoglobulin | |
| 4-Azidothymidine | 4-Methyl dicamphanoyl | 524C79 | 739W94 |
| | khellactone | | |
| A 160621 | A 315675 | A 315677 | A 5021 |
| A 74259 | A 74704 | A 77003 | A 80735 |
| A 80987 | A 91883A | A 98881 | Abacavir |
| AC 2 | Acemannan | Acetylcysteine-Zambon | ACH 126445 |
| ACH 126447 | Aciclovir (e.g., extended | Aciclovir-PMPA | ACP HIP |
| | release, controlled release, | | |
| | topical patch) | | |
| Actinohivin | AD 439 | AD 519 | Adamantylamide dipeptide |
| ADS J1 | Afovirsen | AG 1284 | AG 1350 |
| AG 1478 | AG 1859 | AG 555 | AG 6840 |
| AG 6863 | AGT-1 | AHA 008 | Aidfarel |
| AL 721 | Alamifovir | Albumin/interferon-alpha | ALN RSV01 |
| Alovudine | Alpha HGA | Alpha-IPDX | Alpha-antitrypsin |
| Alvircept sudotox | Alvocidib | ALX 0019 | ALX 404C |
| AM 285 | AM 365 | Amantadine | AMD 070 |
| AMD 3329 | AMD 3465 | AMD 8664 | Amdoxovir |
| Amidinomycin | Aminopeptidase | Amitivir | Ampligen |
| Amprenavir | AMZ 0026 | Ancriviroc | Anti-CCR5 monoclonal |
| | | | antibody |
| Anti-CCR5/CXCR4 sheep | Anti-CD3 monoclonal | Anti-CD4 monoclonal | Anti-CD7 monoclonal |
| monoclonal antibody | antibody CD4IgG conjugate | antibody | antibody |
| Anti-CD8 monoclonal antibody | Anti-CMV monoclonal | Anti-hepatitis B ribozyme | Anti-HIV catalytic antibody |
| | antibody | | |
| Anti-HIV immunotoxin (IVAX) | Anti-HIV-1 human | Anti-HIV-1 human | Anti-HIV-1 human |
| | monoclonal antibody 2F5 | monoclonal antibody 2G12 | monoclonal antibody 4E10 |
| Antineoplaston AS2 1 (e.g., oral) | Anti-RSV antibody (Intracel, | Antisense oligonucleotide PB2 | Aop-RANTES |
| | Corp.) | AUG | |
| Aplaviroc | Apricitabine | AQ 148 | AR 132 |
| AR 177 | ARB 95214 | ARB 97265 | ARB 97268 |
| | ARQ 323 | AS 101 | AT 61 |
| Atazanavir | Atevirdine | AV 1101 | AV 2921 |
| AV 2923 | AV 2925 | AV 2927 | Avarol |
| AXD 455 | Azidodideoxyguanosine | Azodicarbonamide | Bafilomycin A1 |
| Baicalin | BAY 414109 | BAY 439695 | BAY 504798 |
| BAY Z 4305 | BB 10010 | BB 2116 | BCH 10652 |
| BCH 371 | BCH 527 | BCTP | BCX 140 |
| BCX 1591 | BCX 1827 | BCX 1898 | BCX 1923 |
| BEA | BEA 005 | Bellenamine | Benanomicin A |
| Benzalkonium (e.g., chloride) | Benzalkonium | Beta-D-FDOC | Beta-L-ddC |
| | chloride/octoxynol 9 (e.g., | | |
| | vaginal gel) | | |
| Beta-L-FddC | Bevirimat | BG 777 | BGP 15 |
| BILA 2185 BS | BILR 355 | BIRM ECA 10-142 | BL 1743 |
| BM 510836 | BMS 181167-02 | BMS 181184 | BMS 182193 |
| BMS 186318 | BMS 187071 | BMS 488043 | BMS 806 |
| BMY 27709 | Brecanavir | Brefeldin A | Brequinar |
| Brivudine | BRL 47923DP | BSL 4 | BST 5001 |
| BTA 188 | BTA 798 | C 1605 | C 2507 |
| C31G | Calcium spirulan | Canventol | Capravirine |
| Carbendazim | Carbocyclic deazaadenosine | Carbopol polymer gel | Carbovir |
| CC 3052 | CD4 fusion toxin | CD4 IgG | CD4-ricin chain A |
| Cellulose sulfate | CF 1743 | CFY 196 | CGA 137053 |
| CGP 35269 | CGP 49689 | CGP 53437 | CGP 53820 |
| CGP 57813 | CGP 61783 | CGP 64222 | CGP 70726 |
| CGP 75136 | CGP 75176 | CGP 75355 | CI 1012 |
| CI 1013 | Cidofovir | Civamide | CL 190038 |
| CL 387626 | Clevudine | CMV 423 | CMX 001 |
| CNBA-Na | CNJ 102 | Cobra venom peptide | Conocurvone |
| Cosalane | Costatolide | CP 1018161 | CP 38 |
| CP 51 | CPFDD | CRL 1072 | Crofelemer |
| CS 8958 | CS 92 | CT 2576 | CTC 96 |
| Curdlan sulfate | Cyanovirin-N | CYT 99007 | Cytomegalovirus immune |
| | | | globulin |
| DAB486interleukin-2 | DABO 1220 | Dacopafant | DAP 30 |
| DAP 32 | Dapivirine | Darunavir | D-aspartic-beta-hydroxamate |
| DB 340 | DDCDP-DG | DDGA | Deazaadenosine |
| Deazaneplanocin A | DEB 025 | Delavirdine | Delmitide |
| Denileukin diftitox | Deoxyfluoroguanosine | DES 6 | Dexelvucitabine |
| Dextran sulfate | Dextrin 2-sulfate | DG 35 | Didanosine |
| Dideoxyadenosine | Dideoxyguanosine | Dideoxythymidine | Didox |
| Dihydrocostatolide | Dinitrochlorobenzene | DL 110 | DMP 323 |
| DMP 850 | DMP 851 | DmTr-ODN12 | Docosanol |
| DP 107 | DPC 082 | DPC 083 | DPC 681 |
| DPC 684 | DPC 961 | DPC 963 | Droxinavir |
| DUP 925 | DYE | E 913 | EB-Foscarnet |
| E-EPSEU | EGS 21 | EHT 899 | Elvucitabine |
| EM 1421 | EM 2487 | Emivirine | Emtricitabine |
| Emtricitabine/tenofovir | Enfuvirtide | Entecavir | Eosinophil-derived |
| disoproxil fumarate | | | neutralizing agent |
| Episiastatin B | ET 007 | Etanercept | Ether lipid analogue |
| Etoviram | Etravirine | F 105 | F 36 |
| F 50003 | Famciclovir | Fasudil | Fattiviracin A1 |
| FEAU | Feglymycin | Felvizumab | FGI 345 |
| Fiacitabine | Fialuridine | FLG | Flutimide |
| Fomivirsen | Fosalvudine tidoxil | Fosamprenavir | Foscarnet Sodium |
| Fozivudine | FP 21399 | F-PBT | FPMPA |
| FPMPDAP | FR 191512 | FR 198248 | Galactan sulfate |
| Ganciclovir | GAP 31 | GCA 186 | GCPK |
| GE 20372A | GE 20372B | GEM 122 | GEM 132 |
| GEM 144 | GEM 92 | GEM 93 | Glamolec |
| Glutathionarsenoxide | Glycovir | GMDP | GO 6976 |
| GO 7716 | GO 7775 | Gossypol | GPG-NH2 |
| GPI 1485 | GPI 2A | GPs 0193 | GR 137615 |
| GR 92938X | GS 2838 | GS 2992 | GS 3333 |
| GS 3435 | GS 4071 | GS 438 | GS 7340 |
| GS 9005 | GS 9160 | GS 930 | GW 275175 |
| GW 5950X | HB 19 | HBY 946 | HE 317 |
| Hepatitis B immune globulin | HEPT | HGS-H/A27 | HI 236 |
| HI 240 | HI 244 | HI 280 | HI 346 |
| HI 443 | HI 445 | HIV DNA vaccine (Antigen | Thiovir |
| | | Express, Inc.) | |
| HIV immune globulin | HIV immune plasma | HL 9 | HOE BAY 793 |
| HRG 214 | HS 058 | Hydroxycarbamide | Hydroxychloroquine |
| I 152 | IAZT | Idoxuridine | IM28 |
| ImmStat | ImmuDyn | Immunocal | Imreg 1 |
| Incadronic acid | INCB 9471 | Indinavir | Infliximab |
| Influenza matrix protein Zn2+ | Ingenol Triacetate | Inophyllum B | Inosine pranobex |
| finger peptide | | | |
| Interferon-tau | Interleukin-1 receptor type 1 | Interleukin-13 | Interleukin-15 |
| Interleukin-16 | Interleukin-2 agonist | Interleukin-4 | IPdR |
| Ipilimumab | ISIS 13312 | Iso ddA | ITI 002 |
| ITI 011 | JBP 485 | JCA 304 | JE 2147 |
| JM 1596 | JM 2763 | JTK 303 | K 12 |
| K 37 | K 42 | Kamizol | kethoxal |
| Kijimicin | Kistamicin | KKKI 538 | KM 043 |
| KNI 102 | KNI 241 | KNI 272 | KNI 413 |
| KNI 684 | Kootikuppala | KP 1461 | KPC 2 |
| KRH 1120 | L 689502 | L 693549 | L 696229 |
| L 696474 | L 696661 | L 697639 | L 697661 |
| L 708906 | L 731988 | L 732801 | L 734005 |
| L 735882 | L 738372 | L 738684 | L 738872 |
| L 739594 | L 748496 | L 754394 | L 756423 |
| L 870810 | L HSA ara AMP | Lamivudine/abacavir | Lamivudine/zidovudine |
| Lamivudine/zidovudine/abacavir | Lasinavir | LB 71116 | LB 71148 |
| LB 71262 | LB 71350 | LB 80380 | L-chicoric acid |
| Lecithinized superoxide | Leflunomide | Lentinan | Leukocyte interleukin injection |
| dismutase | | | (CEL-SCI Corp.) |
| Leukotriene B4-LTB4 | Levcycloserine | Levofloxacin | Lexithromycin |
| Liposomal ODG-PFA-OMe | Lithium succinate | Lobucavir | Lodenosine |
| Lopinavir | Loviride | Lufironil | LY 180299 |
| LY 214624 | LY 253963 | LY 289612 | LY 296242 |
| LY 296416 | LY 309391 | LY 309840 | LY 311912 |
| LY 314163 | LY 314177 | LY 316683 | LY 326188 |
| LY 326594 | LY 326620 | LY 338387 | LY 343814 |
| LY 354400 | LY 355455 | LY 366094 | LY 366405 |
| LY 368177 | LY 73497 | Lysozyme | M 40401 |
| M4N | Madu | Mannan sulfate | MAP 30 |
| Maraviroc | Maribavir | Masoprocol | MB-Foscarnet |
| MC 207044 | MC 207685 | MC 867 | mCDS71 |
| MDI-P | MDL 101028 | MDL 20610 | MDL 27393 |
| MDL 73669 | MDL 74428 | MDL 74695 | MDL 74968 |
| MDX 240 | ME 609 | MEDI 488 | MEN 10690 |
| MEN 10979 | MER N5075A | Met-enkephalin | Methisazone |
| MGN 3 | Michellamine B | Miglustat | MIV 150 |
| MIV 210 | Mivotilate | MK 0518 | MK 944A |
| MM 1 | MMS 1 | MOL 0275 | Monoclonal antibody 1F7 |
| Monoclonal antibody 2F5 | Monoclonal antibody 3F12 | Monoclonal antibody 447-52D | Monoclonal antibody 50-61A |
| Monoclonal antibody B4 | Monoclonal antibody HNK20 | Monoclonal antibody NM01 | Mopyridone |
| Moroxydine | Motavizumab | Motexafin gadolinium | Mozenavir |
| MPC 531 | MRK 1 | MS 1060 | MS 1126 |
| MS 8209 | MS 888 | MSC 127 | MSH 143 |
| MTCH 24 | MTP-PE | Murabutide | MV 026048 |
| MX 1313 | Mycophenolate mofetil | Navuridine | NB 001 |
| Neomycin B-arginine conjugate | Neotripterifordin | Nevirapine | Nitric oxide (e.g., ProStrakan) |
| Nitrodeazauridine | NM 01 | NM 49 | NM 55 |
| NNY-RANTES | Nonakine | NP 06 | NP 77A |
| NPC 15437 | NSC 158393 | NSC 20625 | NSC 287474 |
| NSC 4493 | NSC 615985 | NSC 620055 | NSC 624151 |
| NSC 624321 | NSC 627708 | NSC 651016 | NSC 667952 |
| NSC 708199 | NV 01 | Octoxynol 9 | OCX O191 |
| OH 1 | OKU 40 | OKU 41 | Oltipraz |
| Omaciclovir | Opaviraline | OPT TL3 | Oragen |
| ORI 9020 | Oseltamivir | Oxetanocin | Oxothiazolidine carboxylate |
| PA 344/PA 344B | Palinavir | Palivizumab | PAMBAEEG |
| Papuamide A | PBS 119 | PC 1250 | PC 515 |
| PCL 016 | PD 0084430 | PD 144795 | PD 153103 |
| PD 157945 | PD 169277 | PD 171277 | PD 171791 |
| PD 173606 | PD 173638 | PD 177298 | PD 178390 |
| PD 178392 | PD 190497 | Pegaldesleukin | Peldesine |
| PEN 203 | Penciclovir | Pentosan polysulfate | Pentoxifylline |
| Peptide T | Peramivir | PETT 4 | PG 36 |
| Phellodendrine | Phosphatidyllamivudine | Phosphatidylzalcitabine | Phosphatidylzidovudine |
| Phosphazid | Phosphinic cyclocreatine | Pinosylvin | Pirodavir |
| PL 2500 | Pleconaril | Plerixafor | PM 104 |
| PM 19 | PM 523 | PM 92131 | PM 94116 |
| PMEDAP | PMS 601 | PMTG | PMTI |
| PN 355 | PNU 103657 | PNU 142721 | podophyllotoxin |
| Poly ICLC | Polyadenylic polyuridylic acid | Polysaccharide K | PP 29 |
| PPB 2 | PPL 100 | Pradefovir | Pradimicin A |
| Prasterone | PRO 140 | PRO 2000 | PRO 367 |
| PRO 542 | Probucol (Vyrex Corp.) | Propagermanium | Prostratin |
| Pseudohypericin | PSI 5004 | PTPR | PTX 111 |
| Pyriferone | Q 8045 | QM 96521 | QM 96639 |
| QR 435 | Quinobene | Quinoxapeptin A | Quinoxapeptin B |
| QYL 438 | QYL 609 | QYL 685 | QYL 769 |
| R 170591 | R 18893 | R 61837 | R 71762 |
| R 82150 | R 82913 | R 851 | R 87366 |
| R 91767 | R 944 | R 95288 | Raluridine |
| Ramatroban | Ranpirnase | RB 2121 | RBC CD4 |
| RD 30028 | RD 42024 | RD 42138 | RD 42217 |
| RD 42227 | RD 62198 | RD 65071 | RD6 Y664 |
| Regavirumab | Resobene | Respiratory syncytial virus | Retrogen |
| | | immune globulin | Rimantadine |
| REV 123 | RFI 641 | Rilpivirine | Ritonavir |
| RKS 1443 | RO 0334649 | RO 247429 | RO 250236 |
| RO 316840 | RO 53335 | Robustaflavone | Rolipram |
| RP 70034 | RP 71955 | RPI 312 | RPI 856 |
| RPR 103611 | RPR 106868 | RPR 111423 | RS 654 |
| RS 980 | RSV 604 | Rubitecan | Rupintrivir |
| S 1360 | S 2720 | S 9a | SA 1042 |
| SA 8443 | SB 180922 | SB 205700 | SB 206343 |
| SB 73 | SC 49483 | SC 55099 | SCH 350634 |
| SD 894 | S-DABO | SDF 1 | SDZ 282870 |
| SDZ 283053 | SDZ 283471 | SDZ 89104 | SDZ PRI 053 |
| SE 063 | Semapimod | Sevirumab | SF 950 |
| SF 953 | Siamycin 1 | Siamycin 2 | sICAM-1 |
| Sifuvirtide | SIGA 246 | Sizofiran | SJ 3366 |
| SK 034 | SKF 108922 | SKI 1695 | SO 324 |
| Sodium laurilsulfate | Solutein | Sorivudine (e.g., topical) | SP 10 |
| SP 1093V | Sparfosic acid | SPC 3 | SPD 756 |
| SpecifEx-Hep B | SPI 119 | SPL 2992 | SPL 7013 |
| SPV 30 | SR 10204 | SR 10208 | SR 11335 |
| SR 3745A | SR 3773 | SR 3775 | SR 3784 |
| SR 3785 | SR 41476 | SRL 172 | SRR SB3 |
| ST 135647 | Stachyflin | stallimycin | Stampidine |
| Statolon | Stavudine | Stepronin | Suksdorfin |
| Sulfated maltoheptaose | Superoxide dismutase | Suramin (e.g., sodium) | Sy 801 |
| T 1100 | T 118 | T 22 | T 30695 |
| T 611 | T 705 | T4GEN | Tacrine |
| TAK 220 | TAK 652 | TAK 779 | Talviraline |
| TAP 29 | TASP | Teceleukin | Tecogalan (e.g., sodium) |
| TEI 2306 | Telbivudine | Telinavir | Temacrazine |
| Tenidap | Tenofovir | Tenofovir disoproxil fumarate | TGG II 23A |
| TH 9407 | TH 9411 | Thalidomide | Thiophosphonoformic acid |
| Thymoctonan | Thymosin fraction 5 | Thymotrinan | tICAM-1 |
| Tifuvirtide | Tilarginine | Tipranavir | Tiviciclovir |
| Tivirapine | TJ 41 | TL 3024 | TMC 126 |
| TNF-alpha inhibitor | TNK 6123 | TNX 355 | Todoxin |
| Tomeglovir | Transforming growth factor- | TraT | Trecovirsen |
| | alpha | | |
| Tremacamra | Trichosanthin | Triconal | Trimidox |
| Trodusquemine | Tromantadine | Trovirdine | Tuvirumab |
| U 103017 | U 75875 | U 78036 | U 80493 |
| U 81749 | U 88204E | U 96988 | U 9843 |
| UA 926 | Ubenimex | UC 10 | UC 16 |
| UC 38 | UC 42 | UC 68 | UC 70 |
| UC 781 | UC 81 | UC 82 | UIC 94003 |
| Ukrain | UL36ANTI | UMJD 828 | Valaciclovir |
| Valganciclovir | Valomaciclovir | Valtorcitabine | Varicella zoster immune |
| | | | globulin |
| VB 19038 | Vesnarinone | VF 1634 | VGV 1 |
| Vicriviroc | VIR 101 | Viraprexin | Virodene |
| Viscum album extract | VRX 496 | VX 10166 | VX 10217 |
| VX 10493 | VX 11106 | WHI 05 | WHI 07 |
| WIN 49569 | WIN 49611 | WM 5 | WR 151327 |
| XK 216 | XK 234 | XN 482 | XP 951 |
| XQ 9302 | XR 835 | XU 348 | XU 430 |
| Y-ART-3 | YHI 1 | YK FH312 | Z 100 |
| Z 15 | Zalcitabine | Zanamivir | Zidovudine (e.g., phosphate- |
| | | | didanosine dimer) |
| Zidovudine triphosphate mimics | ZX 0610 | ZX 0620 | ZX 0791 |
| ZX 0792 | ZX 0793 | ZX 0851 | ZY II |

TABLE 3
Additional hepatitis C therapies

| | | | |
|---|---|---|---|
| Albuferon | | JTK 003 | R7128 |
| 2′-C-methyl-7-deaza-adenosine | HCV AB 68 | JTK 109 | Resiquimod |
| A-837093 | HCV-SM | KPE 00001113 | Rosiglitazone |
| AG-021541 | HE 2000 | KPE 02003002 | Sargramostim |
| Aldesleukin | Hepatitis C immune globulin | Lactoferrin | |
| ANA 971 | Hepex C | Lamivudine | SCH 6 |
| ANA 975 | Heptazyme | LB 84451 | Schisandra |
| AVI 4065 | Histamine | Licorice root | SCV 07 |
| AVR 118 | Histamine dihydrochloride | ME 3738 | SCY-635 |
| | (e.g., injection, oral) | | |
| Bavituximab | HuMax-HepC | Medusa Interferon | Silipide |
| BILN 303 SE | Hypericin | | Taribavirin |
| BIVN 401 | ICN 17261 | Milk thistle | |
| BLX 833 (e.g., controlled | IDN 6556 | Mitoquinone | Thymalfasin (e.g., Zadaxin) |
| release) | | | |
| | Imiquimod | NIM 811 | Thymus extract |
| CellCept | Interferon | N-nonyl-DNJ | TJ 9 |
| Ceplene | Interferon alfa-2b (e.g., | NOV 205 | Tucaresol |
| | inhalation) | | |
| Ciluprevir (BILN 2061) | Interferon alfacon-1 | NV-08 | Ursodeoxycholic acid |
| Civacir | Interferon alpha (e.g., | P 56 | UT 231B |
| | sustained release, intranasal, | | |
| | Omniferon) | | |
| Colloidal silver | | Peginterferon alfa-2a | Valopicitabine (NM 283) |
| CpG 10101 | Interferon alpha-2b (e.g., | Peginterferon alfa-2b | VGX 410 |
| | controlled release or | | |
| | transdermal) | | |
| DEBIO-025 | Interferon alpha-2b gene | PEGinterferon alfacon-1 | Virostat |
| | therapy | | |
| Edodekin alfa | Interferon alpha-n3 | PEGylated interferon | VP 50406 |
| EHC 18 | Interferon beta-1a | Pegylated thymalfasin | VRT 21493 |
| EMZ 702 | Interferon beta-1b | PF-03491390 | |
| Fas-ligand inhibitor | Interferon gamma-1b | PG 301029 | WF 10 |
| Ginseng | Interferon omega | PSI-6130 | XTL 2125 |
| Glycyrrhizin | Interleukin 10 (e.g., human | R 1518 | XTL 6865 |
| | recombinant) | | |
| GS 9132 | Isatoribine | R 1626 | |
| HCV 086 | ISIS 14803 | R 803 | |
| HCV 371 | ITMN-191 | R-1626 | |

Analogs of any of the compounds listed in Tables 1-3 may be used in any of the compositions, methods, and kits of the invention. Such analogs include, e.g., structural analogs and any agent having the same molecular target(s), e.g., the same enzyme.

Compounds useful in the invention include those described herein in any of their pharmaceutically acceptable forms, including isomers such as diastereomers and enantiomers, salts, solvates, and polymorphs thereof, as well as racemic mixtures. Compounds useful in the invention may also be isotopically labeled compounds. Useful isotopes include hydrogen, carbon, nitrogen, oxygen, phosphorous, fluorine, and chlorine, (e.g., ²H, ³H, ¹³C, ¹⁴C, ¹⁵N, ¹⁸O, ¹⁷O, ³¹P, ³²P, ³⁵S, ¹⁸¹⁸F, and ³⁶Cl). Isotopically-labeled compounds can be prepared by synthesizing a compound using a readily available isotopically-labeled reagent in place of a non-isotopically-labeled reagent.

By an “inhibitor of cholesterol biosynthesis” is meant an agent that inhibits an enzyme of the cholesterol biosynthetic pathway by at least 5%. By a “step of cholesterol biosynthesis” is meant the conversion of a cholesterol precursor to a product by the action of an enzyme during the biosynthesis of cholesterol.

By an “inhibitor of sphingomyelin biosynthesis” is meant an agent that inhibits an enzyme of the sphingomyelin biosynthetic pathway by at least 5%.

By an “inhibitor of cholesterol absorption” is meant an agent that inhibits cellular uptake of cholesterol.

By “patient” is meant any animal (e.g., a mammal such as a human). Any animal can be treated using the methods, compositions, and kits of the invention.

To “treat” is meant to administer one or more agents to measurably slow or stop the replication of a virus in vitro or in vivo, to measurably decrease the load of a virus (e.g., any virus described herein including a hepatitis virus such as hepatitis A, B, C, D, or E) in a cell in vitro or in vivo, or to reduce at least one symptom (e.g., those described herein) associated with having a viral disease in a patient. Desirably, the slowing in replication or the decrease in viral load is at least 20%, 30%, 50%, 70%, 80%, 90%, 95%, or 99%, as determined using a suitable assay (e.g., a replication assay described herein). Typically, a decrease in viral replication is accomplished by reducing the rate of DNA or RNA polymerization, RNA translation, polyprotein processing, or by reducing the activity of .a protein involved in any step of viral replication (e.g., proteins coded by the genome of the virus or host protein important for viral replication).

By “an effective amount” is meant the amount of a compound, alone or in combination with another therapeutic regimen, required to treat a patient with a viral disease (e.g., any virus described herein including a hepatitis virus such as hepatitis A, B, C, D, or E) in a clinically relevant manner. A sufficient amount of an active compound used to practice the present invention for therapeutic treatment of conditions caused by a virus varies depending upon the manner of administration, the age, body weight, and general health of the patient. Ultimately, the prescribers will decide the appropriate amount and dosage regimen. Additionally, an effective amount may be an amount of compound in the combination of the invention that is safe and efficacious in the treatment of a patient having a viral disease over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication.

By “hepatic virus” is meant a virus that can cause hepatitis. Such viruses include hepatitis A, hepatitis B, hepatitis C, hepatitis D, hepatitis E, non-ABCDE hepatitis, and hepatitis G.

By a “low dosage” is meant at least 5% less (e.g., at least 10%, 20%, 50%, 80%, 90%, or even 95%) than the lowest standard recommended dosage of a particular compound formulated for a given route of administration for treatment of any human disease or condition. For example, a low dosage of an agent that inhibits viral replication and that is formulated for administration by intravenous injection will differ, from a low dosage of the same agent formulated for oral administration.

By “hypercholesterolemia” is meant a total cholesterol level of at least 200 mg/dl. High risk groups include those with at least 240 mg/dl. Normal cholesterol levels are below 200 mg/dl. Hypercholesterolemia may also be defined by low density lipoprotein (LDL) levels. Less than 100 mg/dl is considered optimal; 100 to 129 mg/dl is considered near optimal/above optimal; 130 to 159 mg/dl borderline high; 160 to 189 mg/dl high; and 190 mg/dl and above is considered very high.

In the generic descriptions of compounds of this invention, the number of atoms of a particular type in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e., cycloalkyl. Cyclic groups can be monocyclic or polycyclic and preferably have from 3 to 12 ring carbon atoms, inclusive. Exemplary cyclic groups include cyclopropyl, cyclobutyl, cyclopentyl, and cyclohexyl groups.

By “C₁₋₄ alkyl” is meant a branched or unbranched hydrocarbon group having from 1 to 4 carbon atoms. A C₁₋₄ alkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₁₋₄ alkyls include, without limitation, methyl, ethyl, n-propyl, isopropyl, cyclopropyl, cyclopropylmethyl, n-butyl, iso-butyl, sec-butyl, tert-butyl, and cyclobutyl.

By “C₂₋₄ alkenyl” is meant a branched or unbranched hydrocarbon group containing one or more double bonds and having from 2 to 4 carbon atoms. A C₂₋₄ alkenyl may optionally include monocyclic or polycyclic rings, in which each ring desirably has from three to six members. The C₂₋₄ alkenyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, carboxyalkyl, and carboxyl groups. C₂₋₄ alkenyls include, without limitation, vinyl, allyl, 2-cyclopropyl-1-ethenyl, 1-propenyl, 1-butenyl, 2-butenyl, 3-butenyl, 2-methyl-1-propenyl, and 2-methyl-2-propenyl.

By “C₂₋₄ alkynyl” is meant a branched or unbranched hydrocarbon group containing one or more triple bonds and having from 2 to 4 carbon atoms. A C₂₋₄ alkynyl may optionally include monocyclic, bicyclic, or tricyclic rings, in which each ring desirably has five or six members. The C₂₋₄ alkynyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. C₂₋₄ alkynyls include, without limitation, ethynyl, 1-propynyl, 2-propynyl, 1-butynyl, 2-butynyl, and 3-butynyl.

By “C₂₋₆ heterocyclyl” is meant a stable 5- to 7-membered monocyclic or 7- to 14-membered bicyclic heterocyclic ring which is saturated, partially unsaturated, or unsaturated (aromatic), and which consists of 2 to 6 carbon atoms and 1, 2, 3, or 4 heteroatoms independently selected from N, O, and S and including any bicyclic group in which any of the above-defined heterocyclic rings is fused to a benzene ring. The heterocyclyl group may be substituted or unsubstituted. Exemplary substituents include alkoxy, aryloxy, sulfhydryl, alkylthio, arylthio, halide, hydroxy, fluoroalkyl, perfluoralkyl, amino, aminoalkyl, disubstituted amino, quaternary amino, hydroxyalkyl, carboxyalkyl, and carboxyl groups. The nitrogen and sulfur heteroatoms may optionally be oxidized. The heterocyclic ring may be covalently attached via any heteroatom or carbon atom which results in a stable structure, e.g., an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, b-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g., phenyl). The aryl group has from 6 to 12 carbon atoms. Aryl groups may optionally include monocyclic, bicyclic, or tricyclic rings, in which each ring desirably has five or six members. The aryl group may be substituted or unsubstituted. Exemplary substituents include alkyl, hydroxy, alkoxy, aryloxy, sulfhydryl, alkylthio, arylthio, halide, fluoroalkyl, carboxyl, hydroxyalkyl, carboxyalkyl, amino, aminoalkyl, monosubstituted amino, disubstituted amino, and quaternary amino groups.

By “C₇₋₁₄ alkaryl” is meant an alkyl substituted by an aryl group (e.g., benzyl, phenethyl, or 3,4-dichlorophenethyl) having from 7 to 14 carbon atoms.

By “C₃₋₁₀ alkheterocyclyl” is meant an alkyl substituted heterocyclic group having from 3 to 10 carbon atoms in addition to one or more heteroatoms (e.g., 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3, or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted. Exemplary substituents include alkoxy, aryloxy, sulfhydryl, alkylthio, arylthio, halide, hydroxyl, fluoroalkyl, perfluoralkyl, amino, aminoalkyl, disubstituted amino, quaternary amino, hydroxyalkyl, hydroxyalkyl, carboxyalkyl, and carboxyl groups. Examples of C₁₋₇ heteroalkyls include, without limitation, methoxymethyl and ethoxyethyl.

By “halide” or “halogen” is meant bromine, chlorine, iodine, or fluorine.

By “fluoroalkyl” is meant an alkyl group that is substituted with a fluorine atom.

By “perfluoroalkyl” is meant an alkyl group consisting of only carbon and fluorine atoms.

By “carboxyalkyl” is meant a chemical moiety with the formula —(R)—COOH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “quaternary amino” is meant a chemical substituent of the formula —(R)—N(R′)(R″)(R′″)⁺, wherein R, R′, R″, and R′″ are each independently an alkyl, alkenyl, alkynyl, or aryl group. R may be an alkyl group linking the quaternary amino nitrogen atom, as a substituent, to another moiety. The nitrogen atom, N, is covalently attached to four carbon atoms of alkyl, heteroalkyl, heteroaryl, and/or aryl groups, resulting in a positive charge at the nitrogen atom.

Other features and advantages of the invention will be apparent from the following Detailed Description and the claims.

### FIGURES

FIGS. 1(a) and 1(b) represent single agent activity for the chemical probes.

FIGS. 2(a) and 2(b) Are an overview of the HCV replicon and host responses showing the observed combination activity.

FIGS. 3 (a), 3(b) and 3(c) demonstrate the multi-target interactions in the replicon assay.

FIGS. 4(a), 4(b) and 4(c) represent validation experiments.

### DETAILED DESCRIPTION

We have identified compounds that decrease replication of a hepatitis C (HCV) replicon in mammalian cells. Accordingly, the present invention provides compositions, methods, and kits useful in the treatment of viral diseases, which may be caused by a single stranded RNA virus, a flaviviridae virus, or a hepatic virus (e.g., described herein). In certain embodiments, the viral disease is viral hepatitis (e.g., hepatitis A, hepatitis B, hepatitis C, hepatitis D, and hepatitis E). Compositions of the invention can include a combination pair of agents of Table 1. Treatment methods of the invention include administration of a single agent from Table 8 or a pair of agents, e.g., from the pairs of Table 1, optionally along with an additional antiviral therapy (e.g., administration of one or more agents of Table 2 or Table 3) to a patient (e.g., a mammal such as a human). Optionally, functional or structural analogs (e.g., those described herein) of these agents may be employed in the compositions, methods, and kits of the invention. The composition may function by decreasing RNA or DNA polymerization, RNA translation, RNA or DNA transcription, a decrease in posttranslational protein processing (e.g., polyprotein processing in hepatitis C), or a decrease in activity of a protein involved in viral replication (e.g., a protein coded for by the viral genome or a host protein required for viral replication). The compounds or combinations of compounds may also enhance the efficacy of the other therapeutic regimens such, that the dosage, frequency, or duration of the other therapeutic regimen is lowered to achieve the same therapeutic benefit, thereby moderating any unwanted side effects.

In one particular example, the patient being treated is administered a combination of two agents listed in Table 1 within 28 days of each other in amounts that together are sufficient to treat the patient having a viral disease. The two agents can be administered within 14 days of each other, within seven days of each other, within twenty-four hours of each other, or even simultaneously (i.e., concomitantly). If desired, either one of the two agents may be administered in low dosage.

### Viral Diseases

The invention relates to the treatment of viral disease, which can be caused by any virus. Viruses include single stranded RNA viruses, flaviviridae viruses, and hepatic viruses. In particular, the flaviviridae family of viruses includes hepacivirus (e.g., HCV); flaviviruses; pestiviruses, and hepatitis G virus.

Flaviviruses generally are discussed in Chapter 31 of Fields Virology, supra. Exemplary flaviviruses include Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, royal farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, west Nile, Yaounde, yellow fever, and Zika viruses.

Pestiviruses generally are discussed in Chapter 33 of Fields Virology, supra. Specific pestiviruses include, without limitation: bovine viral diarrhea virus, classical swine fever virus (also called hog cholera virus), and border disease virus.

### Hepatitis Viruses

Viruses that can cause viral hepatitis include hepatitis A, hepatitis B, hepatitis C, hepatitis D, and hepatitis E. In addition, non-ABCDE cases of viral hepatitis have also been reported (see, for example, Rochling et al., Hepatology 25:478-483, 1997). Within each type of viral hepatitis, several subgroupings have been identified. Hepatitis C, for example, has at least six distinct genotypes (1, 2, 3, 4, 5, and 6), which have been further categorized into subtypes (e.g., 1a, 1b, 2a, 2b, 2c, 3a, 4a) (Simmonds, J. Gen. Virol. 85:3173-3188, 2004).

In the case of hepatitis C, acute symptoms can include jaundice, abdominal pain, fatigue, loss of appetite, nausea, vomiting, low-grade fever, pale or clay-colored stools, dark urine, generalized itching, ascites, and bleeding varices (dilated veins in the esophagus). Hepatitis C can become a chronic infection, which can lead to liver infection and scarring of the liver, which can, in turn, require the patient to undergo a liver transplant.

Hepatitis C is an RNA virus taken up specifically by hepatic cells. Once inside the cells, the RNA is translated into a polyprotein of about 3,000 amino acids. The protein is then processed into three structural and several non-structural proteins necessary for viral replication. Accordingly, HCV may be treated by reducing the rate any of the steps required for its replication or inhibiting any molecule involved in replication, including but not limited to, entry into a target cell, viral genome replication, translation of viral RNA, protolytic processing, and assembly and release from the target cell (e.g., using the agents described herein).

### Compounds

Certain compounds that may be employed in the methods, compositions, and kits of the present invention are discussed in greater detail below. It will be understood that analogs of any compound of Table 1 or Table 8 can be used instead of the compound of Table 1 or Table 8 in the methods, compositions, and kits of the present invention.

### Cholesterol Biosynthesis Inhibitors

In certain embodiments, a cholesterol biosynthesis inhibitor can be used in the compositions, methods, and kits of the invention. By a “cholesterol biosynthesis inhibitor” is meant a compound that inhibits the activity of an enzyme of the cholesterol biosynthetic pathway by at least 5%, e.g., greater than 10%, 20%, 40%, 60%, 80%, 90%, or 95%. Enyzmes of the cholesterol biosynthetic pathway include HMG-CoA synthase, HMG-CoA reductase, mevalonate kinase, phosphomevalonate kinase, farnesyl transferase, geranylgeranyl transferase, FPP synthase, squalene synthase, squalene monooxygenase, lanosterol synthase, lanosterol 14α-demethylase, Δ14-sterol reductase, C-4 methyl sterol oxidase, 3β-hydroxysteroid dehydrogenase, 3-ketosteroid dehydrogenase, sterol Δ8,Δ7 isomerase, sterol-C5-desaturase, sterol Δ7 reductase, and sterol Δ24 reductase.

### HMG-CoA Reductase Inhibitors

In certain embodiments, an HMG-CoA reductase inhibitor can be used in the compositions, methods, and kits of the invention. By an “HMG-CoA reductase inhibitor” is meant a compound that inhibits the enzymatic activity of 3-hydroxy-3-methylglutaryl-coenzyme A (HMG-CoA) reductase by at least 5%. For example, an HMG-CoA reductase inhibitor may inhibit HMG-CoA reductase by greater than 10%, 20%, 40%, 60%, 80%, 90%, or 95%. HMG-CoA reductase inhibitors include but are not limited to simvastatin (EP0033538B1), lovastatin (GB2046737A), mevastatin (U.S. Pat. No. 3,983,140), pravastatin, monacolin M, monacolin X, fluvastatin (described in PCT publication WO/1984/00213)1, atorvastatin, carvastatin (described in PCT publication WO/1989/08094), cerivastatin, rosuvastatin, fluindostatin, velostatin, acitemate (101197-99-3), acitretin (CAS 55079-83-9), compactin, dihydrocompactin, rivastatin, dalvastatin (CAS 132100-55-1), itavastatin (U.S. Pat. No. 5,011,930), advicor (described in PCT publication WO99/06035), BAY102987, BAY X 2678, BB476, bervastatin (CAS 132017-01-7), BMS-644950 (described in Ahmad et al., J. Med. Chem. 51:2722-2733, 2008), BMS-180431 (described in U.S. Pat. No. 4,824,959), BMY21950, BMY22089, colestolone (described in Green et al., Biochem. J. 135:63-71, 1973), CP-83101 (CAS 120360-17-0), crilvastatin (CAS 120551-59-9), DMP565 (CAS 199480-80-3), glenvastatin (described in EP-00307342), FR901512 (described in Hatori et al., J. Antibiot. 57: 390-393, 2004), L659699 (described in U.S. Pat. No. 4,988,697), L669262 (described in EP-00331250 and EP-00408806), NCX6560 (described in PCT publication WO/2004/105754), NR-300s, P882222, P882284, PD134965, PD135022 (CAS 122548-95-2), rawsonol (CAS 125111-69-5), RBx-10558 (described in PCT publication WO/2004/05250), RP61969, 52467, 52468, SC37111, SC45355, SQ33600 (described in Sliskovic et al., Drug News and Perspectives, 5:517-533), SR12813 (described in U.S. Pat. No. 5,043,330 and PCT Publication WO/2002/95652), SR45023A, tocotrienols (described in Parker et al., J. Biol. Chem. 268:11230-11238, 1993) U20685, U88156, and U-9888 (CAS 190783-55-2), as well as pharmaceutically acceptable salts thereof (e.g., simvastatin sodium, lovastatin sodium, fluvastatin sodium, etc.). In addition, HMG CoA-reductase inhibitors are described in Procopiou, et al. (J. Med. Chem. 36: 3658-3665, 1993) and Chan et al. (J. Med. Chem. 36: 3646-3657, 1993).

Colestolone is an HMG-CoA reductase inhibitor and has the following structure:

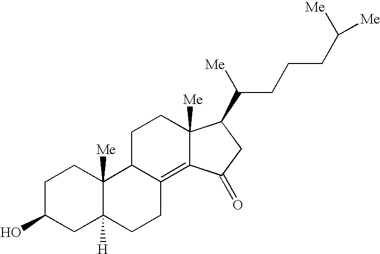

Structural analogs of colestolone include any stereochemical isomer (i.e., enantiomer, diastereomer, or epimer) thereof.

Additional HMG-CoA reductase inhibitors and analogs thereof useful in the methods and compositions of the present invention are described in U.S. Pat. Nos. 3,983,140; 4,231,938; 4,282,155; 4,293,496; 4,294,926; 4,319,039; 4,343,8.14; 4,346,227; 4,351,844; 4,361,515; 4,376,863; 4,444,784; 4,448,784; 4,448,979; 4,450,171; 4,503,072; 4,517,373; 4,661,483; 4,668,699; 4,681,893; 4,719,229; 4,738,982; 4,739,073; 4,766,145; 4,782,084; 4,804,770; 4,824,959; 4,841,074; 4,847,306; 4,857,546; 4,857,547; 4,940,727; 4,946,864; 5,001,148; 5,006,530; 5,075,311; 5,112,857; 5,116,870; 5,120,848; 5,166,364; 5,173,487; 5,177,080; 5,273,995; 5,276,021; 5,369,123; 5,385,932; 5,502,199; 5,763,414; 5,877,208; and 6,541,511; U.S. Pat. Application Publication Nos. 2002/0013334 A1; 2002/0028826 A1; 2002/0061901 A1; and 2002/0094977 A1; and PCT publications WO/2001/96311 and WO/1996/08248.

### Inhibitors of Mevalonate Kinase and Phosphomevalonate Kinase

In certain embodiments, an inhibitor of mevalonate kinase or phosphomevalonate kinase can be used in the compositions, methods, and kits of the invention. By an “inhibitor of mevalonate kinase” is meant a compound that inhibits the enzymatic activity of mevalonate kinase by at least 5%. By an “inhibitor of phosphomevalonate kinase” is meant a compound that inhibits the enzymatic activity of phosphomevalonate kinase by at least 5%. An inhibitor may inhibit mevalonate kinase or phosphomevalonate kinase, e.g., by greater than 10%, 20%, 40%, 60%, 80%, 90%, or 95%. In certain embodiments, an inhibitor may inhibit both mevalonate kinase and phosphomevalonate kinase.

Inhibitors of mevalonate kinase and phosphmevalonate include but are not limited to farnesol and analogs of farnesol. Farnesol has the following structure:

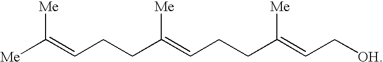

Structural analogs of farnesol may be described by the following formula:

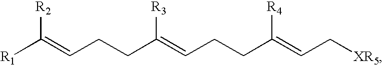

wherein
X is O or NR₆NR₇; and
each R₁-R₇ is selected, independently from H or C₁₋₆ alkyl.

### Inhibitors of Prenyl Transferases

Prenyl transferases include farnesyl transferase and geranylgeranyl transferase. By an “inhibitor of farnesyl transferase” is meant a compound that inhibits the enzymatic activity of farnesyl transferase by at least 5%. By an “inhibitor of geranylgeranyl transferase” is meant a compound that inhibits the enzymatic activity of geranylgeranyl transferase by at least 5%. An inhibitor may inhibit a prenyl transferase, e.g., by greater than 10%, 20%, 40%, 60%, 80%, 90%, or 95%. Compounds that inhibit prenyl transferases include AZD3409, FTI-244, FTI-276, FTI-277, FTI-2148, FTI-2153, FTI-2277, GGTI-286 (described in PCT publication WO/1996/021456), GGTI-287, GGTI-DU40, GGTI-297, GGTI-298, GGTI-2132, GGTI-2133, GGTI-2139, GGTI-2144, GGTI-2145, GGTI-2146, GGTI-2147, GGTI-2151, GGTI-2152, GGTI-2154, GGTI-2157, GGTI-2158, GGTI-2159, GGTI-2160, GGTI-2163, GGTI-2164, GGTI-2165, SCH 663366, and those decribed in U.S. Pat. No. 6,180,619 and U.S. Pat. No. 6,313,109.

GGTI-286 is an inhibitor of geranylgeranyl transferase and has the following structure:

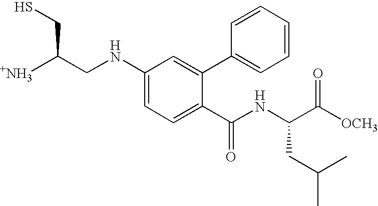

Structural analogs of GGTI-286 are described by formulas A through L in PCT publication WO 96/021456, which is herein incorporated by reference, and are described in Vasudevan et al. (J. Med. Chem. 42:1333-1340, 1999). Analogs of GGTI-286 may inhibit farnesyl transferase or geranyl geranyl transferase.

### Inhibitors of Farnesyl Diphosphate Synthase

In certain embodiments, an inhibitor of farnesyl diphosphate synthase can be used in the compositions, methods, and kits of the invention. By an “inhibitor of farnesyl diphosphate synthase” is meant a compound that inhibits the enzymatic activity of farnesyl diphosphate synthase by at least 5%. For example, a farnesyl diphosphate synthase inhibitor may inhibit farnesyl diphosphate synthase by greater than 10%, 20%, 40%, 60%, 80%, 90%, or 95%. Inhibitors of farnesyl diphosphate synthase include but are not limited to alendronate, pamidronate, risedronate, and ibandronate.

Alendronate has the following structure

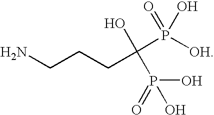

Structural analogs of alendronate include any salts thereof. Other structural analogs are found in Belgium Patent No. 903510, U.S. Pat. No. 4,705,651, and in EP0537008, each of which is herein incorporated by reference. Other structural analogs may be described using the following formula:

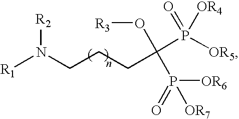

wherein
each R₁, R₂, and R₃ is selected, independently, from H, C₁₋₆ alkyl, C(O)R₈, CO₂R₈, or CONR₈R₉, where each R₈ and R₉ is, independently, H or C₁₋₆ alkyl;
n is 0, 1, 2, 3, 4, 5, or 6;
each R₄, R₅, R₆, and R₇ is selected, independently, from H, C₁₋₆ alkyl, or optionally substituted phenyl, wherein a substituted phenyl has 1, 2, 3, 4, or 5 substituents selected, independently, from halogen, NO₂, C₁₋₆ alkyl, or C₁₋₆ alkoxy.

### Inhibitors of Squalene Synthase

In certain embodiments, an inhibitor of squalene synthase can be used in the compositions, methods, and kits of the invention. By an “inhibitor of squalene synthase” is meant a compound that inhibits the enzymatic activity of squalene synthase by at least 5%. For example, a squalene synthase inhibitor may inhibit squalene synthase by greater than 10%, 20%, 40%, 60%, 80%, 90%, or 95%. Inhibitors of squalene synthase include but are not limited to squalestatin and TAK-475.

### Inhibitors of Squalene Monooxygenase

In certain embodiments, an inhibitor of squalene monooxygenase can be used in the compositions, methods, and kits of the invention. By an “inhibitor of squalene monooxygenase” is meant a compound that inhibits the enzymatic activity of squalene monooxygenase by at least 5%. By an “inhibitor of squalene monooxygenase” is meant a compound that inhibits the enzymatic activity of squalene monooxygenase by at least 5%. For example, a squalene monooxygenase inhibitor may inhibit squalene monooxygenase by greater than 10%, 20%, 40%, 60%, 80%, 90%, or 95%. Squalene monooxygenase inhibitors include but are not limited to clomiphene (U.S. Pat. No. 2,914,563), terbinafine, naftifine, tolnaftate, Tu-2208, FR-194738 (CAS 204067-52-7), NB-598, SDZ-87-469 (CAS 87906-31-8), FW-1045, Ro-44-2104 (CAS 140620-63-9), SDZ-880-540 (CAS 121242-84-0), CAS 168414-53-7, and SDZ-SBA-586 (CAS 164411-48-7).

Clomiphene has the following structure:

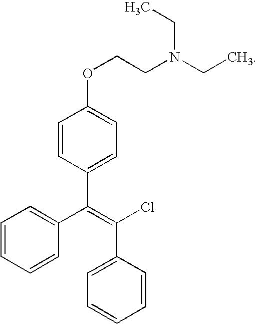

Structural analogs of clomiphene include olefinic isomers. Structural analogs of clomiphene are also described by the following formula:

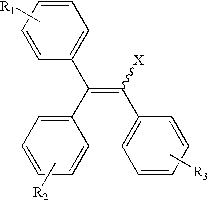

wherein
X is any halogen (e.g., F, Cl, Br, or I)
R₁, R₂, and R₃ may be located at any position of the phenyl group and are selected, independently, from H, halogen, C₁₋₆ alkyl, C₁₋₆ alkoxy, —OCₙH₂ₙA, and
at least one of R₁, R₂, and R₃ is —OCₙH₂ₙA, wherein
   n is 2, 4, 5, or 6;
   A=NR₄R₅, wherein each R₄ and R₅ is, independently, an optionally substituted C₁₋₆ alkyl, or R₄ and R₅ combined to form an optionally substituted cyclic structure.
      Desirably, when R₁, R₂, or R₃ is —OCₙH₂ₙA, the substituents is located para to the olefin substituents. Examples of C₁₋₆ alkyls include, but are not limited to: methyl, ethyl, propyl, isopropyl, n-butyl, iso-butyl, sec-butyl, tert-butyl, n-pentyl, isoamyl, and n-hexyl. Examples of C₁₋₆ alkoxy groups include, but are not limited to: methoxy, ethoxy, propoxy, isopropoxy, n-butyloxy, iso- butyloxy, sec- butyloxy, tert-butyloxy, n-pentoxy, O-isoamyl, and O-hexyl. Examples of rings formed by the combination R₄ and R₅ include, but are not limited to pyrrolidine and piperidine.

Other analogs are described in U.S. Pat. No. 2,914,563; the general formula of U.S. Pat. No. 5,410,080; and in U.S. Pat. No. 5,189,212, each of which is incorporated herein by reference.

### Inhibitors of Lanosterol Synthase

In certain embodiments, an inhibitor of lanosterol synthase can be used in the compositions, methods, and kits of the invention. By an “inhibitor of lanosterol synthase” is meant a compound that inhibits the enzymatic activity of 2,3 oxidosqualene cyclase by at least about 10%. Inhibitors of lanosterol synthase include but are not limited to Ro-48-8071 (U.S. Pat. No. 5,106,878), BIBB-515 (U.S. Pat. No. 5,466,687), BIBB-1464, BIBX-245, BIBX-79 (CAS 175033-26-8), ZD-9720, CAS 153715-95-8, CAS 188526-43-4 (described in publication WO98/35959), substituted isoquinaline derivatives 3 (described in Barth et al., J. Med. Chem. 39: 2303-2312, 1996), pyridinium ion-based inhibitors (eg., N-(4E,8E)-5,9,13,-trimethyl-4,8,12-tetradecatrien-1-ylpyridinium and N-(4E,8E)-5,9,13-trimethyl-4,8,12-tetradecatrien-1-ylpicolinium, described in Goldman et al., Antimicrob. Agents Chemother. 40: 1044-104, 1996), and heteroaromate inhibitors (described in U.S. Pat. No. 7,173,043).

Ro 48-8071 has the following structure:

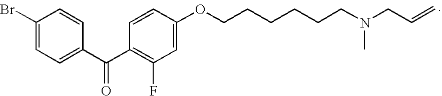

Structural analogs of Ro 48-8071 are described in U.S. Pat. Nos. 5,495,048 and 5,637,771, each of which is incorporated herein by reference. Analogs of Ro 48-8071 can also described by the following formula:

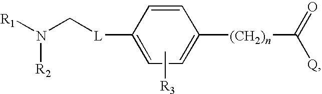

wherein
each R₁ and R₂ is, independently, C₁₋₇ alkyl or C₂₋₇ alkenyl;
L is —(C₁₋₁₁ alkyl)-, —(C₁₋₁₁ alkenyl)-, -(phenyl)-, —(C₁₋₁₁ alkyl-O)—, or —(C₁₋₁₁ alkenyl-O)—;
n is 0 or 1;
Q is C₁₋₇ alkyl, C₂₋₁₀ alkenyl, or —(Ar)—, wherein Ar is an optionally substituted phenyl having 1, 2, or 3 substituents selected from H, CF₃, CN, NO₂, C₁₋₄ alkyl, or halogen (i.e., F, Cl, Br, or I); and
R₃ is H, C₁₋₄ alkyl, or halogen (i.e., F, Cl, Br, or I).
   Exemplary analogs of Ro 48-8071 include, but are not limited to:
4-[[6-(allylmethylamino)hexyl]oxy]-3-chlorobenzophenone;
4-[[6-(allylmethylamino)hexyl]oxy]-3,4′-dibromobenzo phenone;
4-[[4-(allylmethylamino)-2-butenyl]oxy]-3,4′-dibromobenzophenone;
3-chloro-4′-iodo-4-[[6-(allylmethylamino)hexyl]oxy]benzophenone;
4′-bromo-3-chloro-4-[[6-(allylmethylamino)hexyl]oxy]benzophenone;
2,4-[[(4-dimethylamino)-2-butenyl]oxy]-3,4′-dibromobenzophenone;
4-[[4-(dimethylamino)-2-butenyl]oxy]-3-chlorobenzophenone;
4′-bromo-3-chloro-4-[[6-(dimethylamino)hexyl]oxy]benzophenone;
3,4-dichlorophenyl 4′-[(dimethylamino)methyl]-4-biphenylyl ketone;
4′-[(allylmethylamino)methyl]-4-biphenylyl 3,4-dichlorophenyl ketone;
(RS)-4′-(dimethylaminomethyl)-4-biphenyl 2,6-dimethyl-5-heptenyl ketone;
p-bromophenyl 2-chloro-4′-[(dimethylamino)methyl]-4-biphenylyl ketone;
4′-[(dimethylamino)methyl]-4-biphenylylpropyl ketone;
[4-[6-(allyl-methyl-amino)-hexyloxy]-phenyl]-(4-bromo-phenyl)-methanone;
[4-[4-(allyl-methyl-amino)-butoxy]-phenyl]-(4-bromo-phenyl)-methanone;
[4-[6-(allyl-methyl-amino)-hexyloxy]-3-fluoro-phenyl]-(4-bromo-phenyl)-methanone;
[4-[6-(allyl-methyl-amino)-hexyloxy]-2-fluoro-phenyl]-(4-bromo-phenyl)-methanone;
(E)-[4-[4-(allyl-methyl-amino)-but-2-enyloxy]-phenyl]-(4-trifluoromethyl-phenyl)-methanone;
(E)-4-[4-[4-(allyl-methyl-amino)-but-2-enyloxy]-3-fluoro-phenyl]-(4-bromo-phenyl)-methanone;
(E)-1-[4-[4-(allyl-methyl-amino)-but-2-enyloxy]-phenyl]-5-methyl-hexan-1-one;
(E)-[4-[4-(allyl-methyl-amino)-but-2-enyloxy]-phenyl]-(4-iodophenyl)-methanone;
(E)-1-[4-(allyl-methyl-amino)-but-2-enyloxy]-3-fluoro-phenyl]-5-methyl-hexan-1-one;
(E)-[4-[4-(allyl-methyl-amino)-but-2-enyloxy]-3-fluoro-benzoyl]-benzonitrile;
(E)-4-[4-[4-(allyl-methyl-amino)-but-2-enyloxy]-benzoyl]-benzonitrile;
(E)-[4-[4-(allyl-methyl-amino)-but-2-enyloxy]-3-fluoro-phenyl]-(2,6-difluoro-phenyl)-methanone; (E)-1-[4-[4-(allyl-methyl-amino)-but-2-enyloxy]-phenyl]-5-methyl-hex-4-en-1-one;
(E)-[4-[4-(allyl-methyl-amino)-but-2-enyloxy]-2-fluoro-phenyl]-(4-bromo-phenyl)-methanone;
(E)-[4-[4-(allyl-methyl-amino)-but-2-enyloxyl-phenyl]-(4-fluoro-phenyl)-methanone;
(E)-1-[4-[4-(allyl-methyl-amino)-but-2-enyloxy]-3-fluoro-phenyl]-6-methyl-hept-5-en-2-one;
(E)-2-[4-[4-(allyl-methyl-amino)-but-2-enyloxy]-3-fluoro-phenyl]-1-(4-bromo-phenyl)-ethanone;
(E)-2-[4-[4-(allyl-methyl-amino)-but-2-enyloxy)-phenyl]-1-(4-bromo-phenyl)-ethanone;
(E)-(4-bromo-phenyl)-[4-[4-(ethyl-methyl-amino)-but-2-enyloxy]-phenyl]-methanone;
4′-[(allylmethylamino)methyl]-2-chloro-4-biphenylyl p-bromophenyl ketone; and
4′-[(allylmethylamino)methyl]-4-biphenyl 4-methyl-3-pentenyl ketone.

BIBB-515 is also known as 1-(4-chlorobenzoyl)-4-(4-(2-oxazolin 2-yl) benzylidene))piperidine and has the following structure:

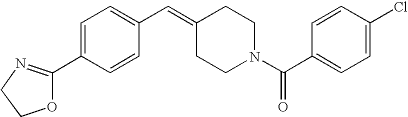

Structural analogs of BIBB-515 are described in U.S. Pat. No. 5,466,687 (see, for example, Formula I and Ia and the compounds of Examples 1-3), which is hereby incorporated by reference.

Structural analogs of BIBB-515 are also described by the' following formula:

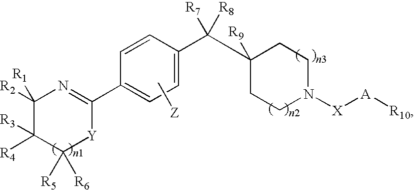

wherein
each n1, n2, and n3 is, independently, 0, 1, or 2;
each R₁-R₆ is, independently, H, optionally substituted C₁₋₆ alkyl, or R₁ and R₅, or R₁ and R₄, or R₂ and R₄, or R₂ and R₅, or R₃ and R₅, or R₃ and R₆, or R₄ and R₆, or R₄ and R₅, combine to form a carbon-carbon double bond;
R₇ is H, or optionally substituted C₁₋₆ alkyl;
R₈ and R₉ are each H or R₈ and R₉ combine to form a carbon-carbon double bond;
Z is H, halogen, or optionally substituted C₁₋₆ alkyl;
X is —C(O)— or —S(O)₂—;
A is a single bond, —(C₁₋₆ alkyl)-, —(C₂₋₆ alkenyl)-, or —(C₂₋₆ alkynyl)-; and
R₁₀ is selected from:
   H,
   C₃₋₆-cycloalkyl group;
   optionally phenyl group, wherein a substituted phenyl group has 1, 2, 3, 4, or 5 substituents selected from halogen, C₁₋₆ alkyl, —CF₃, C₁₋₆ alkoxy, cyano, nitro, C₁₋₆ alkylsulphonyl, or phenyl;
   optionally substituted naphthyl or tetrahydronaphthyl, wherein a substituted naphthyl or tetrahydronaphthyl has 1, 2, 3, or 4 substituents selected from halogens;
   optionally substituted pyridyl or a thienyl, wherein a substituted pyridyl or a thienyl has 1, 2, or 3 substituents selected from halogen or C₁₋₆ alkyl.

Analogs of BIBB-515 include, for example:
1-(4-chlorobenzoyl)-4-[4-(2-oxazolin-2-yl)benzylidene]piperidine
1-(4-chlorobenzoyl)-4-[4-(4,5-dihydro-6H-oxazin-2-yl)benzylidene]piperidine
4-[4-(2-oxazolin-2-yl)benzylidene]-1-(4-trifluoromethylbenzoyl)piperidine
1-(4-chloro-3-methylbenzoyl)-4-[4-(2-oxazolin-2-yl)benzylidene]piperidine
1-(4-fluorobenzoyl)-4-[4-(2-oxazolin-2-yl)benzylidene]piperidine
1-(5-chloro-2-thienoyl)-4-[4-(2-oxazolin-2-yl)benzylidene]piperidine
1-cyclohexanecarbonyl-4-[4-(2-oxazolin-2-yl)benzylidene]piperidine
4-[4-(2-oxazolin-2-yl)benzyl]-1-(4-trifluoromethylbenzoyl)piperidine
1-(4-chlorobenzoyl)-4-[4-(2-oxazolin-2-yl)benzyl]piperidine
1-(4-chlorobenzoyl)-3-[4-(2-oxazolin-2-yl)benzylidene]pyrrolidine
1-(4-chlorobenzoyl)-4-[2-fluoro-4-(2-oxazolin-2-yl)benzylidene]piperidine
1-(4-chlorobenzoyl)-4-[3-methyl-4-(2-oxazolin-2-yl)benzylidene]piperidine
1-(4-chlorobenzenesulphonyl)-4-[4-(2-oxazolin-2-yl)benzylidene]piperidine
1-(4-chlorobenzenesulphonyl)-4-[4-(2-imidazolin-2-yl)benzylidene]piperidine
1-(4-chlorobenzoyl)-4-[4-(2-thiazolin-2-yl)benzyl]piperidine
1-(4-chlorobenzoyl)-4-[4-(S-5-methyl-2-oxazolin-2-yl)benzylidene]piperidine
1-(4-chlorobenzoyl)-4-[4-(R-4-methyl-2-oxazolin-2-yl)benzylidene]piperidine
1-(4-chlorobenzoyl)-4-[4-(5-phenyl-2-oxazolin-2-yl)benzylidene]piperidine
1-(4-chlorobenzoyl)-4-[4-(5-diethylaminomethyl-2-oxazolin-2-yl)benzylidene]piperidine
1-(4-chlorobenzoyl)-4-[4-(4-hydroxymethyl-2-oxazolin-2-yl)benzylidene]piperidine
4-[4-(S-4-benzyl-2-oxazolin-2-yl)benzylidene]-1-(4-chlorobenzoyl)piperidine
1-(4-chlorobenzoyl)-4-[4-(2-oxazolin-2-yl)-.alpha.-methylbenzylidene]piperidine
1-(5-chloro-2-thienoyl)-4-[4-(2-oxazolin-2-yl)benzylidene]piperidine
1-(4-cyanobenzoyl)-4-[4-(2-oxazolin-2-yl)benzylidene]piperidine
4-[4-(2-oxazolin-2-yl)benzylidene]-1-(pentafluorobenzoyl)piperidine
1-benzoyl-4-[4-(2-oxazolin-2-yl)benzylidene]piperidine
1-(4-tert.butylbenzoyl)-4-[4-(2-oxazolin-2-yl)benzylidene]piperidine
1-(4-methoxybenzoyl)-4-[4-(2-oxazolin-2-yl)benzylidene]piperidine
1-(4-bromobenzoyl)-4-[4-(2-oxazolin-2-yl)benzylidene]piperidine
1-(4-nitrobenzoyl)-4-[4-(2-oxazolin-2-yl)benzylidene]piperidine
1-(4-chlorophenylacetyl)-4-[4-(2-oxazolin-2-yl)benzylidene]piperidine
1-(1-naphthylcarbonyl)-4-[4-(2-oxazolin-2-yl)benzylidene]piperidine U18666A.
   Inhibitors of Lanosterol 14α-demethylase

In certain embodiments, inhibitors of lanosterol 14α-demethylase can be used in the compositions, methods, and kits of the invention. By an “inhibitor of lanosterol 14α-demethylase” is meant a compound that inhibits the enzymatic activity of lanosterol 14α-demethylase by at least 5%. Inhibitors of lanosterol 14α-demethylase include but are not limited to terconazole, bifonazole, butoconazole, fluconazole, itraconazole, miconazole, voriconazole, SKF104976, posaconazole (described in PCT publication WO95/17407), DIO-902 (described in PCT publication WO2006/72881), fenticonazole (described in U.S. Pat. No. 4,221,803), cephalosporins (described in WO98/58932), omoconazole (CAS 74512-12-2), Ro-09-1470 (CAS 135357-96-9). Other compounds that may inhibit lanosterol 14α-demethylase include amorolfine and fenpropimorph.

Terconazole is described in U.S. Pat. No. 4,144,346 and has the following structure:

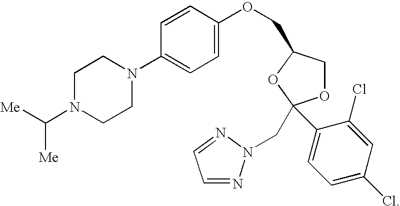

Structural analogs of terconazole include any stereochemical isomers thereof. Other structural analogs are described in U.S. Pat. Nos. 3,575,999, 3,936,470, 4,223,036, 4,358,449 (see, for example, Examples I-LXXII), in Belgian Patent No. 93.5,579, and in the PCT Publication No. WO00/76316, each of which is hereby incorporated by reference.

Structural analogs of terconazole can also be described by the following formula:

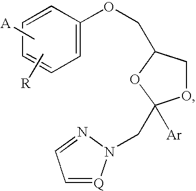

wherein
Q is —CH— or —N—;
Ar is optionally substituted phenyl, wherein a substituted phenyl has 1, 2, or 3 substituents that are, independently, halogen, C₁₋₆ alkyl, or C₁₋₆ alkoxy;
A is —NCS, NR₂R₃, —NHC(X)—(Y)ₘ—R₄, or

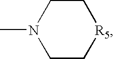

wherein
each R₂ and R₃ is, independently, H or C₁₋₆ alkyl;
X is O or W;
Y is O or NH;
m is 0 or 1;
R₄ is H, optionally substituted C₁₋₆ alkyl, or optionally substituted phenyl, wherein a substituted C₁₋₆ alkyl, or substituted phenyl has 1 or 2 substituents that are each, independently, halogen, C₁₋₆ alkyl, or C₁₋₆ alkoxy;
R₅ is a bond, —CH₂—, —O—, —S—, or —NR₆—, where R₆ is H or optionally substituted C₁₋₆ alkyl; and
R is H or NO₂.

Exemplary, non-limiting structural analogs of terconazole are:
4-[2-(3-chlorophenyl)-2-(1H-imidazol-1-ylmethyl)-1,3-dioxolan-4-ylmethoxy]-N-ethylbenzenamine;
4-[2-(4-bromophenyl)-2-(1H-imidazol-1-ylmethyl)-1,3-dioxolan-4-ylmethoxy]-N-ethylbenzenamine;
N-ethyl-4-[2-(1H-imidazol-1-ylmethyl)-2-(3-methylphenyl)-1,3-dioxolan-4-ylmethoxy]benzenamine;
N-ethyl-4-[2-(1H-imidazol-1-ylmethyl)-2-(4-methoxyphenyl)-1,3-dioxolan-4-yl methoxy]benzenamine;
4-[2-(2,4-dichlorophenyl)-2-(1H-1,2,4-triazol-1-ylmethyl)-1,3-dioxolan-4-yl methoxy]-N-ethylbenzenamine;
N-{4-[2-(3-chlorophenyl)-2-(1H-imidazol-1-ylmethyl)-1,3-dioxolan-4-ylmethoxy]phenyl}acetamide.
N-{4-[2-(4-bromophenyl)-2-(1H-imidazol-1-ylmethyl)-1,3-dioxolan-4-ylmethoxy]phenyl}benzamide
ethyl{4-[2-(1H-imidazol-1-ylmethyl)-2-(3-methylphenyl)-1,3-dioxolan-4-ylmethoxy]phenyl}carbamate;
N-{4-[2-(1H-imidazol-1-ylmethyl)-2-(4-methoxyphenyl)-1,3-dioxolan-4-ylmethoxy]phenyl}-4-fluorobenzamide;
N-{4-[2-(2,4-dichlorophenyl)-2-(1H-1,2,4-triazol-1-ylmethyl)-1,3 dioxolan-4-ylmethoxy]phenyl}acetamide;
1-{2-(3-chlorophenyl)-4-[4-(1-pyrrolidinyl)phenoxymethyl]-1,3-dioxolan-2-yl methyl}-1H-imidazole;
1-{2-(4-bromophenyl)-4-[4-(1-piperidinyl)phenoxymethyl]-1,3-dioxolan-2-ylmethyl}-1H-imidazole;
1-{2-(3-methylphenyl)-4-[4-(1-pyrrolidinyl)phenoxymethyl]-1,3-dioxolan-2-yl methyl}-1H-imidazole;
1-{2-(4-methoxyphenyl)-4-[4-(1-piperidinyl)phenoxymethyl]-1,3-dioxolan-2-yl methyl}-1H-imidazole;
1-{2-(2,4-dichlorophenyl)-4-[4-(1-pyrrolidinyl)phenoxymethyl]-1,3-dioxolan-2-ylmethyl}-1H-1,2,4-triazole;
1-{2-(2,4-dichlorophenyl)-4-[4-(1-piperidinyl)phenoxymethyl]-1,3-dioxolan-2-ylmethyl}-1H-1,2,4-triazole;
4-{4-[2-(3-chlorophenyl)-2-(1H-imidazol-1-ylmethyl)-1,3-dioxolan-4-ylmethoxy]phenyl}morpholine;
4-{4-[2-(1H-imidazol-1-ylmethyl)-2-(3-methylphenyl)-1,3-dioxolan-4-ylmethoxy]phenyl}morpholine;
4-{4-[2-(1H-imidazol-1-ylmethyl)-2-(4-methoxyphenyl)-1,3-dioxolan-4-ylmethoxy]phenyl}morpholine; and
4-{4-[2-(2,4-dichlorophenyl)-2-(1H-1,2,4-triazol-1-ylmethyl)-1,3-dioxolan-4-ylmethoxy]phenyl}morpholine.
   Inhibitors of Δ14-sterol Reductase and Sterol Δ7,Δ8-sterol Isomerase

In certain embodiments, inhibitors of Δ14-sterol reductase and sterol Δ7Δ8-isomerase can be used in the compositions, methods, and kits of the invention. By an “inhibitor of Δ14-sterol reductase” is meant a compound that inhibits the enzymatic activity of Δ14-sterol reductase by at least 5%. By an “inhibitor of sterol Δ7Δ8-isomerase” is meant a compound that inhibits the enzymatic activity of Δ7Δ8-isomerase by at least 5%. For example, an inhibitor used in the invention may inhibit the activity of Δ14-sterol reductase or sterol Δ7Δ8-isomerase by greater than 10%,20%, 40%, 60%, 80%, 90%, or 95%. An inhibitor of Δ14-sterol reductase may also inhibit sterol Δ7Δ8-isomerase. An inhibitor of Δ14-sterol reductase may also inhibit sterol Δ7Δ8-isomerase. Inhibitors of Δ14-sterol reductase include but are not limited to amorolfine (described in EP0024334) and fenpropimorph. Inhibitors of sterol Δ7Δ8-isomerase include but are not limited to amorolfine, fenpropimorph, SR31747, and trans-1,4-diaminocyclohexanes (described in PCT Publication WO02/51793).

Amorolfine is an antifungal agent that is typically administered topically. The structure of amorolfine is:

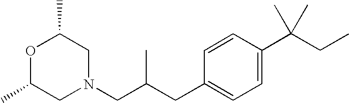

Analogs of amorolfine are described, for example, in U.S. Pat. No. 4,202,894 and have the general structure:

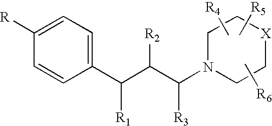

wherein R is alkyl of 4 to 12 carbon atoms, cycloalkyl of 3 to 7 carbon atoms, mono(lower alkyl)-substituted cycloalkyl of 4 to 7 carbon atoms, cycloalkylalkyl of 4 to 12 carbon atoms, phenyl or aryl-(lower alkyl) of 7 to 12 carbon atoms; R₁, R₂, and R₃, independently, are hydrogen or alkyl of 1 to 8 carbon atoms; R₄, R₅, and R₆, independently, are hydrogen or alkyl of 1 to 8 carbon atoms, and two of R₄, R₅, and R₆ can each be bonded to the same carbon atom or together can form a fused alicyclic or aromatic 6-membered ring; provided that when R is tert.-butyl, at least one of R₁ and R₃ is alkyl of 2 to 8 carbon atoms or R₂ is hydrogen or alkyl of 2 to 8 carbon atoms or at least one of R₄, R₅, and R₆ is alkyl of 5 to 8 carbon atoms; X is methylene or an oxygen atom; z is zero or 1 and the dotted bonds can be hydrogenated, and acid addition salts of those compounds of formula I which are basic, where the term “lower alkyl” denotes a straight-chain or branched-chain hydrocarbon group of 1 to 4 carbon atoms, such as, methyl, ethyl, propyl, isopropyl, butyl, isobutyl and tert.-butyl. Alkyl groups of 4 to 12 carbon atoms are straight-chain or branched-chain hydrocarbon groups, for example, butyl, isobutyl, tert.-butyl, neopentyl, 1,1-dimethylpropyl, 1,1-dimethylpentyl, 1,1-diethylpropyl, 1,1-dimethylbutyl, 1-isopropyl-3-methyl-but-1-yl, 1-ethyl-1-methylbutyl, dodecyl, and the like. Cycloalkylalkyls include, in particular, those groups in which the alkyl moiety is branched. The term “aryl-(lower alkyl)” includes not only groups which are mono- or di(lower alkyl)-substituted in the aryl ring but also groups which are mono- or di(lower alkyl)-substituted in the lower alkyl moiety. Exemplary of aryl(lower alkyl) groups are benzyl, phenylethyl, (lower alkyl)-benzyl, for example, methylbenzyl and dimethylbenzyl, naphthylmethyl, 2-phenyl-propan-2-yl, 1-phenyl-1-ethyl, or the like.

Amorolfine is a member of the morpholines, which include ((2-azido-4-benzyl)phenoxy)-N-ethylmorpholine, (+)-(S)-5,5-dimethylmorpholinyl-2-acetic acid, (morpholinyl-2-methoxy)-8-tetrahydro-1,2,3,4-quinoline, 1,1′-hexamethylenebis(3-cyclohexyl-3-((cyclohexylimino)(4-morpholinyl)methyl)urea), 1,4-bis(3′-morpholinopropyl-1′-yl-1′)benzene, 1,4-thiomorpholine-3,5-dicarboxylic acid, 1,4-thiomorpholine-3-carboxylic acid, 1-(morpholinomethyl)-4-phthalimidopiperidine-2,6-dione, 1-deoxy-1-morpholino-psicose, 1-deoxy-1-morpholinofructose, 1-phenyl-2,3-dimethyl-4-naphthalanmorpholinomethylpyrazolin-5-one, 1-phenyl-2-palmitoylamino-3-morpholino-1-propanol, 2,6-bis(carboxymethyl)-4,4-dimethylmorpholinium, 2,6-dimethylmorpholine, 2,6-dioxo-N-(carboxymethyl)morpholine, 2-(((3-(morpholinylmethyl)-2H-chromen-8-yl)oxy)methyl)morpholine, 2-(3-trifluoromethyl)phenyltetrahydro-1,4-oxazine, 2-(4-morpholino)ethyl-1-phenylcyclohexane-1-carboxylate, 2-(4-morpholino-6-propyl-1,3,5-triazin-2-yl)aminoethanol, 2-(4-morpholinyl)-4H-1-benzopyran-4-one, 2-(4-morpholinyl)-8-phenyl-4H-1-benzopyran-4-one, 2-(4-nitrophenyl)-4-isopropylmorpholine, 2-(morpholin-4-yl)benzo(h)chromen-4-one, 2-(N-methylmorpholinium)ethyl acetate, 2-(N-morpholino)ethanesulfonic acid, 2-benzylmorpholine, 2-hydroxy-4,4-dimethyl-2-(4-tolyl)morpholinium, 2-methyl-3-(2-methyl-2,3-diphenyl-4-morpholinyl)-1-phenyl-1-propanone, 2-morpholinomethyl-2′,3′,4′-trimethoxyacrylophenone, 2-n-pentyloxy-2-phenyl-4-methylmorpholine, 2-phenyl-5,5-dimethyltetrahydro-1,4-oxazine, 2-thiomorpholinoethylacrylamide, 3,5,5-trimethyl-2-morpholinon-3-yl radical dimer, 3-((benzyloxy)methyl)morpholine, 3-(beta-morpholinoethoxy)-1H-indazole, 3-cyano-2-morpholino-5-(pyrid-4-yl)pyridine, 3-thiomorpholinopropylacrylamide, 4,4′-dithiodimorpholine, 4,4-methylenedimorpholine, 4-(2-morpholinoethoxy)benzophenone, 4-(3,7,11,15-tetramethyl-6,10,14-hexadecatrienoyl)morpholine, 4-amino-5-chloro-2-ethoxy-N-((2-morpholinyl)methyl)benzamide, 4-amino-N-((4-benzyl-2-morpholinyl)-methyl)-5-chloro-2-ethoxybenzamide, 4-amino-N-((4-benzyl-2-morpholinyl)methyl)-5-chloro-2-methoxybenzamide, 4-benzylphenoxy-N-ethylmorpholine, 4-cyclododecyl-2,6-dimethylmorpholine acetate, 4-methoxyphenyl-(5-methyl-6-(2-(4-morpholinyl)ethyl)-6H-thieno(2,3-b)pyrrol-4-yl)phenylmethanone, 4-methylmorpholine, 4-methylmorpholine N-oxide, 4-morpholinedithiocarbamate, 4-morpholinocarbonitrile, 5-pentyl-N-nitrosomorpholine, A 74273, AH 19437, aprepitant, AWD 140076, befol, BIBW 22, bis(3,5-dimethyl-5-hydroxymethyl-2-oxomorpholin-3-yl), BW 175, cetethyl morpholinium, CGP 53437, CI1033, ciclosidomine, CNK 6001, CNK 6004, CP 80794, CP 84364, CS 722, delmopinol, detensitral, dextromoramide, di-beta-(morpholinoethyl)selenide, dimethomorph, dimethyl morpholinophosphoramidate, dimorpholamine, ES 6864, ES 8891, fenpropimorph, filenadol, FK 906, fominoben, FR 76830, Go 8288, GYM 11679, indeloxazine, L 689502, L 742694, L 760735, landiolol, lateritin, M&B 16573, MDL 101146, MF 268, mofarotene, molsidomine, morfolep, moricizine, morlincain, moroxybrate, moroxydine, morpholine, morpholineoethylamino-3-benzocyclohepta(5,6-c)pyridazine, morpholinoamidine, morpholinophosphordichloridite, morpholinopropane sulfonic acid, morpholinosulfonic acid, morpholinylethoxy-3-methyl-4-(2′-naphthyl)-6-pyridazine, mosapride, N,N′-dicyclohexyl-4-morpholinecarboxamidine, N-((4-benzyl-2-morpholinyl)methyl)-5-chloro-4-(dimethylamino)-2-methoxybenzamide, N-(3,N′-morpholinopropyl)-2-(3-nitropyrrolo-(2,3-b)pyridine-1-yl)ethanoic acid amide, N-(3-nitro-4-quinoline)morpholino-4-carboxamidine, N-dodecylmorpholine, N-ethylmorpholine, N-hexylmorpholine-2′,5′-oligoadenylate, N-nitromorpholine, N-oxydiethylene-2-benzothiazole sulfenamide, O—(N-morpholinocarbonyl)-3-phenyllactic acid, oxaflozane, oxymorphindole, P 1487, P 34081, PD 132002, phendimetrazine, phenmetrazine, phenyl 2-(2-N-morpholinoethoxy)phenyl ether, pholcodine, phosphorodiamidate morpholino oligomer, pinaverium, pramoxine, proctofoam-HC, promolate, RE 102, reboxetine, Ro 12-5637, Ro 12-8095, RS 1893, RV 538, S 12024, S 14001, S-anisylformamidino-4-(N-methylisothioamide)morpholine, S-phenethylformamidino-4-(N-ethylisothioamide)morpholine, SC 46944, Seda-Miroton, silatiemonium iodide, SIN 1C, SR 121463A, stymulen, sufoxazine, teomorfolin, theniloxazine, thiamorpholine, tiemonium iodide, tiemonium methylsulfate, tridemorph, trifenmorph, trimetozine, trimorfamid, trithiozine, TVX 2656, U⁻37883A, U 84569, U 86983, UP 614-04, viloxazine, Win 55212-2, and YM 21095.

Fenpropimorph has the following structure:

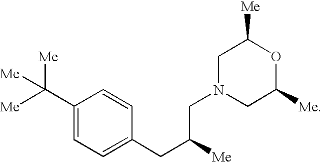

Structural analogs of are described in DE2656747, herein incorporated by reference. Structural analogs also include any stereochemical isomers of fenpropimorph or of any analogs thereof.

Structural analogs of fenpropimorph can also be described using the following formula:

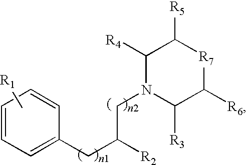

wherein
R₁ is C₁₋₆ alkyl and can be at any position of the phenyl ring
each n1 and n2 is, independently, 0, 1, 2, 3, 4, 5, 6, or 7;
R₂ is C₁₋₆ alkyl;
each R₃, R₄, R₅, and R₆ is, independently, H or C₁₋₆ alkyl; and
R₇ is —CR₈R₉—, —O—, or —NR₈—, wherein each R₈and R₉ is, independently, H or C₁₋₆ alkyl.

Alternative morpholine compounds that may be used in the invention are described in PCT publication WO02/51793 and in U.S. Pat. No. 4,301,284.

Inhibitors of 3β-hydroxysteroid Dehydrogenase

In certain embodiments, inhibitors of 3β-hydroxysteroid dehydrogenase can be used in the compositions, methods, and kits of the invention. By an “inhibitor of 3β-hydroxysteroid dehydrogenase” is meant a compound that inhibits the enzymatic activity of 3β-hydroxysteroid dehydrogenase by at least 5%. For example, an inhibitor used in the invention may inhibit the activity of 3β-hydroxysteroid dehydrogenase reductase by greater than 10%, 20%, 40%, 60%, 80%, 90%, or 95%. Inhibitors of 3β-hydroxysteroid dehydrogenase include but are not limited to trilostane (CAS 13647-35-3) and analogs of trilostane.

### Inhibitors of Sterol Δ7 Reductase

In certain embodiments, inhibitors of sterol Δ7 reductase can be used in the compositions, methods, and kits of the invention. By an “inhibitor of sterol Δ7 reductase” is meant a compound that inhibits the enzymatic activity of sterol Δ7 reductase by at least 5%.

For example, an inhibitor used in the invention may inhibit the activity of sterol Δ7 reductase by greater than 10%, 20%, 40%, 60%, 80%, 90%, or 95%. Inhibitors of sterol Δ7 reductase include but are not limited to AY-9944 (described in Dvornik et al., J. Am. Chem. Soc. 85: 3309, 1963) and BM15766.

AY-9944 has the following structure:

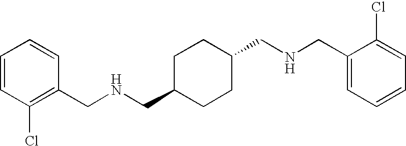

Structural analogs of AY-9944 include the cis-stereoisomer. Other structural analogs of AY-9944 may be described by the following formula:

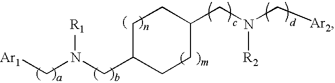

wherein
each n and m is, independently, 0, 1, 2, or 3;
each a, b, c, and d, is, independently, 0, 1, 2, 3, 4, 5, 6, 7, or 8;
each R₁ and R₂, is, independently, H or C₁₋₆ alkyl; and
each Ar₁ and Ar₂ is, independently, optionally substituted phenyl, wherein a substituted phenyl has 1, 2, 3, 4, or 5 substituents selected, independently, from halogen, C₁₋₆ alkyl, or C₁₋₆ alkoxy.

Sterol isomerase inhibitors described in publication WO/2002/051793 may also be useful in certain aspects of the invention as inhibitors of sterol Δ7 reductase.

### Inhibitors of Sterol Δ24 Reductase

In certain embodiments, inhibitors of sterol Δ24 reductase can be used in the compositions, methods, and kits of the invention. By an “inhibitor of sterol Δ24 reductase” is meant a compound that inhibits the enzymatic activity of sterol Δ24 reductase by at least 5%. For example, an inhibitor used in the invention may inhibit the activity of sterol Δ24 reductase by greater than 10%, 20%, 40%, 60%, 80%, 90%, or 95%. Inhibitors of sterol Δ24 reductase include but are not limited to triparanol (CAS 78-41-1, described in U.S. Pat. No. 2,914,561), brassicasterol, and U-18666A (CAS 3039-71-2).

### Inhibitors of Cholesterol Absorption

In certain embodiments, inhibitors of cholesterol absorption, such as azetidine compounds or inhibitors of acyl-CoA cholesterol acyltransferase, can be used in the compositions, methods, and kits of the invention. Inhibitors of cholesterol absorption measurably reduce cellular uptake of cholesterol. Inhibitors of cholesterol absorption include but are not limited to ezetimibe, FM-VP4 (described in PCT publication WO01/00653), β-sitosterol, campesterol (CAS 474-62-4), stigmasterol, stigmastanol, Sandoz 58-035, CP-113818, TMP-153, HL-004, CL277082, SMP-500, VULM-1457, and YIC-C8-434.

The structure of ezetimibe is:

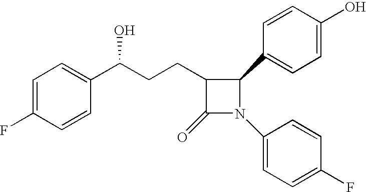

Analogs of ezetimibe may also be used in certain embodiments of the invention. Ezetimibe analogs are described, for example, in U.S. Pat. No. 5,767,115 and are described by the formula:

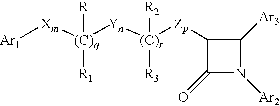

where Ar₁ and Ar₂ are independently selected from the group consisting of aryl and R₄-substituted aryl; Ar₃ is aryl or R₅-substituted aryl; X, Y and Z are independently selected from the group consisting of —CH₂—, —CH(lower alkyl)- and —C(dilower alkyl)-; R and R₂ are independently selected from the group consisting of —OR₆, —O(CO)R₆, —O(CO)OR₉ and —O(CO)NR₆R₇; R₁ and R₃ are independently selected from the group consisting of hydrogen, lower alkyl and aryl; q is 0 or 1; r is 0 or 1; m, n and p are independently 0, 1, 2, 3 or 4; provided that at least one of q and r is 1, and the sum of m, n, p, q and r is 1, 2, 3, 4, 5 or 6; and provided that when p is 0 and r is 1, the sum of m, q and n is 1, 2, 3, 4 or5; R₄ is 1-5 substituents independently selected from the group consisting of lower alkyl, —OR₆, —O(CO)R₆, —O(CO)OR₉, —O(CH₂)₁₋₅OR₆, —O(CO)NR₆R₇, —NR₆R₇, —NR₆(CO)R₇, —NR₆(CO)OR₉, —NR₆(CO)NR₇R₈, —NR₆SO₂R₉, —COOR₆, —CONR₆R₇, —COR₆, —SO₂NR₆R₇, S(O)₀₋₂R₉, —O(CH₂)₁₋₁₀—COOR₆, —O(C₂)₁₋₁₀CONR₆R₇, -(lower alkylene)COOR₆, —CH═CH—COOR₆, —CF₃, —CN, —NO₂ and halogen; R₅ is 1-5 substituents independently selected from the group consisting of —OR₆, —O(CO)R₆, —O(CO)OR₉, —O(CH₂)₁₋₅OR₆, —O(CO)NR₆R₇, —NR₆R₇, —NR₆(CO)R₇, —NR₆(CO)OR₉, —NR₆(CO)NR₇R₈, —NR₆SO₂R₉, —COOR₆, —CONR₆R₇, —COR₆, —SO₂NR₆R₇, S(O)₀₋₂R₉, —O(CH₂)₁₋₁₀—COOR₆, —O(CH₂)₁₋₁₀CONR₆R₇, -(lower alkylene)COOR₆ and —CH═CH—COOR₆; R₆, R₇ and R₈ are independently selected from the group consisting of hydrogen, lower alkyl, aryl and aryl-substituted lower alkyl; and R₉ is lower alkyl, aryl or aryl-substituted lower alkyl. R₄ is preferably 1-3 independently selected substituents, and R₅ is preferably 1-3 independently selected substituents. Preferred are compounds of formula I wherein Ar₁ is phenyl or R₄-substituted phenyl, especially (4-R₄)-substituted phenyl. Ar₂ is preferably phenyl or R₄-substituted phenyl, especially (4-R₄)-substituted phenyl. Ar₃ is preferably R₅-substituted phenyl, especially (4-R₅)-substituted phenyl. When Ar₁ is (4-R₄)-substituted phenyl, R₄ is preferably a halogen. When Ar₂ and Ar₃ are R₄- and R₅-substituted phenyl, respectively, R₄ is preferably halogen or —OR₆ and R₅ is preferably —OR₆, wherein R₆ is lower alkyl or hydrogen. Especially preferred are compounds wherein each of Ar₁ and Ar₂ is 4-fluorophenyl and Ar₃ is 4-hydroxyphenyl or 4-methoxyphenyl.

Other azetidines include 1,4-bis(4-methoxyphenyl)-3-(3-phenylpropyl)-2-azetidinone, 1-(N-(3-ammoniopropyl)-N-(n-propyl)amino)diazen-1-ium-1,2-diolate, 1-methyl-2-(3-pyridyl)azetidine, 2-oxo-3-phenyl-1,3-oxazetidine, 2-tetradecylglycidyl-coenzyme A, 3-(2-oxopropylidene)azetidin-2-one, 3-aminonocardicinic acid, 3-phenyl-2-methylazetidine-3-ol, 4-((4-carboxyphenyl)oxy)-3,3-diethyl-1-(((phenylmethyl)amino)carbonyl)-2-azetidinone, 4-(3-amino-2-oxoazetidinonyl-1)methylbenzoic acid, 4-(3-amino-2-oxoazetidinonyl-1)methylcyclohexanecarboxylic acid, AHR 11748, azetidine, azetidine platinum(II), azetidinecarboxylic acid, azetidyl-2-carboxylic acid, azetirelin, BDF 9148, BMS-262084, E 4695, fluzinamide, L 652117, L 684248, N-(2-chloromethylphenyl)-3,3-difluoroazetidin-2-one, SCH 60663, SF 2185, tabtoxinine beta-lactam, tazadolene succinate, and ximelagatran.

### Inihibitors of Sphingomyelin Biosynthesis

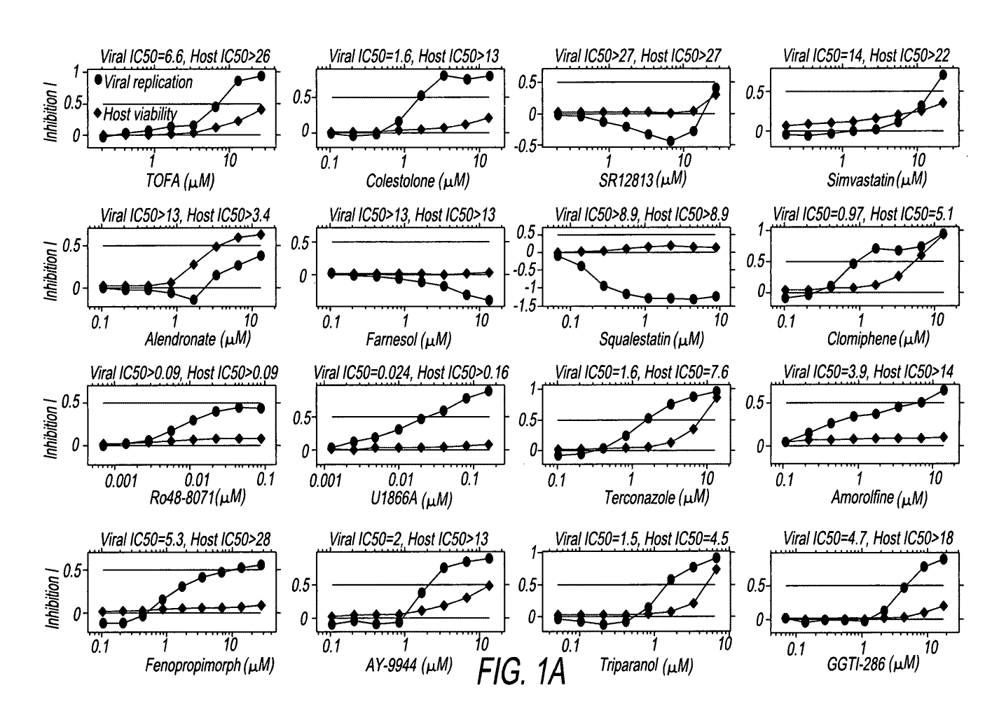

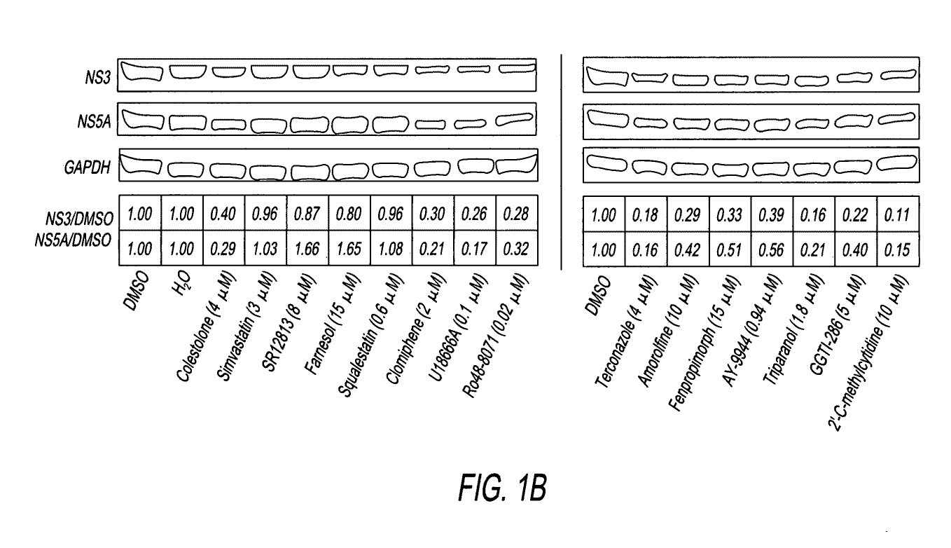

In certain embodiments, inhibitors of sphingolipid biosynthesis can be used in the compositions, methods, and kits of the invention. The sphingomyelin biosynthetic pathway is illustrated schematically in FIG. 1. By an “inhibitor of sphingolipid biosynthesis” is meant a compound that inhibits an enzyme of the sphingomyelin biosynthetic pathway by at least 5%. For example, an inhibitor of sphingolipid biosynthesis used in the invention may inhibit the activity of, eg., acetyl-CoA carboxylase 2 or serine palmitoyl transferase, by greater than 10%, 20%, 40%, 60%, 80%, 90%, or 95%. Inhibitors of Acetyl-CoA carboxylase 2 include but are not limited to TOFA, the FM-TP5000 series of small molecule inhibitors (Forbes Medi-Tech, Inc.), CP-640186 (described in Treadway et al, Diabetes 53:(Abs 679-P), N-{3-[2-(4-alkoxyphenoxy)thiazol-5-yl]-1-methylprop-2-ynyl}carboxy derivatives (described in Gu et al., J. Med. Chem. 49:3770-3773, 2006 and Gu et al., J. Med. Chem. 50:1078-1082, 2007), andrimid (CAS 108868-95-7), and moiramide B (CAS 155233-31-1). Inhibitors of serine palmitoyl transferase include but are not limited to myriocin (U.S. Pat. No. 3,928,572), sphingofungin B and sphingofungin C (described in EP0301744 and Middlesworth et al., J. Antibiot., 45:861-867,1992), L-cycloserine (CAS 339-72-0) and β-chloro-L-alanine (described in Hanada et al., Biochem. Pharmacol., 59:1211-1216, 2000).

In certain embodiments, 5-(tetradecyloxy)-2-furancarboxylic acid (TOFA) or an analog thereof can be used in the compositions, methods, and kits of the invention. TOFA is an inhibitor of acetyl-CoA carboxylase and is described in U.S. Pat. No. 4,110,351. The structure of TOFA is:

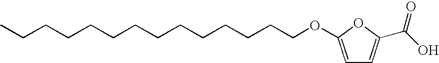

Analogs of TOFA are described, for example, in U.S. Pat. No. 4,382,143 and have the general structure:

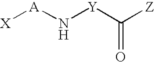

wherein X is selected from the group consisting of hydrogen, C₃-C₈ cycloalkyl, and substituted or unsubstituted aryl; A is a divalent radical selected from the group consisting of branched or unbranched C₆-C₁₉ alkylene, alkenylene, and alkynylene; Y is a 5- or 6-membered heteroaryl ring containing one or more nitrogen, sulfur, or oxygen atoms and optionally unsubstituted or substituted with one fluoro; and Z is selected from the group consisting of hydrogen, hydroxy, loweralkoxy, loweralkoxyloweralkoxy, diloweralkylaminoloweralkoxy, (mono- or polyhydroxy)loweralkoxy, (mono- or polycarboxy)loweralkoxy, (mono- or polycarboxy)hydroxyloweralkoxy, allyloxy, epoxypropoxy, substituted or unsubstituted-(phenoxy, benzyloxy, or 3-pyridyloxy), pyridylmethoxy, tetrahydropyranyloxy, (mono- or polyhydroxy)alkylamino, allylamino, propargylamino, 2-sulfoethylamino, (mono- or polycarboxyl)loweralkylamino, loweralkanoylamino, (substituted or unsubstituted)aroylamino, loweralkanesulfonylamino, (substituted or unsubstituted)arenesulfonylamino, loweralkanylhydrazino, hydroxylamino, polymethyleneimino, and (4-carboxy- or 4-carboethoxy)thiazolidino; and the pharmaceutically acceptable acid-addition and cationic salts thereof.

In certain embodiments, myriocin or an analog thereof can be used in the compositions, methods, and kits of the invention. Myriocin is also known as ISP-1 or thermozymodicin and has the following structure:

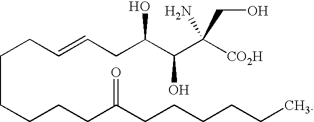

Structural analogs of myriocin include any stereochemical isomer (i.e., olefinic isomers or enantiomer, diastereomers, or epimers) thereof. FTY720 is an exemplary, non-limiting structural analog of myriocin. Other structural analogs of myriocin are described in EP1795206, U.S. Pat. No. 7,189,748, and PCT Publication No. WO2006/042278, each of which is herein incorporated by reference.

Structural analogs of myriocin are also be described by the following formula:

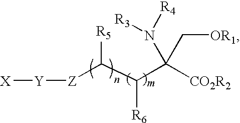

wherein
each R₁, R₂, R₃, and R₄ is selected, independently, from H or optionally substituted C₁₋₆ alkyl, wherein a substituted C₁₋₆ alkyl has 1, 2, 3, 4, or 5 substituents selected from C₁₋₃ alkyl, C₁₋₆ alkoxy, or halogen;
each n and m is 0, 1, 2, 3, 4, or 5;
each R₅ and R₆ is selected, independently, from H, C₁₋₆ alkyl, OR₇, or NR₇R₈, wherein each R₇ and R₈ is selected, independently, from H or C₁₋₆ alkyl;
Z is a single bond, -(optionally substituted C₁₋₆ alkyl)-, -(optionally substituted E- or Z—C₂₋₆ alkenyl) -, or -(optionally substituted C₂₋₆ alkynyl)-, wherein a -(substituted C₁₋₆ alkyl)-, -(substituted E- or Z—C₂₋₆ alkenyl) -, or -(substituted C₂₋₆ alkynyl)-, has 1, 2, 3, 4, 5, or 6 substituents selected, independently, from C₁₋₆ alkyl, OR₇, or NR₇R₈, wherein each R₇ and R₈ is selected, independently, from H or C₁₋₆ alkyl;
Y is a single bond, —C(O)— or —S(O₂)—;
X is an optionally substituted C₁₋₆ alkyl, wherein a substituted C₁₋₆ alkyl has 1, 2, 3, 4, or 5 substituents selected, independently, from C₁₋₆ alkyl, OR₇, or NR₇R₈, wherein each R₇ and R₈ is selected, independently, from H or C₁₋₆ alkyl.

### Other Agents

Other agents that may modulate cholesterol and lipid metabolism can be used in certain embodiments of the invention, including without limitation clofibrate, cerulenin, 7-dehydrocholesterol, lycopene, β-sitosterol, cholesteryl acetate, cholesteryl arachidonate, cholesteryl hexanoate, cholesteryl linoleate, cholesteryl oleate, cholesteryl palmitate cholesteryl stearate, diethylumbelliferyl phosphate, apolipoprotein A-I, apolipoprotein C-I, apolipoprotein C-II, apolipoprotein C-III, apolipoprotein E2, apolipoprotein E3, apolipoprotein E4, fenofibrate, gemfibrozil, nicotinic acid, probucol, and (z)-guggulsterone.

### Sertraline and Analogs Thereof

In certain embodiments, sertraline or an analog thereof can be used in the compositions, methods, and kits of the invention. Sertraline has the structure:

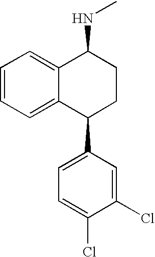

Structural analogs of sertraline are those having the formula:

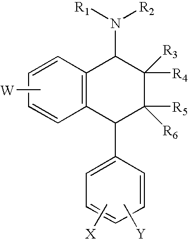

where R₁ and R₂ are independently selected from the group consisting of H, optionally substituted C₁₋₆ alkyl (e.g., CH₃, (CH₂)ₓOH, cyclopropyl, (CH₂)ₓCOOH, or CH₂CHOH(CH₂)ₓ, (CH₂)ₓN(CfH₃)₂, where x is 1, 2, 3, 4, or 5), and optionally substituted C₁₋₇ heteroalkyl (e.g., CH₂CH₂N(CH₃)₂) or R₁ and R₂ together form a C₃₋₈ cycloalkyl optionally heterocyclic, optionally substituted (e.g., forming a morpholine ring), R₃, R₄, R₅, and R₆ are independently H, Cl, F, Br, OH, or optionally substituted C₁₋₆ alkyl; X and Y are each selected from the group consisting of H, F, Cl, Br, CF₃, C₁₋₆ alkoxy (e.g., OPh and OCH₃), and cyano; and W is selected from the group consisting of H, F, Cl, Br, CF₃, C₁₋₃ alkoxy, COOH, CH₂CH₂OH, NHCOH, NHCOCH₃, CH₂S(O)ₙCH₃, CH₂NH₂, CONH₂, CH₂OH, NHCOPh, CH₂NHS(O)ₙCH₃, NHS(O)-Ph, N(CH₃)₂, S(O)ₙNH₂, NHCOBu, NHS(O)ₙCH₃, NHCOcyclopentyl, CN, NHS(O)ₙcyclopropyl, NH₂, NO₂, I, SO₂N(CH₃)₂, SO₂NHMe, SO₂NHCH₂CH₂OH, CO₂Me, NHSO₂Bu, CONHCH₃, CH₂NHCOCH₃, CONHPh,

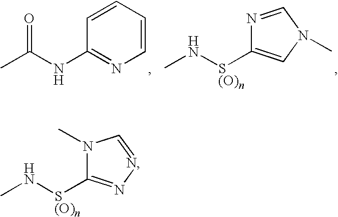

CONHcylopropyl, C(S)NH₂, NHC(S)CH₃, CONHCH₂COOCH₃, CONHCH₂COOH, CONHCH₂cyclopropyl, CONHcyclobutyl, NHCOcyclopropyl, NH(CH₃)COCH₃, and CH₂S(O)ₙR₁₁, where n is 0, 1, or 2 and R₁₁ is phenyl, C₂₋₆ heterocyclyl, optionally substituted C₁₋₈ alkyl (e.g., C₄₋₈ unsubstituted alkyl such as Bu or C₃₋₈ substituted alkyl). In certain embodiments, R₁ is CH₃ and R₂ is CH₃, CH₂CH₂OH, cyclopropyl, CH₂COOH, CH₂CH₂NH₂, CH₂CH(OH)R₈, or CH₂CH(R₈)NR₉R₁₀, where n is 0, 1, or 2 and R₈, R₉, and R₁₀ are independently H or C₁₋₆ alkyl. In certain embodiments, X is H and Y is p-OPh, p-OCF₃, o-OCH₃ m-OCH₃, or p-OCH₃. In certain embodiments of the above structure, the sertraline analog has the formula:

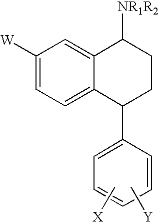

Other sertraline analogs have the formula:

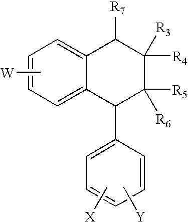

where R₃, R₄, R₅, R₆, W, X, and Y are as defined above, and R₇ is independently H, NH(CH₂)ₘCH₃, O(CH₂)ₘCH₃, OH, O(CH₂)ₘCH₃, ═O, C₁₋₆ alkyl (e.g., isopropyl), or C₁₋₆ alkyoxy, where m is 0, 1, 2, 3, 4, 5, or 6. In certain embodiments, R₃, R₄, R₅, and R₆ are H; X and Y are each Cl at the 3 and 4 positions of the benzyl ring. Exemplary analogs include:

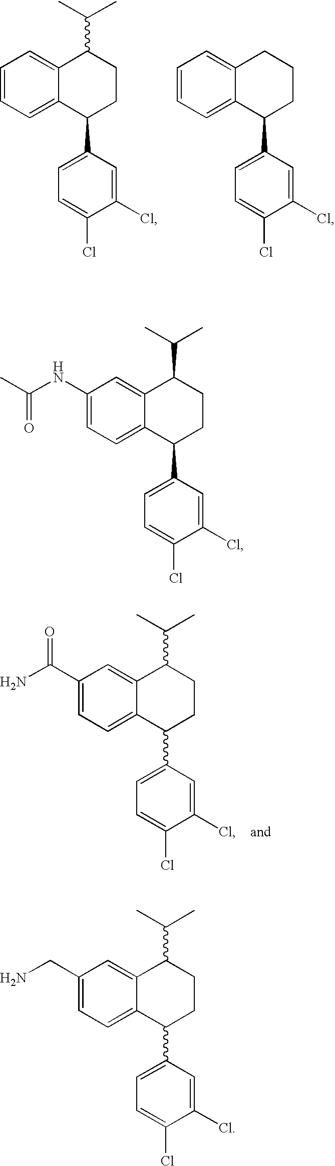

Other sertraline analogs have the formula:

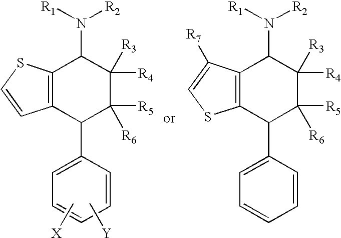

where R₁, R₂, R₃, R₄, R₅, R₆, X and Y are as defined above, and R₇ is H or C₁₋₆ optionally substituted alkyl.

Other sertraline analogs are described by the formula:

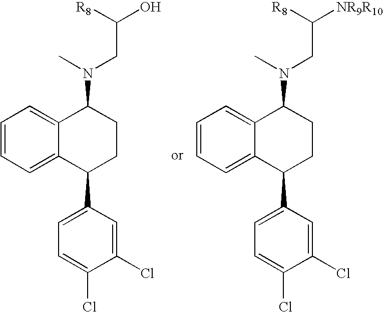

wherein R₈, R₉, and R₁₀ are independently H, optionally substituted C₁₋₆ alkyl (e.g., CH₃, (CH₂)ₓOH, cyclopropyl, (CH₂)ₓCOOH, or CH₂CHOH(CH₂)ₓ, (CH₂)ₓN(CfH₃)₂, x where is 1, 2, 3, 4, or 5), and optionally substituted C₁₋₇ heteroalkyl (e.g., CH₂CH₂N(CH₃)₂)

In certain embodiments, sertraline analogs are in the cis-isomeric configuration. The term “cis-isomeric” refers to the relative orientation of the NR₁R₂ and phenyl moieties on the cyclohexene ring (i.e., they are both oriented on the same side of the ring). Because both the 1- and 4- carbons are asymmetrically substituted, each cis-compound has two optically active enantiomeric forms denoted (with reference to the 1-carbon) as the cis-(1R) and cis-(1S) enantiomers. Sertraline analogs are also described in U.S. Pat. No. 4,536,518. Other related compounds include (S,S)-N-desmethylsertraline, rac-cis-N-desmethylsertraline, (1S,4S)-desmethyl sertraline, 1-des (methylamine)-1-oxo-2-(R,S)-hydroxy sertraline, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH₂, sertraline sulfonamide ethanol, sertraline nitrile, sertraline-CME, dimethyl sertraline reverse sulfonamide, sertraline reverse sulfonamide (CH₂ linker), sertraline B-ring ortho methoxy, sertraline A-ring methyl ester, sertraline A-ring ethanol, sertraline N,N-dimethylsulfonamide, sertraline A-ring carboxylic acid, sertraline B-ring para-phenoxy, sertraline B-ring para-trifluoromethane, N,N-dimethyl sertraline B-Ring para-trifluoromethane, sertraline A-ring methyl sulfoxide (CH₂ linker), sertraline A-ring carboxamide, sertraline A-ring reverse carboxamide, Sertraline A-ring methanamine, sertraline A-ring sulfonylmethane (CH₂ linker), sertraline (reverse) methanesulfonamide, sertraline A-ring thiophene, reduced sulfur sertraline A-ring methyl sulfoxide (CH₂ linker), and heterocyclic substituted stertraline (reverse) methanesulfonamide. Structures of these analogs are shown in Table 4 below.

TABLE 4
Sertraline analogs.

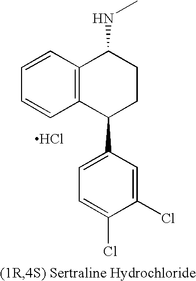

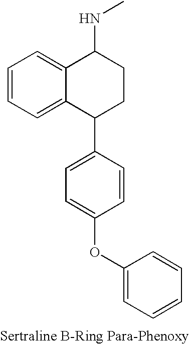

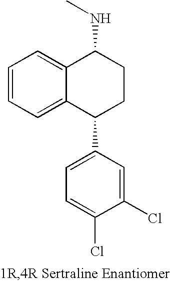

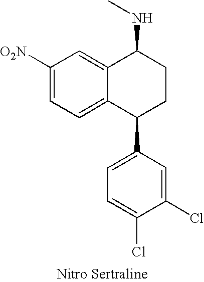

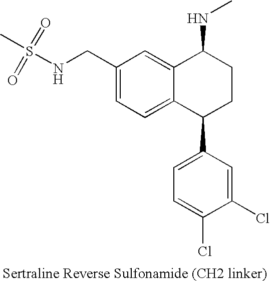

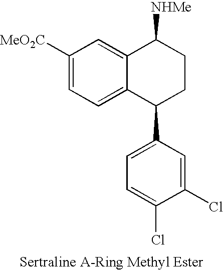

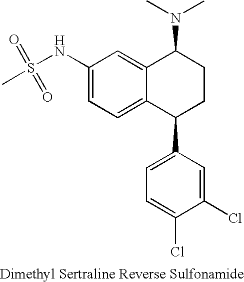

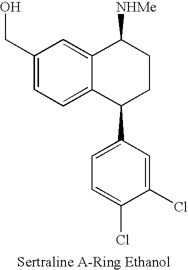

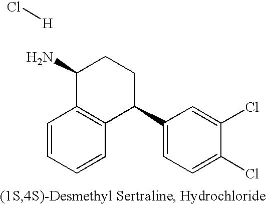

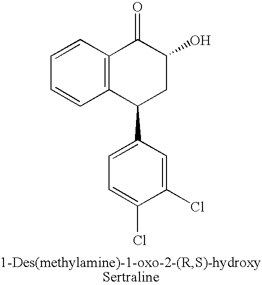

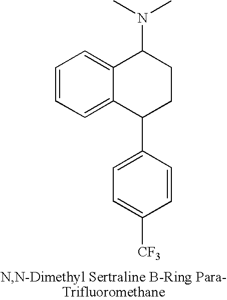

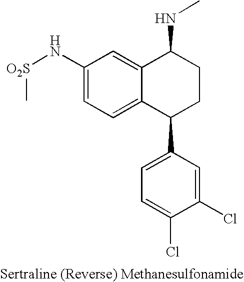

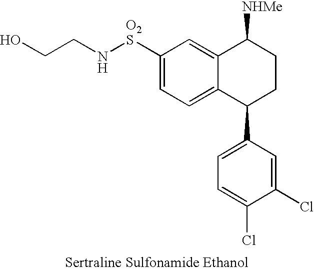

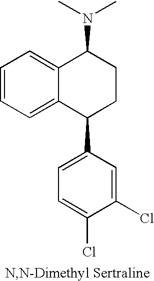

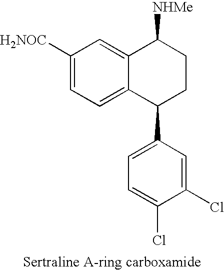

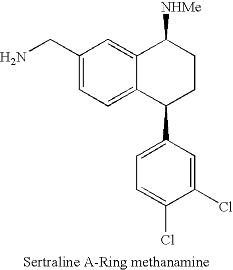

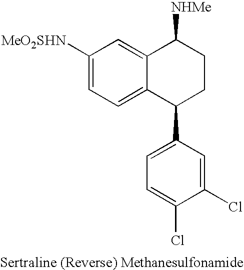

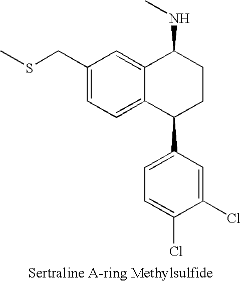

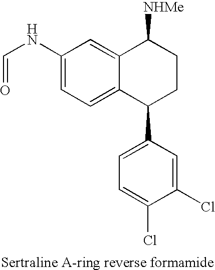

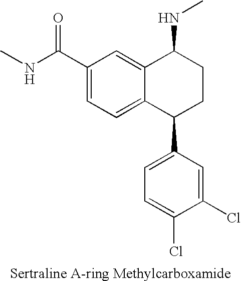

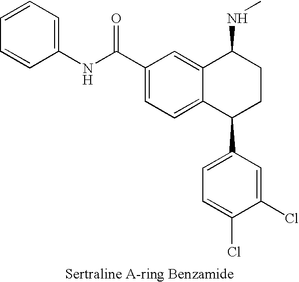

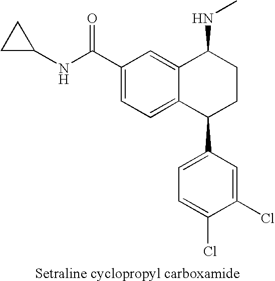

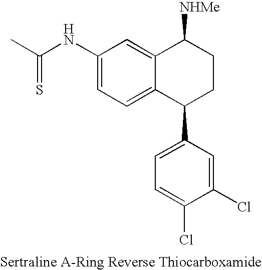

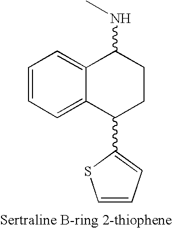

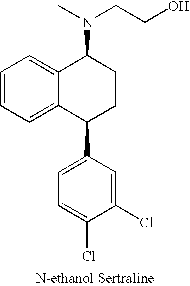

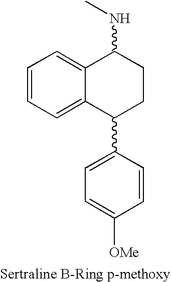

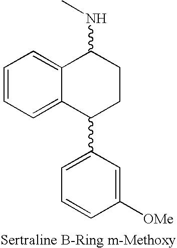

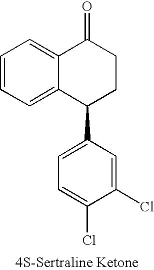

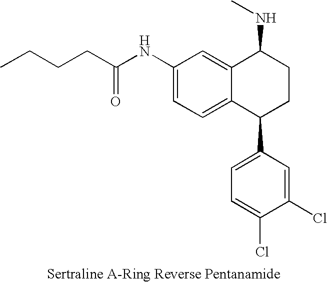

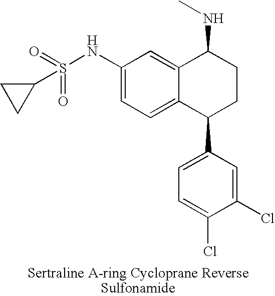

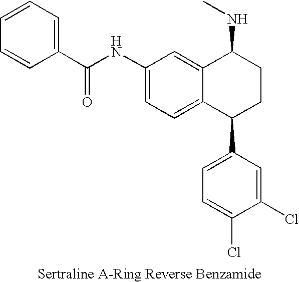

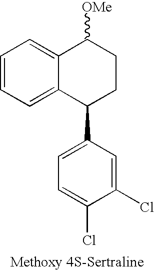

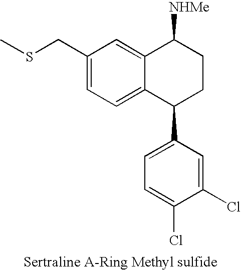

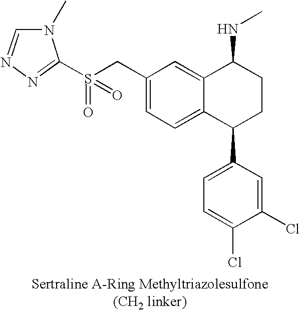

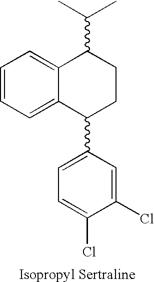

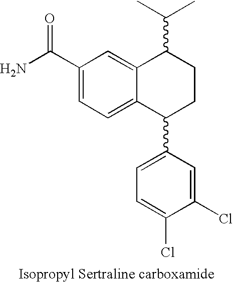

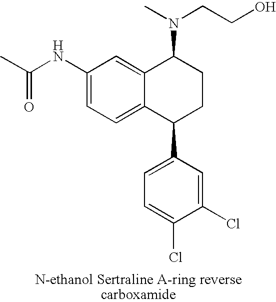

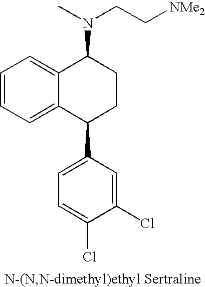

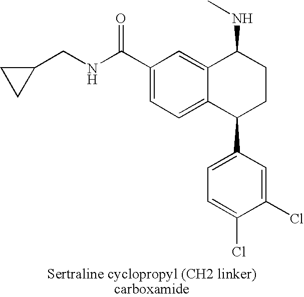

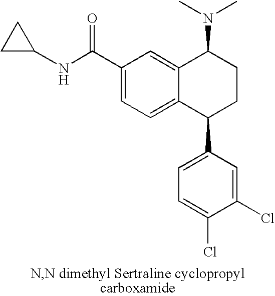

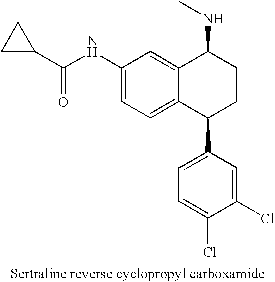

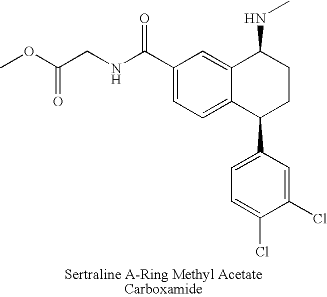

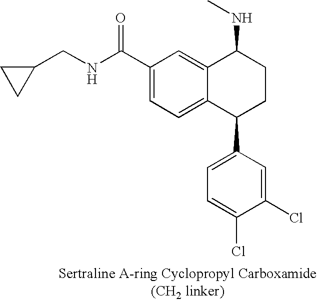

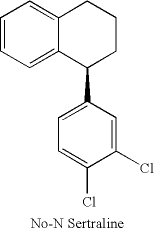

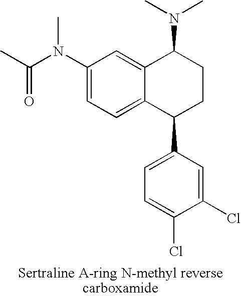

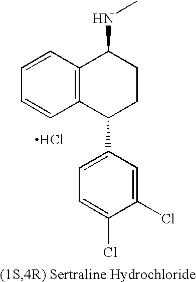

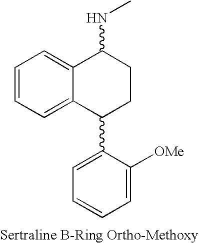

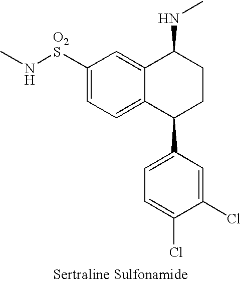

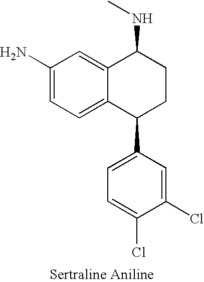

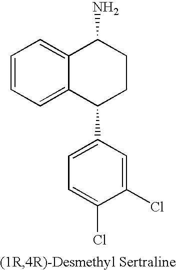

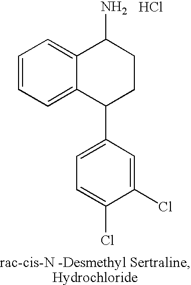

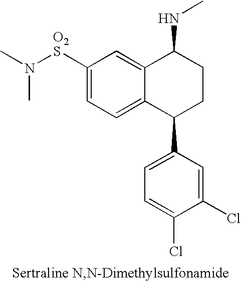

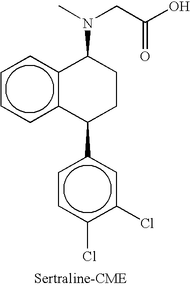

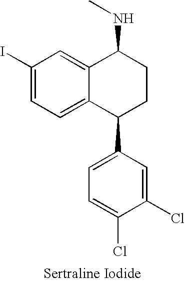

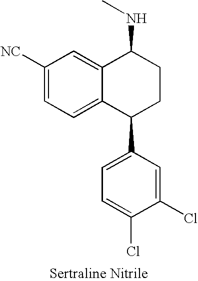

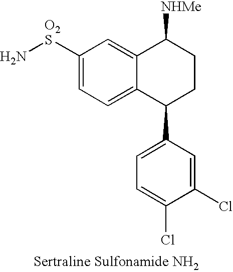

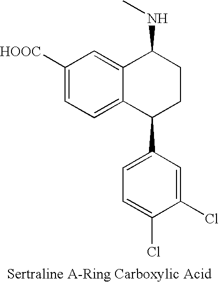

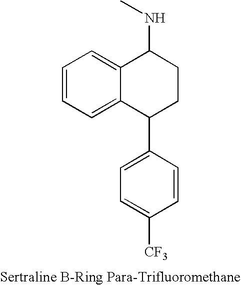

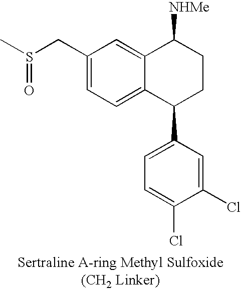

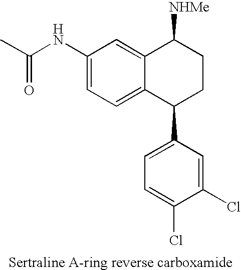

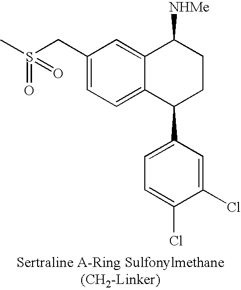

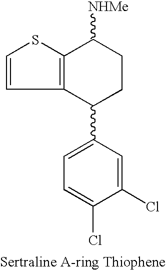

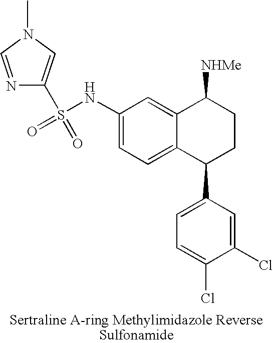

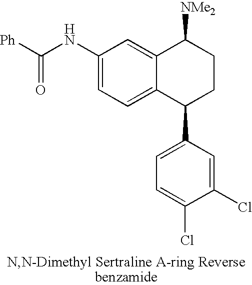

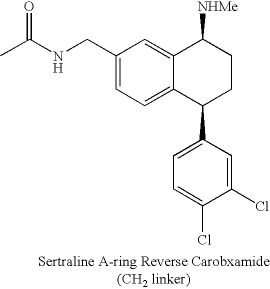

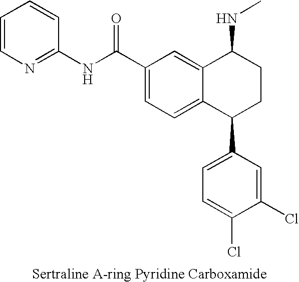

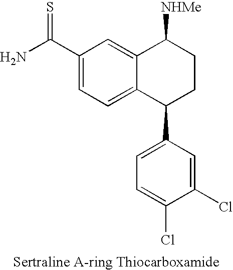

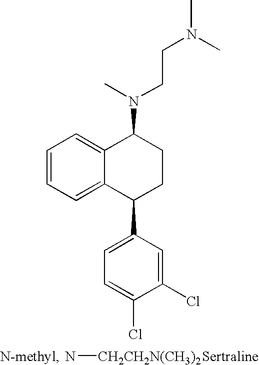

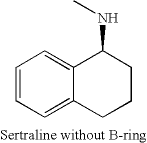

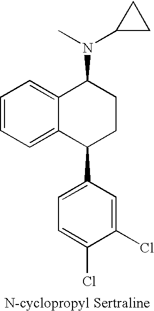

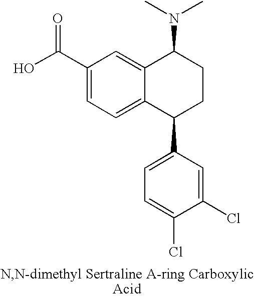

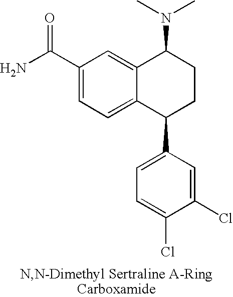

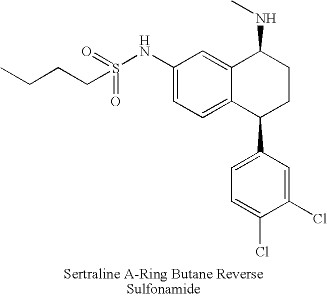

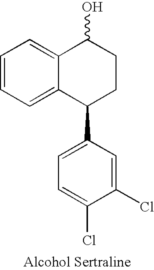

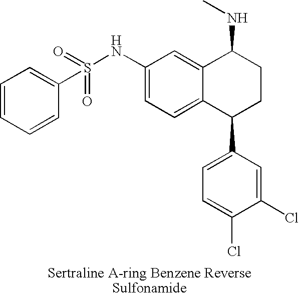

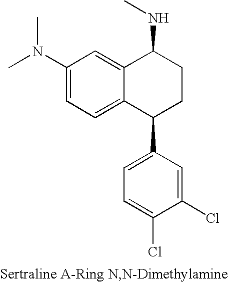

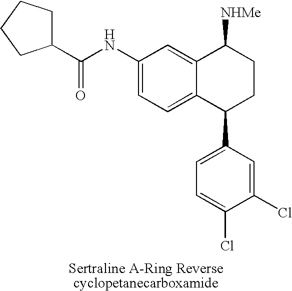

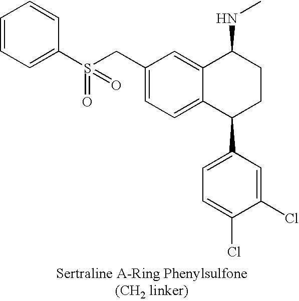

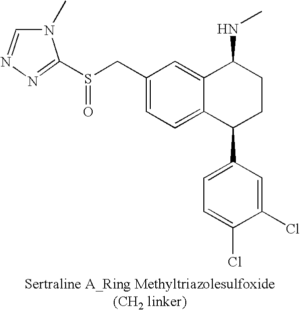

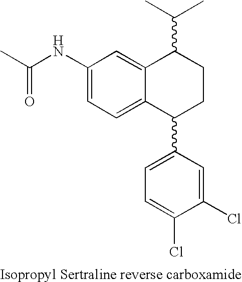

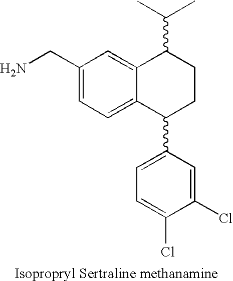

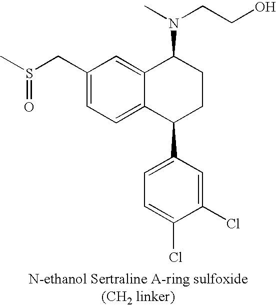

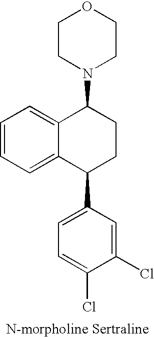

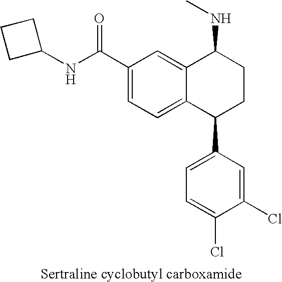

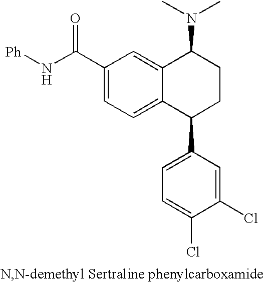

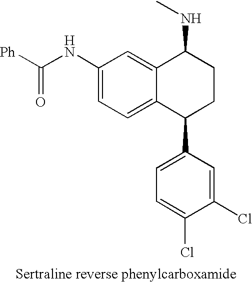

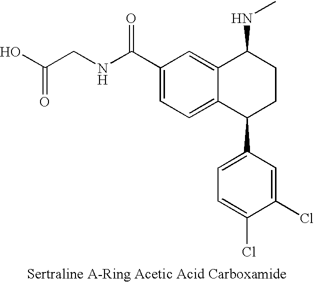

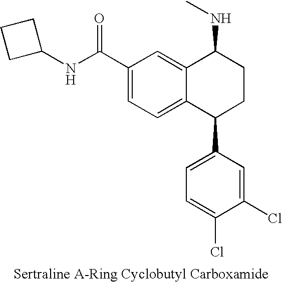

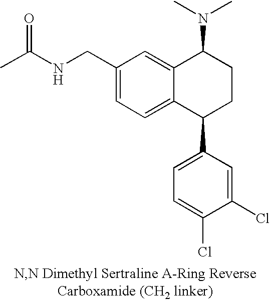

Particularly useful are the following compounds, in either the (1S)-enantiomeric or (1S)(1R) racemic forms, and their pharmaceutically acceptable salts: cis-N-methyl-4-(3,4-dichlorophenyl)-1,2,3,4-tetrahydro-1-naphthalenamine; cis-N-methyl-4-(4-bromophenyl)-1,2,3,4-tetrahydro-1-naphthalenamine; cis-N-methyl-4-(4-chlorophenyl)-1,2,3,4-tetrahydro-1-naphthalenamine; cis-N-methyl-4-(3-trifluoromethyl-phenyl)-1,2,3,4-tetrahydro-1-naphthalenamine; cis-N-methyl-4-(3-trifluoromethyl-4-chlorophenyl)-1,2,3,4-tetrahydro-1-naphthalenamine; cis-N,N-dimethyl-4-(4-chlorophenyl)-1,2,3,4-tetrahydro-1-naphthalenamine; cis-N,N-dimethyl-4-(3-trifluoromethyl-phenyl)-1,2,3,4-tetrahydro-1-naphthalenamine; and cis-N-methyl-4-(4-chlorophenyl)-7-chloro-1,2,3,4-tetrahydro-1-naphthalenamine. Of interest also is the (1R)-enantiomer of cis-N-methyl-4-(3,4-dichlorophenyl)-1,2,3,4-tetrahydro-1-naphthalenamine.

### UK-416244

MC-416244 is an SSRI that is phenoxybenzylamine derivative. UK-416244 has the structure:

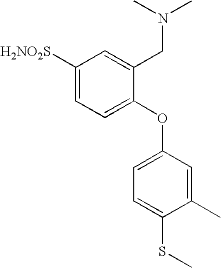

Structural analogs of UK-416244 are compounds having the formula:

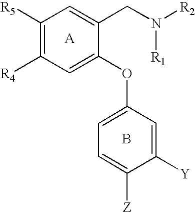

where R₁ and R₂, independently, are H, C₁₋₆ alkyl (e.g., CH₃) or substituted heteroalkyl, or (CH₂)_{d}(C₃₋₆ cycloalkyl) where d is 0, 1, 2, or 3; or R₁ and R₂ together with the nitrogen to which they are attached form an azetidine ring; Z or Y is —S(O)ₙR₃ and the other Z or Y is halogen or —R₃; where R₃ is independently C₁₋₄ alkyl optionally substituted with fluorine (e.g., where R₃ is or is not CF₃) and n is 0, 1, or 2; or Z and Y are linked so that, together with the interconnecting atoms, Z and Y form a fused 5 to 7-membered carbocyclic or heterocyclic ring which may be saturated, unsaturated, or aromatic, and where when Z and Y form a heterocyclic ring, in addition to carbon atoms, the linkage contains one or two heteroatoms independently selected from O, S, and N; (e.g., with the proviso that when R₅ is F and R₂ is methyl then the fused ring is not 1,3-dioxolane and Z and Y together do not form a fused phenyl ring); R₄ and R₅ are, independently, A-X, where A is —CH═CH— or —(CH₂)ₚ— where p is 0, 1, or 2; X is H, F, Cl, Br, I, NH₂, OH, CONR₆R₇, SO₂NR₆R₇, SO₂NHC(═O)R₆, C₁₋₄ alkoxy, NR₈SO₂R₉, NO₂, NR₆R₁₁ (e.g., N(CH₃)₂, CN, CO₂R₁₀ (e.g., COOH), CHO, SR₁₀, S(O)R₉ or SO₂R₁₀; R₆, R₇, R₈ and R₁₀ independently are H, C₁₋₆ alkyl (e.g., CH₃, (CH₂)₃CH₃ or cyclopropyl), C₆₋₁₂ aryl (e.g., phenyl) optionally substituted independently by one or more R₁₂, or C₁₋₆ alkyl-aryl optionally substituted (e.g., CH₂Ph); R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl, or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a 4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl), or —N(C₁₋₆ alkyl)₂—;

or compounds having the formula:

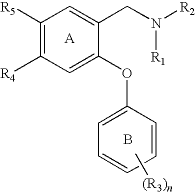

where R₁ and R₂ are independently H, C₁₋₆ alkyl (e.g., CH₃) or substituted heteroalkyl, (CH₂)ₘ(C₃₋₆ cycloalkyl) where m is 0, 1, 2, or 3, or R₁ and R₂ together with the nitrogen to which they are attached form an azetidine ring; each R₃ is independently H, I, Br, F, Cl, C₁₋₆ alkyl (e.g., CH₃), CF₃, CN, OCF₃, C₁₋₄ alkylthio (e.g., SCH₃), C₁₋₄ alkoxy (e.g., OCH₃), aryloxy (e.g., OPh), or CONR₆R₇; n is 1, 2, or 3; and R₄ and R₅ are independently A-X, where A is —CH═CH— or —(CH₂)ₚ— where p is 0, 1, or 2; X is H, F, Cl, Br, I, CONR₆R₇, SO₂NR₆R₇, SO₂NHC(═O)R₆, OH, C₁₋₄ alkoxy, NR₈SO₂R₉, NO₂, NR₆R₁₁, CN, CO₂R₁₀ (e.g., COOH), CHO, SR₁₀, S(O)R₉, or SO₂R₁₀; R₆, R₇, R₈, and R₁₀ are independently H or C₁₋₆ alkyl (e.g., (CH₂)₃CH₃ or cyclopropyl), C₆₋₁₂ aryl (e.g., phenyl) optionally substituted independently by one or more R₁₂, or C₁₋₆ alkyl-aryl optionally substituted; R₉ is C₁₋₆ alkyl optionally substituted independently by one or more R₁₂; R₁₁ is H, C₁₋₆ alkyl optionally substituted independently by one or more R₁₂, C(O)R₆, CO₂R₉, C(O)NHR₆, or SO₂NR₆R₇; R₁₂ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₁₃; or R₆ and R₇, together with the nitrogen to which they are attached, form a:4-, 5-, or 6-membered heterocyclic ring optionally substituted independently by one or more R₁₃; or a 5- or 6-membered heterocyclic ring containing 1, 2, or 3 heteroatoms selected from N, S, and O optionally substituted independently by one or more R₁₃; where R₁₃ is hydroxy, C₁₋₄ alkoxy, F, C₁₋₆ alkyl, haloalkyl, haloalkoxy, —NH₂, —NH(C₁₋₆ alkyl) or -N(C₁₋₆ alkyl)₂ (e.g., where when R₁ and R₂ are methyl, R₄ and R₅ are hydrogen and n is 1, R₃ is not a —SMe group para to the ether linkage linking rings A and B). In certain embodiments, n is 1 or 2, and the R₃ group(s) is/are at positions 3 and/or 4 of the B ring, for example, are CH₃, SCH₃, OCH₃, Br, or CF₃. For either of the above structures, R₄ or R₅ can be SO₂NHPh, SO₂NHCH₃, CN, H, Br, CONH₂, COOH, SO₂NHCH₂Ph, SO₂NHCOCH₃, CH₂NHSO₂CH₃NH₂, OR NO₂, benzyl amide, acylsulfonamide, reverse sulfonamide, NHCH₃, N(CH₃)₂, SO₂NH₂, CH₂OH, NHSO₂CH₃, SO₂NHCH₂CCH₂, CH₂NH₂, SO₂NHBu, and SO₂NHcyclopropyl. UK-416244 structural analogs are described in U.S. Pat. Nos. 6,448,293 and 6,610,747. UK-416244 analogs are described below.

Other analogs of UK-416244 can be described by the formula:

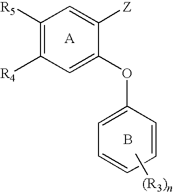

where are R₃, R₄, and R₅ are as defined above and Z is CH₂NR₁R₂ where R₁ and R₂ are as defined above, C₁₋₆ alkyl, optionally substituted (e.g., with hydroxyl, NH₂, NHC₁₋₆ alkyl). In certain embodiments, Z is CH₂CH(CH₃)₂, CH₂OCH₃, CH₂N(CH₃)CH₂CH₂OH, N(CH₃)₂, CH₂N(CH₃)₂, COOH, CH₂NHCH₃, CH₂OH, CH₂NHCOCH₃, or CONHCH₃.

Other UK-416244 analogs are described by the formula.

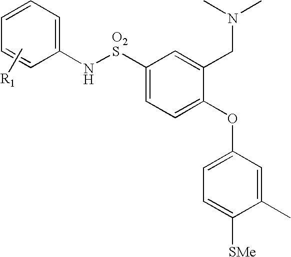

where R₁ is H, I, Br, F, Cl, C₁₋₆ alkyl (e.g., CH₃), CF₃, CN, OCF₃, C₁₋₄ alkylthio (e.g., SCH₃), C₁₋₄ alkoxy (e.g., OCH₃), aryloxy, or CONR₂R₃; n is 1, 2, or 3; R₂ and R₃ are independently H or C₁₋₆ alkyl (e.g., (CH₂)₃CH₃ or cyclopropyl), C₆₋₁₂ aryl (e.g., phenyl) optionally substituted independently by one or more R₄, or C₁₋₆ alkyl-aryl optionally substituted; R₄ is F (preferably up to 3), OH, CO₂H, C₃₋₆ cycloalkyl, NH₂, CONH₂, C₁₋₆ alkoxy, C₁₋₆ alkoxycarbonyl or a 5- or 6-membered heterocyclic ring containing 1, 2, or 3 heteroatoms selected from N, S, and O optionally substituted independently by one or more R₅; or R₂ and R₃, together with the nitrogen to which they are attached, form a 4-, 5-, or 6-membered heterocyclic ring optionally substituted independently by one or more R₅; or a 5- or 6-membered heterocyclic ring containing 1, 2, or 3 heteroatoms selected from N, S, and O optionally substituted independently by one or more R₅; where R₅ is hydroxy, C₁₋₄ alkoxy, F, C₁₋₆ alkyl, haloalkyl, haloalkoxy, —NH₂, —NH(C₁₋₆ alkyl) or —N(C₁₋₆ alkyl)₂. In certain embodiments, where R₁ is Br, OMe, NO₂, CO₂Me, or CN. R₁ may be at the ortho, meta, or para position)

Still other MC-416244 analogs are described by the formula:

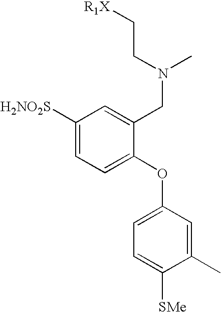

where X is N, O, or S, and R₁ is H, C₁₋₆ alkyl or substituted heteroalkyl, (CH₂)ₘ(C₃₋₆ cycloalkyl) where m is 0, 1, 2, or 3.

Additional compounds have the structure:

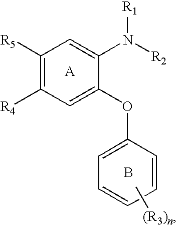

where R₁ is H or C₁₋₆ alkyl (e.g., CH₃, CH₂CH₃) and R₂ is C₁₋₆ alkyl substituted with OH, such as CH₂OH, CH₂CH₂OH, CH(OH)CH₃, CH₂CH₂CH₂OH, CH(CH₂)CH₂OH, and CH₂CH₂CH₂CH₂OH, CH(OH)CH₂CH₂CH₃, CH₂CH(OH)CH₂CH₃, and CH₂CH₂CH(OH)CH₃) or is CH₂XR₁₄ or CH₂CH₂XR₁₄, where X is N, O, or S, and R₁₄ is H, C₁₋₆ alkyl or substituted heteroalkyl, (CH₂)_{q}(C₃₋₆ cycloalkyl) where q is 0, 1, 2, or 3, and where R₃, R₄, and R₅ are as defined above. In certain embodiments, the compound has the structure,

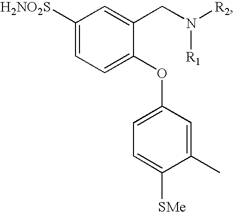

where R₁ is H or C₁₋₆ alkyl (e.g., CH₃, CH₂CH₃) and R₂ is C₁₋₆ alkyl substituted with OH, e.g., CH₂OH, CH₂CH₂OH, CH(OH)CH₃, CH₂CH₂CH₂OH, CH(CH₂)CH₂OH, and CH₂CH₂CH₂CH₂OH, CH(OH)CH₂CH₂CH₃, CH₂CH(OH)CH₂CH₃, and CH₂CH₂CH(OH)CH₃). In particular embodiments, the compound is:

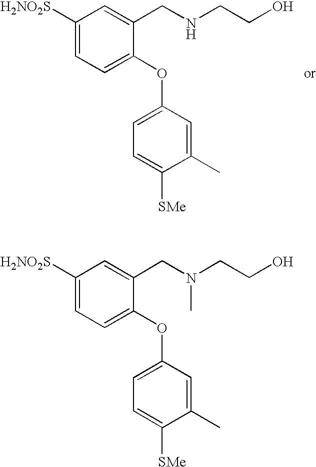

UK-416244 analogs include those of Table 5:

TABLE 5
UK-416244 analogs

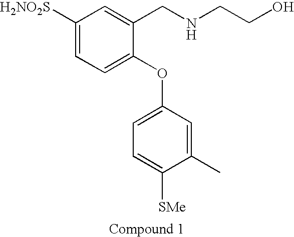

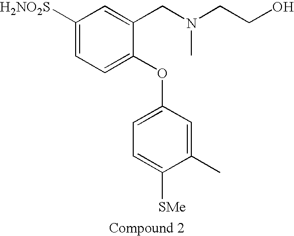

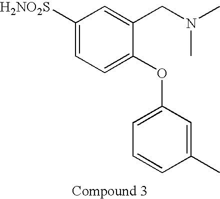

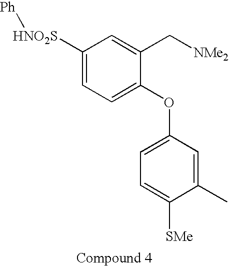

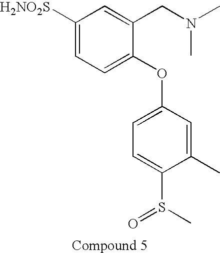

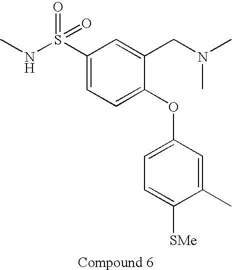

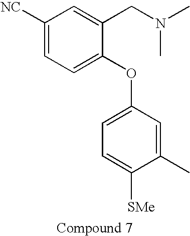

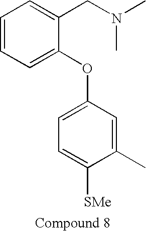

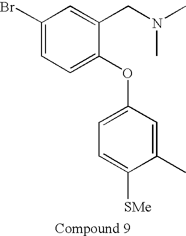

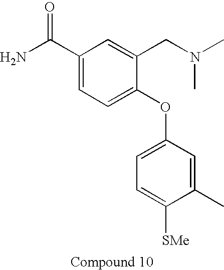

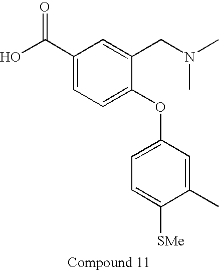

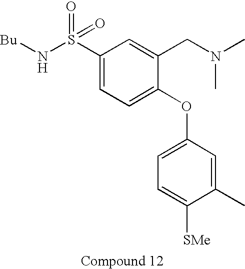

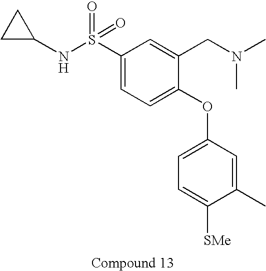

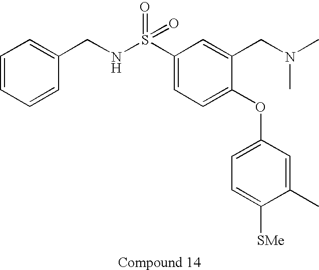

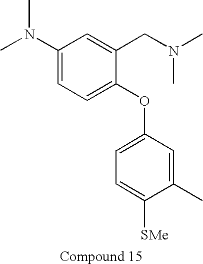

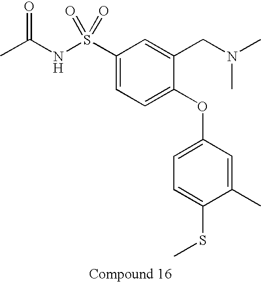

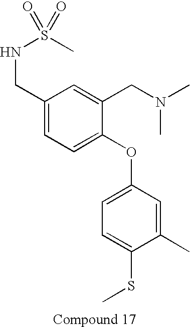

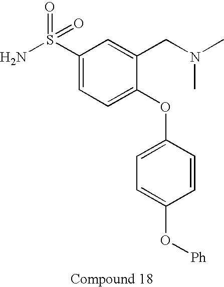

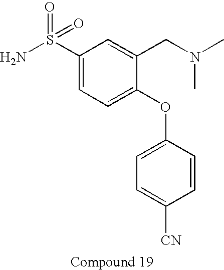

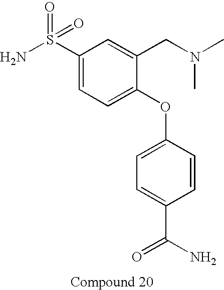

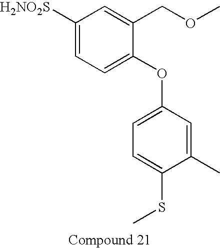

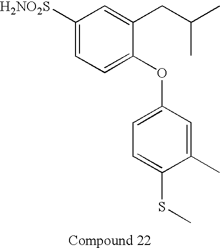

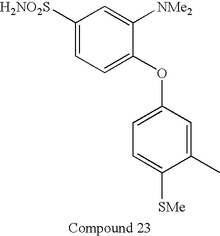

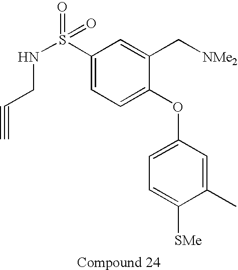

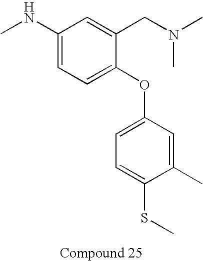

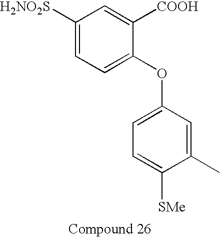

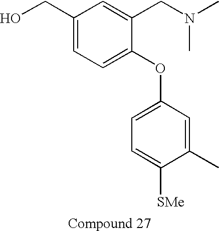

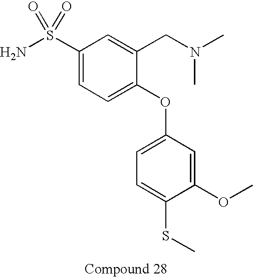

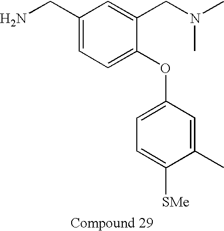

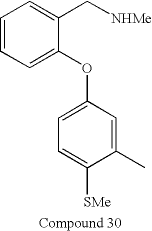

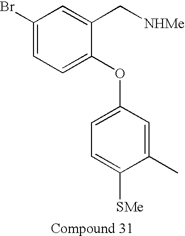

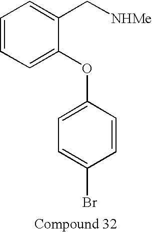

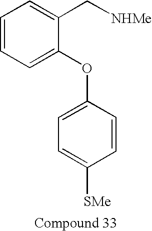

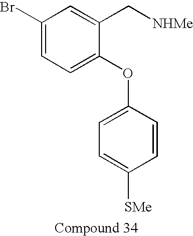

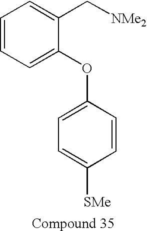

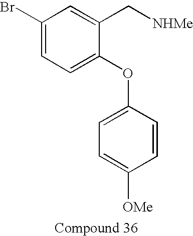

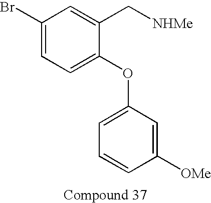

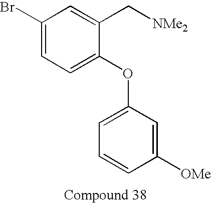

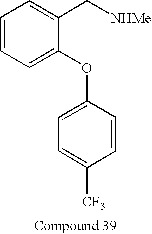

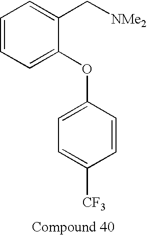

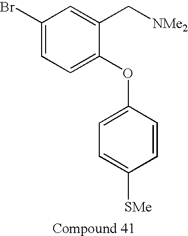

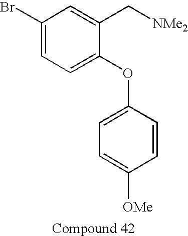

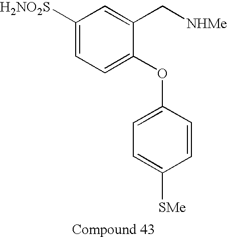

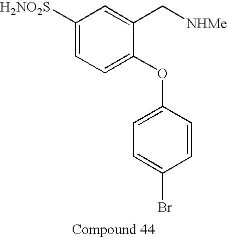

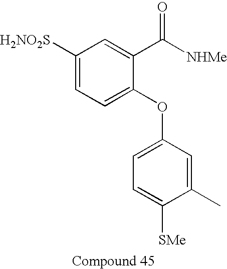

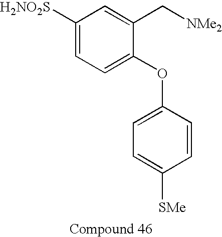

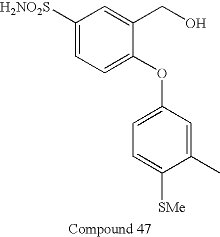

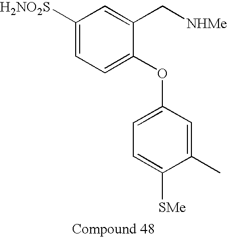

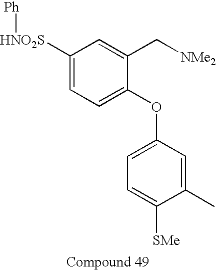

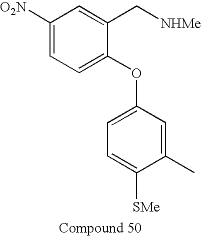

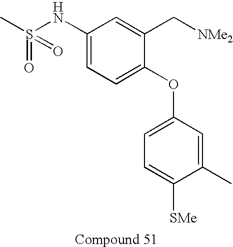

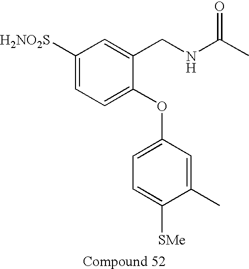

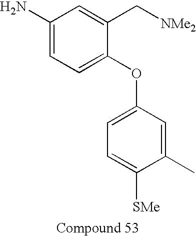

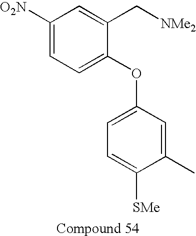

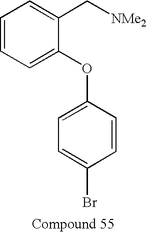

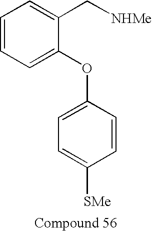

Sertraline, UK-416244, and analogs thereof are considered herein to be equivalents in the methods, compositions, and kits of the invention. The synthesis of certain of the above sertraline, UK-416244, and analogs thereof have been described in co-pending application 61/______, attorney docket no. 50425/004005, entitled “Compositions and Methods for Treatment of Viral Diseases,” filed Aug. 18, 2008.

### Metabolites

Pharmacologically active metabolites of any of the foregoing SSRIs can also be used in the methods, compositions, and kits of the invention. Exemplary metabolites are didesmethylcitalopram, desmethylcitalopram, desmethylsertraline, and norfluoxetine.

Analogs

Functional analogs of SSRIs can also be used in the methods, compositions, and kits of the invention. Exemplary SSRI functional analogs are provided below. One class of SSRI analogs includes SNRIs (selective serotonin norepinephrine reuptake inhibitors), which include venlafaxine, duloxetine, and 4-(2-fluorophenyl)-6-methyl-2-piperazinothieno[2,3-d]pyrimidine.

Structural analogs of venlafaxine are those compounds having the formula:

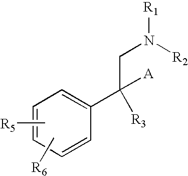

as well as pharmaceutically acceptable salts thereof, wherein A is a moiety of the formula:

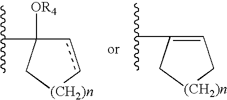

where the dotted line represents optional unsaturation; R₁ is hydrogen or alkyl; R₂ is C₁₋₄ alkyl; R₄ is hydrogen, C₁₋₄ alkyl, formyl or alkanoyl; R₃ is hydrogen or C₁₋₄ alkyl; R₅ and R₆ are, independently, hydrogen, hydroxyl, C₁₋₄ alkyl, C₁₋₄ alkoxy, C₁₋₄ alkanoyloxy, cyano, nitro, alkylmercapto, amino, C₁₋₄ alkylamino, dialkylamino, C₁₋₄ alkanamido, halo, trifluoromethyl or, taken together, methylenedioxy; and n is 0, 1, 2, 3 or 4.

Structural analogs of duloxetine are those compounds described by the formula disclosed in U.S. Pat. No. 4,956,388, hereby incorporated by reference. Other SSRI analogs are 4-(2-fluorophenyl)-6-methyl-2-piperazinothieno[2,3-d]pyrimidine, 1,2,3,4-tetrahydro-N-methyl-4-phenyl-1-naphthylamine hydrochloride; 1,2,3,4-tetrahydro-N-methyl-4-phenyl-(E)-1-naphthylamine hydrochloride; N,N-dimethyl-1-phenyl-1-phthalanpropylamine hydrochloride; gamma-(4-(trifluoromethyl)phenoxy)-benzenepropanamine hydrochloride; BP 554; CP 53261; O-desmethylvenlafaxine; WY 45,818; WY 45,881; N-(3-fluoropropyl)paroxetine; Lu 19005; and SNRIs described in PCT Publication No. WO 04/004734.

### Antiviral Agents

In certain embodiments, an antiviral agent can be used in the compositions, methods, and kits of the invention. Suitable antiviral agents include, without limitation, abacavir, acemannan, acyclovir, adefovir, amantadine, amidinomycin, ampligen, amprenavir, aphidicolin, atevirdine, capravirine cidofovir, cytarabine, delavirdine, didanosine, dideoxyadenosine, n-docosanol, edoxudine, efavirenz, emtricitabine, famciclovir, floxuridine, fomivirsen, foscarnet sodium, ganciclovir, idoxuridine, imiquimod, indinavir, inosine pranobex, interferon-α, interferon-β, kethoxal, lamivudine, lopinavir, lysozyme, madu, methisazone, moroxydine, nelfinavir, nevirapine, nitazoxanide, oseltamivir, palivizumab, penciclovir, enfuvirtide, pleconaril, podophyllotoxin, ribavirin, rimantadine, ritonavir, saquinavir, sorivudine, stallimycin, statolon, stavudine, tenofovir, tremacamra, triciribine, trifluridine, tromantadine, tunicamycin, valacyclovir, valganciclovir, vidarabine, zalcitabine, zanamivir, zidovudine, resiquimod, atazanavir, tipranavir, entecavir, fosamprenavir, merimepodib, docosanol, vx-950, and peg interferon. Additional antiviral agents are listed in Table 2 and Table 3.

Structural analogs of antiviral agents which may be used in the combinations of the invention include 9-((2-aminoethoxy)methyl)guanine, 8-hydroxyacyclovir, 2′-O-glycyl acyclovir, ganciclovir, PD 116124, valacyclovir, omaciclovir, valganciclovir, buciclovir, penciclovir, valmaciclovir, carbovir, theophylline, xanthine, 3-methylguanine, enprofylline, cafaminol, 7-methylxanthine, L 653180, BMS 181164, valomaciclovir stearate, deriphyllin, acyclovir monophosphate, acyclovir diphosphate dimyristoylglycerol, and etofylline.

Edoxudine analogs are described in U.S. Pat. No. 3,553,192. Efavirenz analogs are described in European Patent 582,455 and U.S. Pat. No. 5,519,021. Floxuridine analogs are described in U.S. Pat. Nos. 2,970,139 and 2,949,451. Nelfinavir analogs are described in U.S. Pat. No. 5,484,926. Aphidicolin analogs are described in U.S. Pat. No. 3,761,512. Trifluridine analogs are described in U.S. Pat. No. 3,201,387. Cytarabine analogs are described in U.S. Pat. No. 3,116,282. Triciribine analogs, including triciribine 5′-phosphate and triciribine-dimethylformamide, are described in U.S. Pat. No. 5,633,235. Nitazoxanide analogs are described in U.S. Pat. No. 3,950,391.

Ritonavir

Ritonavir is an antiviral used in treatment of HIV and has the structure:

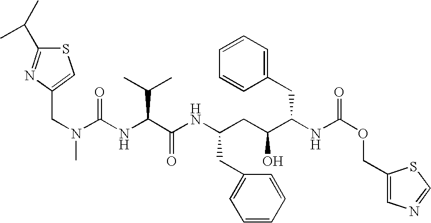

Ritonavir analogs are described, for example, in U.S. Pat. No. 5,541,206 and have the general structure:

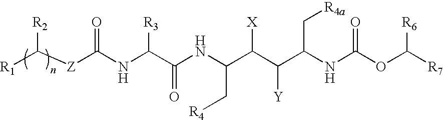

where R₁ is monosubstituted thiazolyl, monosubstituted oxazolyl, monosubstituted isoxazolyl or monosubstituted isothiazolyl wherein the substituent is selected from (i) loweralkyl, (ii) loweralkenyl, (iii) cycloalkyl, (iv) cycloalkylalkyl, (v) cycloalkenyl, (vi) cycloalkenylalkyl, (vii) heterocyclic wherein the heterocyclic is selected from aziridinyl, azetidinyl, pyrrolidinyl, piperidinyl, piperazinyl, morpholinyl, thiomorpholinyl, thiazolyl, oxazolyl, isoxazolyl, isothiazolyl, pyridinyl, pyrimidinyl, pyridazinyl and pyrazinyl and wherein the heterocyclic is unsubstituted or substituted with a substituent selected from halo, loweralkyl, hydroxy, alkoxy and thioalkoxy, (viii) (heterocyclic)alkyl wherein heterocyclic is defined as above, (ix) alkoxyalkyl, (x) thioalkoxyalkyl, (xi) alkylamino, (xii) dialkylamino, (xiii) phenyl wherein the phenyl ring is unsubstituted or substituted with a substituent selected from halo, loweralkyl, hydroxy, alkoxy and thioalkoxy, (xiv) phenylalkyl wherein the phenyl ring is unsubstituted or substituted as defined above, (xv) dialkylaminoalkyl, (xvi) alkoxy and (xvii) thioalkoxy; n is 1,2 or 3; R₂ is hydrogen or loweralkyl; R₃ is loweralkyl; R₄ and R₄ₐ are independently selected from phenyl, thiazolyl and oxazolyl wherein the phenyl, thiazolyl or oxazolyl ring is unsubstituted or substituted with a substituent selected from (i) halo, (ii) loweralkyl, (iii) hydroxy, (iv) alkoxy and (v) thioalkoxy; R₆ is hydrogen or loweralkyl; R₇ is thiazolyl, oxazolyl, isoxazolyl or isothiazolyl wherein the thiazolyl, oxazolyl, isoxazolyl or isothiazolyl ring is unsubstituted or substituted with loweralkyl; X is hydrogen and Y is —OH or X is —OH and Y is hydrogen, with the proviso that X is hydrogen and Y is —OH when Z is —N(R₈)— and R₇ is unsubstituted and with the proviso that X is hydrogen and Y is —OH when R₃ is methyl and R₇ is unsubstituted; and Z is absent, —O—, —S—, —CH₂— or —N(R₈)— wherein R₈ is loweralkyl, cycloalkyl, —OH or —NHR₈ₐ wherein R₈ₐ is hydrogen, loweralkyl or an N-protecting group.

### Telaprevir

In certain embodiments, telaprevir or an analog thereof can be used in the compositions, methods, and kits of the invention. Telaprevir (VX-950) is a hepatitis C therapy. The structure of telaprevir is:

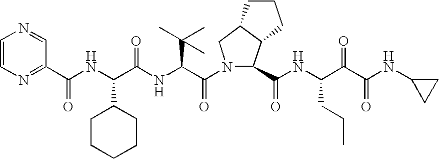

Analogs of telaprevir are described, for example, in U.S. Pat. Application Publication No. 2005/0197299 and can be represented as follows:

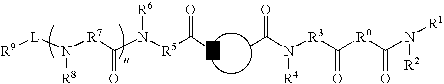

wherein R⁰ is a bond or difluoromethylene; R¹ is hydrogen, optionally substituted aliphatic group, optionally substituted cyclic group or optionally substituted aromatic group; R² and R⁹ are each independently optionally substituted aliphatic group, optionally substituted cyclic group or optionally substituted aromatic group; R³, R⁵, and R⁷ are each independently (optionally substituted aliphatic group, optionally substituted cyclic group or optionally substituted aromatic group)(optionally substituted methylene or optionally substituted ethylene), optionally substituted (1,1- or 1,2-)cycloalkylene or optionally substituted (1,1- or 1,2-)heterocyclylene; R⁴, R⁶, R⁸ and R¹⁰ are each independently hydrogen or optionally substituted aliphatic group;

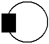

is substituted monocyclic azaheterocyclyl or optionally substituted multicyclic azaheterocyclyl, or optionally substituted multicyclic azaheterocyclenyl wherein the unsaturatation is in the ring distal to the ring bearing the R⁹-L-N(R⁸)—R⁷—C(O)—)ₙN(R⁶)—R⁵—C(O)—N moiety and to which the —C(O)—N(R⁴)—R³—C(O)—C(O)NR²R¹ moiety is attached; L is —C(O)—, —OC(O)—, —NR¹⁰C(O)—, —S(O)₂—, or —NR¹⁰S(O)₂—; and n is 0 or 1, or a pharmaceutically acceptable salt or prodrug thereof, or a solvate of such a compound, its salt or its prodrug, provided when

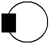

is substituted

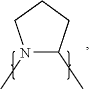

then L is —OC(O)— and R⁹ is optionally substituted aliphatic, or at least one of R³, R⁵ and R⁷ is (optionally substituted aliphatic group, optionally substituted cyclic group or optionally substituted aromatic group)(optionally substituted ethanediyl), or R⁴is optionally substituted aliphatic.

### HCV-796

In certain embodiments, HCV-796 or an analog thereof can be used in the compositions, methods, and kits of the invention. HCV-796 is a non-nucleoside polymerase inhibitor. The structure of HCV-796 is:

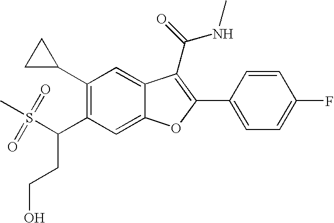

Analogs of HCV-796 are described for example, in U.S. Pat. No. 7,265,152 and have the general structure:

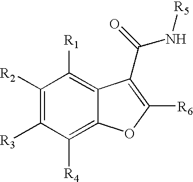

wherein R₁ represents a radical selected from the group consisting of hydrogen, alkyl, halogen, and cyano; R₂ represents a radical selected from the group consisting of hydrogen, a substituted or unsubstituted alkyl radical, a substituted or unsubstituted alkoxy group, hydroxy, cycloalkyl, cycloalkyloxy, polyfluoroalkyl, polyfluoroalkoxy, halogen, amino, monoalkylamino, dialkylamino, cyano, a substituted or unsubstituted benzyloxy group, and a substituted or unsubstituted heterocyclic radical; R₃ represents a radical selected from the group consisting of hydrogen, a substituted or unsubstituted alkyl radical, a substituted or unsubstituted alkoxy group, alkenyl, halogen, hydroxy, polyfluoroalkyl, polyfluoroalkoxy, formyl, carboxyl, alkylcarbonyl, alkoxycarbonyl, hydroxyalkylcarbonyl, amino, a substituted or unsubstituted monoalkylamino, dialkylamino, cyano, amido, alkoxyamido, a substituted or unsubstituted heteroarylamino, acetylsulfonylamino, ureido, carboxamide, sulfonamide, a substituted sulfonamide, a substituted or unsubstituted heterocyclosulfonyl, alkylthio, alkylsulfinyl, alkylsulfonyl, alkylsulfonic acid, a substituted or unsubstituted heterocyclic radical, and —O(CH₂)—C(═O)—R₇; R₄ represents a radical selected from the group consisting of hydrogen, alkyl, halogen, and alkoxy; R₅ represents a radical selected from the group consisting of an alkyl (C₁-C₆) group, cycloalkyl, and cycloalkylalkyl; R₆ represents a radical selected from the group consisting of a substituted or unsubstituted aryl group and a substituted or unsubstituted heteroaryl group; R₇ represents a radical selected from the group consisting of dialkylamino, a substituted or unsubstituted arylamino, a substituted or unsubstituted heteroarylamino, and a substituted or unsubstituted aryl group, said monoalkylamino substituents being one or more radical(s) independently selected from the group consisting of cycloalkyl, hydroxy, alkoxy, and a substituted or unsubstituted heterocyclic radical; said arylamino substituents and said heteroarylamino substituents being one or more radical(s) independently selected from an alkyl group and an alkoxycarbonyl; said sulfonamide substituents being one or more radical(s) independently selected from the group consisting of alkenyl, cycloalkyl, alkoxy, hydroxy, halogen, polyfluoroalkyl, polyfluoroalkoxy, carboxyl, alkylcarbonyl, alkoxycarbonyl, carboxamide, a substituted or unsubstituted aryl group, and a substituted or unsubstituted heterocyclic radical; said heterocyclosulfonyl substituents being one or more radical(s) independently selected from the group consisting of alkoxy and hydroxy; said alkyl radical substituents and said alkoxy group substituents being one or more radical(s) independently selected from the group consisting of alkenyl, amino, monoalkylamino, dialkylamino, alkoxy, cycloalkyl, hydroxy, carboxyl, halogen, cyano, polyfluoroalkyl, polyfluoroalkoxy, sulfonamide, carboxamide, alkylsulfonyl, alkylcarbonyl, alkoxycarbonyl, mercapto, 2,2-dimethyl-4-oxo-4H-benzo[1,3]dioxinyl, a substituted or unsubstituted aryl group, and a substituted or unsubstituted heterocyclic radical; said heterocyclic radical substituents being one or more radical(s) independently selected from the group consisting of alkyl, amino, amido, monoalkylamino, cycloalkyl-alkylamino, dialkylamino, alkoxy, alkoxyalkyl, hydroxy, hydroxyalkyl, cycloalkyl, cycloalkylalkyl, carboxyl, carboxamide, halogen, haloalkyl, cyano, polyfluoroalkyl, polyfluoroalkoxy, alkylsulfonyl, alkylcarbonyl, cycloalkylcarbonyl, alkoxycarbonyl, mercapto, oxo, a substituted or unsubstituted aryl group, arylalkyl, and a substituted or unsubstituted heteroaryl group; said heteroaryl group substituents being one or more radical(s) independently selected from the group consisting of alkyl, amino, alkoxy, alkoxyalkyl, hydroxy, hydroxyalkyl, cycloalkyl, carboxyl, carboxamide, halogen, polyfluoroalkyl, polyfluoroalkoxy, alkylsulfonyl, mercapto, and oxo; said benzyloxy group substituents being one or more radical(s) independently selected from the group consisting of alkyl, alkoxy, polyfluoroalkyl, polyfluoroalkoxy, hydroxy, carboxyl, alkoxycarbonyl, halogen, cyano, alkylsulfonyl, and phenyl; said aryl group substituents being one or more radical(s) independently selected from the group consisting of alkyl, acetylenyl, alkoxy, hydroxy, halogen, polyfluoroalkyl, polyfluoroalkoxy, cyano, amino, monoalkylamino, dialkylamino, aminoalkyl, alkoxyalkoxy, amido, amidoalkyl, carboxyl, alkylsulfonyl, alkylcarbonyl, alkoxycarbonyl, mercapto, and a heterocyclic radical; and pharmaceutically acceptable salts thereof;

### Merimepodib (VX-497)

In certain embodiments, merimepodib or an analog thereof can be used in the compositions, methods, and kits of the invention. Merimepodib is an inhibitor of inosine-5′-monophosphate dehydrogenase (IMPDH) and is used to treat HCV. The structure of merimepodib is:

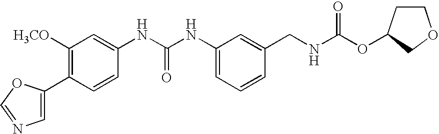

Analogs of merimepodib are described for example, in U.S. Pat. No. 6,541,496 and have the general structure:

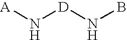

wherein A is selected from (C₁-C₆)-straight or branched alkyl, or (C₂-C₆)-straight or branched alkenyl or alkynyl; and A optionally comprises up to 2 substituents, wherein the first of said substituents, if present, is selected from R¹ or R³, and the second of said substituents, if present, is R¹; B is a saturated, unsaturated or partially saturated monocyclic or bicyclic ring system optionally comprising up to 4 heteroatoms selected from N, O, or S and selected from the formulae:

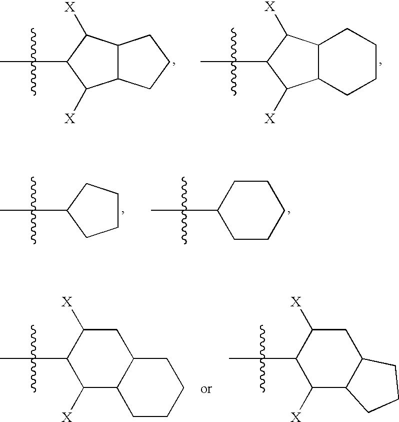

wherein each X is the number of hydrogen atoms necessary to complete proper valence; and B optionally comprises up to 3 substituents, wherein: the first of said substituents, if present, is selected from R¹, R², R⁴ or R⁵, the second of said substituents, if present, is selected from R¹ or R⁴, and the third of said substituents, if present, is R¹; and D is selected from C(O), C(S), or S(O)₂; wherein each R¹ is independently selected from 1,2-methylenedioxy, 1,2-ethylenedioxy, R⁶ or (CH₂)—Y; wherein n is 0, 1 or 2; and Y is selected from halogen, CN, NO₂, CF₃, OCF₃, OH, SR⁶, S(O)R⁶, SO₂R⁶, NH₂, NHR⁶, N(R⁶)₂, NR⁶R⁸, COOH, COOR⁶ or OR⁶; each R² is independently selected from (C₁-C₄)-straight or branched alkyl, or (C₂-C₄)-straight or branched alkenyl or alkynyl; and each R² optionally comprises up to 2 substituents, wherein the first of said substituents, if present, is selected from R¹, R⁴ and R⁵, and the second of said substituents, if present, is R¹; R³ is selected from a monocyclic or a bicyclic ring system consisting of 5 to 6 members per ring, wherein said ring system optionally comprises up to 4 heteroatoms selected from N, O, or S, and wherein a CH₂ adjacent to any of said N, O, or S heteroatoms is optionally substituted with C(O); and each R³ optionally comprises up to 3 substituents, wherein the first of said substituents, if present, is selected from R¹, R², R⁴ or R⁵, the second of said substituents, if present, is selected from R¹ or R⁴, and the third of said substituents, if present, is R¹; each R⁴ is independently selected from OR⁵, OC(O)R⁶, OC(O)R⁵, OC(O)OR⁶, OC(O)OR⁵, OC(O)N(R⁶)₂, OP(O)(OR⁶)₂, SR⁶, SR⁵, S(O)R⁶, S(O)R⁵, SO₂R⁶, SO₂R⁵, SO₂N(R⁶)₂, SO₂NR⁵R⁶, SO₃R⁶, C(O)R⁵, C(O)OR⁵, C(O)R⁶, C(O)OR⁶, NC(O)C(O)R⁶, NC(O)C(O)R⁵, NC(O)C(O)OR⁶, NC(O)C(O)N(R⁶)₂, C(O)N(R⁶)₂, C(O)N(OR⁶)R⁶, C(O)N(OR⁶)R⁵, C(NOR⁶)R⁶, C(NOR⁶)R⁵, N(R⁶)₂, NR⁶C(O)R¹, NR⁶C(O)R⁶, NR⁶C(O)OR⁵, NR⁶C(O)OR⁶, NR⁶C(O)OR⁵, NR⁶C(O)N(R⁶)₂, NR⁶C(O)NR⁵R⁶, NR⁶SO₂R⁶, NR⁶SO₂R⁵, NR⁶SO₂N(R⁶)₂, NR⁶SO₂NR⁵R⁶, N(OR⁶)R⁶, N(OR⁶)R⁵, P(O)(OR⁶)N(R⁶)₂, and P(O)(OR⁶)₂; each R⁵ is a monocyclic or a bicyclic ring system consisting of 5 to 6 members per ring, wherein said ring system optionally comprises up to 4 heteroatoms selected from N, O, or S, and wherein a CH₂ adjacent to said N, O or S maybe substituted with C(O); and each R⁵ optionally comprises up to 3 substituents, each of which, if present, is R¹; each R⁶ is independently selected from H, (C₁-C₄)-straight or branched alkyl, or (C₂-C₄) straight or branched alkenyl; and each R⁶ optionally comprises a substituent that is R⁷; R⁷ is a monocyclic or a bicyclic ring system consisting of 5 to 6 members per ring, wherein said ring system optionally comprises up to 4 heteroatoms selected from N, O, or S, and wherein a CH₂ adjacent to said N, O or S maybe substituted with C(O); and each R⁷ optionally comprises up to 2 substituents independently chosen from H, (C₁-C₄)-straight or branched alkyl, (C₂-C₄) straight or branched alkenyl, 1,2-methylenedioxy, 1,2-ethylenedioxy, or (CH₂)ₙ—Z; wherein n is 0, 1 or 2; and Z is selected from halogen, CN, NO₂, CF₃, OCF₃, OH, S(C₁-C₄)-alkyl, SO(C₁-C₄)-alkyl, SO₂(C₁-C₄)-alkyl, NH₂, NH(C₁-C₄)-alkyl)₂, N((C₁-C₄)-alkyl)R⁸, COOH, C(O)O(C₁-C₄)-alkyl or O(C₁-C₄)-alkyl; and R⁸ is an amino protecting group; and wherein any carbon atom in any A, R² or R⁶ is optionally replaced by O, S, SO, SO₂, NH, or N(C₁-C₄)-alkyl.

### Valopicitabine

In certain embodiments, valopicitabine (NM-283) or an analog thereof can be used in the compositions, methods, and kits of the invention. Valopicitabine is a hepatitis C therapy that acts as a polymerase inhibitor. Valopicitabine is an orally available prodrug of 2′-C-methylcytidine. The structure of valopicitabine is:

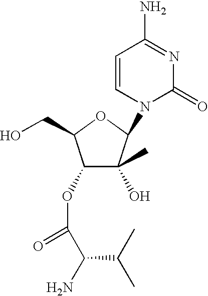

Analogs of valopicitabine are described, for example, in U.S. Pat. Application Publication No. 2007/0015905, which is hereby incorporated by reference.

### Boceprevir (SCH 503034)

In certain embodiments, boceprevir (SCH 503034) or an analog thereof can be used in the compositions, methods, and kits of the invention. Boceprevir is a hepatitis C therapy that acts as a inhibitor of the NS3-serine protease. The structure of boceprevir is:

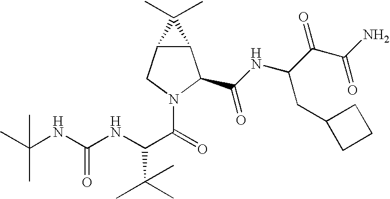

Analogs of boceprivir are described, for example, in U.S. Pat. Application Publication No. 2004/0254117 and have the general structure:

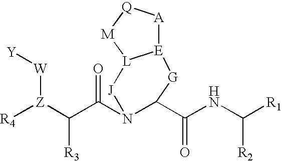

wherein Y is selected from the group consisting of the following moieties: alkyl, alkyl-aryl, heteroalkyl, heteroaryl, aryl-heteroaryl, alkyl-heteroaryl, cycloalkyl, alkyloxy, alkyl-aryloxy, aryloxy, heteroaryloxy, heterocycloalkyloxy, cycloalkyloxy, alkylamino, arylamino, alkyl-arylamino, arylamino, heteroarylamino, cycloalkylamino and heterocycloalkylamino, with the proviso that Y may be optionally substituted with X₁₁ or X₁₂; X₁₁ is alkyl, alkenyl, alkynyl, cycloalkyl, cycloalkyl-alkyl, heterocyclyl, heterocyclylalkyl, aryl, alkylaryl, arylalkyl, heteroaryl, alkylheteroaryl, or heteroarylalkyl, with the proviso that X₁₁ may be additionally optionally substituted with X₁₂; X₁₂ is hydroxy, alkoxy, aryloxy, thio, alkylthio, arylthio, amino, alkylamino, arylamino, alkylsulfonyl, arylsulfonyl, alkylsulfonamido, arylsulfonamido, carboxy, carbalkoxy, carboxamido, alkoxycarbonylamino, alkoxycarbonyloxy, alkylureido, arylureido, halogen, cyano, or nitro, with the proviso that said alkyl, alkoxy, and aryl may be additionally optionally substituted with moieties independently selected from X₁₂; R₁ is COR₅ or B(OR)₂, wherein R₅ is H, OH, OR₈, NR₉R₁₀, CF₃, C₂F₅, C₃F₇, CF₂R₆, R₆, or COR₇ wherein R₇ is H, OH, OR₈, CHR₉R₁₀, or NR₉R₁₀, wherein R₆, R₈, R₉ and R₁₀ are independently selected from the group consisting of H, alkyl, aryl, heteroalkyl, heteroaryl, cycloalkyl, cycloalkyl, arylalkyl, heteroarylalkyl, [CH(R₁′)]ₚCOOR₁₁, [CH(R₁′)]ₚCONR₁₂R₁₃, [CH(R₁′)]ₚSO₂R₁₁, [CH(R₁′)]ₚCOR₁₁, [CH(R₁′)]ₚCH(OH)R₁₁, CH(R₁′)CONHCH(R₂′)COOR₁₁, CH(R₁′)CONHCH(R₂′)CON—R₁₂R₁₃, CH(R₁′)CONHCH(R₂′)R₁₁, CH(R₁′)CONHCH(R₂′)CONHCH(R₃′)COOR₁₁, CH(R₁′)CONHCH(R₂′)CONHCH(R₃′)CONR₁₂R₁₃, CH(R₁′)CONHCH(R₂′)CONHCH(R₃′)CONHCH(R₄′)COOR₁₁, CH(R₁′)CONHCH(R₂′)CONHCH(R₃′)CONHCH(R₄′)CONR₁₂R.— sup.13, CH(R₁′)CONHCH(R₂′)CONHCH(R₃′)CONHCH(R₄′)CONHCH—(R₅′)COOR₁₁ and CH(R₁′)CONHCH(R₂′)CONHCH(R₃′)CON—HCH(R₄′)CONHCH(R₅′) CONR₁₂R₁₃, wherein R₁′, R₂′, R₃′, R₄′, R₅′, R₁₁, R₁₂, R₁₃, and R′ are independently selected from the group consisting of H, alkyl, aryl, heteroalkyl, heteroaryl, cycloalkyl, alkyl-aryl, alkyl-heteroaryl, aryl-alkyl and heteroaralkyl; Z is selected from O, N; CH or CR; W may be present or absent, and if W is present, W is selected from C═O, C═S, C(═N—CN), or SO₂; Q may be present or absent, and when Q is present, Q is CH, N, P, (CH₂)ₚ, (CHR)ₚ, (CRR′)ₚ, O, NR, S, or SO₂; and when Q is absent, M may be present or absent; when Q and M are absent, A is directly linked to L; A is O, CH₂, (CHR)ₚ, (CHR—CHR′)ₚ, (CRR')ₚ, NR, S, SO₂ or a bond; E is CH, N, CR, or a double bond towards A, L or G; G may be present or absent, and when G is present, G is (CH₂)ₚ, (CHR)ₚ, or (CRR′)ₚ; and when G is absent, J is present and E is directly connected to the carbon atom in Formula I as G is linked to; J maybe present or absent, and when J is present, J is (CH₂)ₚ, (CHR)ₚ, or (CRR′)ₚ, SO₂, NH, NR or O; and when J is absent, G is present and E is directly linked to N shown in Formula I as linked to J; L may be present or absent, and when L is present, L is CH, CR, O, S or NR; and when L is absent, then M may be present or absent; and if M is present with L being absent, then M is directly and independently linked to E, and J is directly and independently linked to E; M may be present or absent, and when M is present, M is O, NR, S, SO₂, (CH₂)ₚ, (CHR)ₚ(CHR—CHR′)ₚ, or (CRR′)ₚ; p is a number from 0 to 6; and R, R′, R₂, R₃ and R₄ are independently selected from the group consisting of H; C₁-C₁₀ alkyl; C₂-C₁₀ alkenyl; C₃-C₈ cycloalkyl; C₃-C₈ heterocycloalkyl, alkoxy, aryloxy, alkylthio, arylthio, amino, amido, ester, carboxylic acid, carbamate, urea, ketone, aldehyde, cyano, nitro, halogen; (cycloalkyl)alkyl and (heterocycloalkyl)alkyl, wherein said cycloalkyl is made of three to eight carbon atoms, and zero to six oxygen, nitrogen, sulfur, or phosphorus atoms, and said alkyl is of one to six carbon atoms; aryl; heteroaryl; alkyl-aryl; and alkyl-heteroaryl; wherein said alkyl, heteroalkyl, alkenyl, heteroalkenyl, aryl, heteroaryl, cycloalkyl and heterocycloalkyl moieties may be optionally and chemically-suitably substituted, with said term “substituted” referring to optional and chemically-suitable substitution with one or more moieties selected from the group consisting of alkyl, alkenyl, alkynyl, aryl, aralkyl, cycloalkyl, heterocyclic, halogen, hydroxy, thio, alkoxy, aryloxy, alkylthio, arylthio, amino, amido, ester, carboxylic acid, carbamate, urea, ketone, aldehyde, cyano, nitro, sulfonamido, sulfoxide, sulfone, sulfonyl urea, hydrazide, and hydroxamate; further wherein said unit N—C-G-E-L-J-N represents a five-membered or six-membered cyclic ring structure with the proviso that when said unit N—C-G-E-L-J-N represents a five-membered cyclic ring structure, or when the bicyclic ring structure in Formula I comprising N, C, G, E, L, J, N, A, Q, and M represents a five-membered cyclic ring structure, then said five-membered cyclic “ring structure lacks a carbonyl group as part of the cyclic ring.

### Interferons

In certain embodiments, an interferon or an analog thereof can be used in the compositions, methods, and kits of the invention. Intefereons includes interferon-α, interferon alfa-2a, interferon alfa-2b, interfereon alfa-2c, interferon alfacon-1, interferon alfa-n1, interferon alfa-n3, inteferon-β, interferon β-1a, interferon β-1b, interferon-γ, interferon γ-1a, interferon γ-1b, and pegylated forms thereof.

### Conjugates

If desired, the agents used in any of the combinations described herein may be covalently attached to one another to form a conjugate of formula I.

(A)-(L)-(B) (I)

In formula I, (A) is a Compound A and (B) is Compound B of a pair of agents from eg., Table 1, and L is a covalent linker that tethers (A) to (B). Conjugates of the invention can be administered to a subject by any route and for the treatment of viral hepatitis (e.g., those described herein).

The conjugates of the invention can be prodrugs, releasing drug (A) and drug (B) upon, for example, cleavage of the conjugate by intracellular and extracellular enzymes (e.g., amidases, esterases, and phosphatases). The conjugates of the invention can also be designed to largely remain intact in vivo, resisting cleavage by intracellular and extracellular enzymes. The degradation of the conjugate in vivo can be controlled by the design of linker (L) and the covalent bonds formed with drug (A) and drug (B) during the synthesis of the conjugate.

Conjugates can be prepared using techniques familiar to those skilled in the art. For example, the conjugates can be prepared using the methods disclosed in G. Hermanson, Bioconjugate Techniques, Academic Press, Inc., 1996. The synthesis of conjugates may involve the selective protection and deprotection of alcohols, amines, ketones, sulfhydryls or carboxyl functional groups of drug (A), the linker, and/or drug (B). For example, commonly used protecting groups for amines include carbamates, such as tert-butyl, benzyl, 2,2,2-trichloroethyl, 2-trimethylsilylethyl, 9-fluorenylmethyl, allyl, and m-nitrophenyl. Other commonly used protecting groups for amines include amides, such as formamides, acetamides, trifluoroacetamides, sulfonamides, trifluoromethanesulfonyl amides, trimethylsilylethanesulfonamides, and tert-butylsulfonyl amides. Examples of commonly used protecting groups for carboxyls include esters, such as methyl, ethyl, tert-butyl, 9-fluorenylmethyl, 2-(trimethylsilyl)ethoxy methyl, benzyl, diphenylmethyl, O-nitrobenzyl, ortho-esters, and halo-esters. Examples of commonly used protecting groups for alcohols include ethers, such as methyl, methoxymethyl, methoxyethoxymethyl, methylthiomethyl, benzyloxymethyl, tetrahydropyranyl, ethoxyethyl, benzyl, 2-napthylmethyl, O-nitrobenzyl, P-nitrobenzyl, P-methoxybenzyl, 9-phenylxanthyl, trityl (including methoxy-trityls), and silyl ethers. Examples of commonly used protecting groups for sulfhydryls include many of the same protecting groups used for hydroxyls. In addition, sulfhydryls can be protected in a reduced form (e.g., as disulfides) or an oxidized form (e.g., as sulfonic acids, sulfonic esters, or sulfonic amides). Protecting groups can be chosen such that selective conditions (e.g., acidic conditions, basic conditions, catalysis by a nucleophile, catalysis by a lewis acid, or hydrogenation) are required to remove each, exclusive of other protecting groups in a molecule. The conditions required for the addition of protecting groups to amine, alcohol, sulfhydryl, and carboxyl functionalities and the conditions required for their removal are provided in detail in T. W. Green and P. G. M. Wuts, Protective Groups in Organic Synthesis (2^{nd} Ed.), John Wiley & Sons, 1991 and P. J. Kocienski, Protecting Groups, Georg Thieme Verlag, 1994. Additional synthetic details are provided below.

### Linkers

The linker component of the invention is, at its simplest, a bond between drug (A) and drug (B), but typically provides a linear, cyclic, or branched molecular skeleton having pendant groups covalently linking drug (A) to drug (B).

Thus, linking of drug (A) to drug (B) is achieved by covalent means, involving bond formation with one or more functional groups located on drug (A) and drug (B). Examples of chemically reactive functional groups which may be employed for this purpose include, without limitation, amino, hydroxyl, sulfhydryl, carboxyl, carbonyl, carbohydrate groups, vicinal diols, thioethers, 2-aminoalcohols, 2-aminothiols, guanidinyl, imidazolyl, and phenolic groups.

The covalent linking of drug (A) and drug (B) may be effected using a linker that contains reactive moieties capable of reaction with such functional groups present in drug (A) and drug (B). For example, an amine group of drug (A) may react with a carboxyl group of the linker, or an activated derivative thereof, resulting in the formation of an amide linking the two.

Examples of moieties capable of reaction with sulfhydryl groups include α-haloacetyl compounds of the type XCH₂CO— (where X═Br, Cl, or I), which show particular reactivity for sulfhydryl groups, but which can also be used to modify imidazolyl, thioether, phenol, and amino groups as described by Gurd, Methods Enzymol. 11:532 (1967). N-Maleimide derivatives are also considered selective towards sulfhydryl groups, but may additionally be useful in coupling to amino groups under certain conditions. Reagents such as 2-iminothiolane (Traut et al., Biochemistry 12:3266 (1973)), which introduce a thiol group through conversion of an amino group, may be considered as sulfhydryl reagents if linking occurs through the formation of disulfide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X═Br, Cl, or I), for example, as described by Wong Biochemistry 24:5337 (1979);

(ii) N-maleimide derivatives, which may react with amino groups either through a Michael type reaction or through acylation by addition to the ring carbonyl group, for example, as described by Smyth et al., J. Am. Chem. Soc. 82:4600 (1960) and Biochem. J. 91:589 (1964);

(iii) aryl halides such as reactive nitrohaloaromatic compounds; (iv) alkyl halides, as described, for example, by McKenzie et al., J. Protein Chem. 7:581 (1988);

(v) aldehydes and ketones capable of Schiff's base formation with amino groups, the adducts formed usually being stabilized through reduction to give a stable amine;

(vi) epoxide derivatives such as epichlorohydrin and bisoxiranes, which may react with amino, sulfhydryl, or phenolic hydroxyl groups;

(vii) chlorine-containing derivatives of s-triazines, which are very reactive towards nucleophiles such as amino, sulfhydryl, and hydroxyl groups;

(viii) aziridines based on s-triazine compounds detailed above, e.g., as described by Ross, J. Adv. Cancer Res. 2:1 (1954), which react with nucleophiles such as amino groups by ring opening;

(ix) squaric acid diethyl esters as described by Tietze, Chem. Ber. 124:1215 (1991); and

(x) α-haloalkyl ethers, which are more reactive alkylating agents than normal alkyl halides because of the activation caused by the ether oxygen atom, as described by Benneche et al., Eur. J. Med. Chem. 28:463 (1993).

Representative amino-reactive acylating agents include:

(i) isocyanates and isothiocyanates, particularly aromatic derivatives, which form stable urea and thiourea derivatives respectively;

(ii) sulfonyl chlorides, which have been described by Herzig et al., Biopolymers 2:349 (1964);

(iii) acid halides;

(iv) active esters such as nitrophenylesters or N-hydroxysuccinimidyl esters;

(v) acid anhydrides such as mixed, symmetrical, or N-carboxyanhydrides;

(vi) other useful reagents for amide bond formation, for example, as described by M. Bodansky, Principles of Peptide Synthesis, Springer-Verlag, 1984;

(vii) acylazides, e.g., wherein the azide group is generated from a preformed hydrazide derivative using sodium nitrite, as described by Wetz et al., Anal. Biochem. 58:347 (1974); and

(viii) imidoesters, which form stable amidines on reaction with amino groups, for example, as described by Hunter and Ludwig, J. Am. Chem. Soc. 84:3491 (1962).

Aldehydes and ketones may be reacted with amines to form Schiff's bases, which may advantageously be stabilized through reductive amination. Alkoxylamino moieties readily react with ketones and aldehydes to produce stable alkoxamines, for example, as described by Webb et al., in Bioconjugate Chem. 1:96 (1990).

Examples of reactive moieties capable of reaction with carboxyl groups include diazo compounds such as diazoacetate esters and diazoacetamides, which react with high specificity to generate ester groups, for example, as described by Herriot, Adv. Protein Chem. 3:169 (1947). Carboxyl modifying reagents such as carbodiimides, which react through O-acylurea formation followed by amide bond formation, may also be employed.

It will be appreciated that functional groups in drug (A) and/or drug (B) may, if desired, be converted to other functional groups prior to reaction, for example, to confer additional reactivity or selectivity. Examples of methods useful for this purpose include conversion of amines to carboxyls using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxyls using reagents such as α-haloacetates; conversion of thiols to amines using reagents such as ethylenimine or 2-bromoethylamine; conversion of carboxyls to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of drug (A) with a reactive chemical group of drug (B) without introducing additional linking material may, if desired, be used in accordance with the invention.

More commonly, however, the linker will include two or more reactive moieties, as described above, connected by a spacer element. The presence of such a spacer permits bifunctional linkers to react with specific functional groups within drug (A) and drug (B), resulting in a covalent linkage between the two. The reactive moieties in a linker may be the same (homobifunctional linker) or different (heterobifunctional linker, or, where several dissimilar reactive moieties are present, heteromultifunctional linker), providing a diversity of potential reagents that may bring about covalent attachment between drug (A) and drug (B).

Spacer elements in the linker typically consist of linear or branched chains and may include a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl.

In some instances, the linker is described by formula (II):

G¹-(Z¹)ₒ—(Y¹)ᵤ—(Z²)ₛ—(R₃₀)—(Z³)ₜ—(Y²)ᵥ—(Z⁴)ₚ-G² (II)

In formula (II), G¹ is a bond between drug (A) and the linker; G² is a bond between the linker and drug (B); Z¹, Z², Z³, and Z⁴ each, independently, is selected from O, S, and NR₃₁; R₃₁ is hydrogen, C₁₋₄ alkyl, C₂₋₄ alkenyl, C₂₋₄ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; Y¹ and Y² are each, independently, selected from carbonyl, thiocarbonyl, sulphonyl, or phosphoryl; o, p, s, t, u, and v are each, independently, 0 or 1; and R₃₀ is a C₁₋₁₀ alkyl, C₂₋₁₀ alkenyl, C₂₋₁₀ alkynyl, C₂₋₆ heterocyclyl, C₆₋₁₂ aryl, C₇₋₁₄ alkaryl, C₃₋₁₀ alkheterocyclyl, or C₁₋₁₀ heteroalkyl, or a chemical bond linking G¹-(Z¹)ₒ—(Y¹)ᵤ—(Z²)ₛ— to —(Z³)ₜ—(Y²)ᵥ—(Z⁴)ₚ-G².

Examples of homobifunctional linkers useful in the preparation of conjugates of the invention include, without limitation, diamines and diols selected from ethylenediamine, propylenediamine and hexamethylenediamine, ethylene glycol, diethylene glycol, propylene glycol, 1,4-butanediol, 1,6-hexanediol, cyclohexanediol, and polycaprolactone diol.

### Formulation of Pharmaceutical Compositions

The compositions, methods, and kits of the invention can include formulation(s) of compound(s) that, upon administration to a subject, result in a concentration of the compound(s) that treats a viral hepatitis infection. The compound(s) may be contained in any appropriate amount in any suitable carrier substance, and are generally present in an amount of 1-95% by weight of the total weight of the composition. The composition may be provided in a dosage form that is suitable for the oral, parenteral (e.g., intravenously or intramuscularly), rectal, determatological, cutaneous, nasal, vaginal, inhalant, skin (patch), ocular, intrathecal, or intracranial administration route. Thus, the composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions according to the invention or used in the methods of the invention may be formulated to release the active compound immediately upon administration or at any predetermined time or time period after administration. The latter types of compositions are generally known as controlled release formulations, which include (i) formulations that create substantially constant concentrations of the agent(s) of the invention within the body over an extended period of time; (ii) formulations that after a predetermined lag time create substantially constant concentrations of the agent(s) of the invention within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s) (sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the combination to a particular target cell type. Administration of compound(s) in the form of a controlled release formulation is especially preferred for compounds having a narrow absorption window in the gastro-intestinal tract or a relatively short biological half-life.

Any of a number of strategies can be pursued in order to obtain controlled release in which the rate of release outweighs the rate of metabolism of the compound in question._{—} In one example, controlled release is obtained by appropriate selection of various formulation parameters and ingredients, including, e.g., various types of controlled release compositions and coatings. Thus, the compound(s) are formulated with appropriate excipients into a pharmaceutical composition that, upon administration, releases the compound(s) in a controlled manner. Examples include single or multiple unit tablet or capsule compositions, oil solutions, suspensions, emulsions, microcapsules, molecular complexes, microspheres, nanoparticles, patches, and liposomes.

### Delivery of Compound(s)

It is not intended that administration of compounds be limited to a single formulation and delivery method for all compounds of a combination. The combination can be administered using separate formulations and/or delivery methods for each compound of the combination using, for example, any of the above-described formulations and methods. In one example, a first agent is delivered orally, and a second agent is delivered intravenously.

### Dosages

The dosage of a compound or a combination of compounds depends on several factors, including: the administration method, the type of viral hepatitis to be treated, the severity of the infection, whether dosage is designed to treat or prevent a viral hepatitis infection, and the age, weight, and health of the patient to be treated.

For combinations that include an anti-viral agent in addition to a synergistic pair of agents identified herein (e.g., a pair of Table 1), the recommended dosage for the anti-viral agent can be less than or equal to the recommended dose as given in the Physician's Desk Reference, 60^{th} Edition (2006).

As described above, the compound(s) in question may be administered orally in the form of tablets, capsules, elixirs or syrups, or rectally in the form of suppositories. Parenteral administration of a compound is suitably performed, for example, in the form of saline solutions or with the compound(s) incorporated into liposomes. In cases where the compound in itself is not sufficiently soluble to be dissolved, a solubilizer such as ethanol can be applied. The correct dosage of a compound can be determined by examining the efficacy of the compound in viral replication assays, as well as its toxicity in humans.

An antiviral agent is usually given by the same route of administration that is known to be effective for delivering it as a monotherapy. When used in combination therapy according to the methods of this invention, an agent of Table 2 or Table 3 is dosed in amounts and frequencies equivalent to or less than those that result in its effective monotherapeutic use.

### Additional Applications

If desired, the compounds of the invention may be employed in mechanistic assays to determine whether other combinations, or single agents, are as effective as the combinations of the invention in inhibiting a viral disease (e.g., those described herein) using assays generally known in the art. For example, candidate compounds may be tested, alone or in combination (e.g., with an agent that inhibits viral replication, such as those described herein) and applied to cells (e.g., hepatic cells such as Huh7, Huh2, Huh 8, Sk-Hep-1, Huh7 lunet, HepG2, WRL-68, FCA-1, LX-1, and LX-2). After a suitable time, viral replication or load of these cells is examined. A decrease in viral replication or viral load identifies a candidate compound or combination of agents as an effective agent for treating a viral disease.

The agents of the invention are also useful tools in elucidating mechanistic information about the biological pathways involved in viral diseases. Such information can lead to the development of new combinations or single agents for treating, preventing, or reducing a viral disease. Methods known in the art to determine biological pathways can be used to determine the pathway, or network of pathways affected by contacting cells (e.g., hepatic cells) infected with a virus with the compounds of the invention. Such methods can include, analyzing cellular constituents that are expressed or repressed after contact with the compounds of the invention as compared to untreated, positive or negative control compounds, and/or new single agents and combinations, or analyzing some other activity of the cell or virus such as an enzymatic activity, nutrient uptake, and proliferation. Cellular components analyzed can include gene transcripts, and protein expression. Suitable methods can include standard biochemistry techniques, radiolabeling the compounds of the invention (e.g., ¹⁴C or ³H labeling), and observing the compounds binding to proteins, e.g., using 2D gels, gene expression profiling. Once identified, such compounds can be used in in vivo models (e.g., knockout or transgenic mice) to further validate the tool or develop new agents or strategies to treat viral disease.

### Exemplary Candidate Compounds

Peptide Moieties

Peptides, peptide mimetics, and peptide fragments (whether natural, synthetic or chemically modified) are suitable for use in the methods of the invention. Exemplary inhibitors include compounds that reduce the amount of a target protein or RNA levels (e.g., antisense compounds, dsRNA, ribozymes) and compounds that compete with viral reproduction machinery (e.g., dominant negative proteins or polynucleotides encoding the same).

Antisense Compounds

The biological activity of any protein that increases viral replication, viral RNA or DNA replication, viral RNA translation, viral protein processing or activity, or viral packaging can be reduced through the use of an antisense compound directed to RNA encoding the target protein. Antisense compounds can be identified using standard techniques. For example, accessible regions of the target the mRNA of the target enzyme can be predicted using an RNA secondary structure folding program such as MFOLD (M. Zuker, D. H. Mathews & D. H. Turner, Algorithms and Thermodynamics for RNA Secondary Structure Prediction: A Practical Guide. In: RNA Biochemistry and Biotechnology, J. Barciszewski & B. F. C. Clark, eds., NATO ASI Series, Kluwer Academic Publishers, (1999)). Sub-optimal folds with a free energy value within 5% of the predicted most stable fold of the mRNA are predicted using a window of 200 bases within which a residue can find a complimentary base to form a base pair bond. Open regions that do not form a base pair are summed together with each suboptimal fold and areas that are predicted as open are considered more accessible to the binding to antisense nucleobase oligomers. Other methods for antisense design are described, for example, in U.S. Pat. No. 6,472,521, Antisense Nucleic Acid Drug Dev. 1997 7:439-444, Nucleic Acids Res. 28:2597-2604, 2000, and Nucleic Acids Res. 31:4989-4994, 2003.

### RNA Interference

The biological activity of a molecule involved in a viral infection or viral replication can be reduced through the use of RNA interference (RNAi), employing, e.g., a double stranded RNA (dsRNA) or small interfering RNA (siRNA) directed to the signaling molecule in question (see, e.g., Miyamoto et al., Prog. Cell Cycle Res. 5:349-360, 2003; U.S. Pat. Application Publication No. 20030157030). Methods for designing such interfering RNAs are known in the art. For example, software for designing interfering RNA is available from Oligoengine (Seattle, Wash.).

Dominant Negative Proteins

One skilled in the art would know how to make dominant negative proteins to the molecules involved in a viral infection or viral replication. Such dominant negative proteins are described, for example, in Gupta et al., J. Exp. Med., 186:473-478, 1997; Maegawa et al., J. Biol. Chem. 274:30236-30243, 1999; Woodford-Thomas et al., J. Cell Biol. 117:401-414, 1992).

The following example is intended to illustrate rather than limit the invention.

### Example 1

### HCV Replicon Assay

The HCV replicon assay enables screening of compounds with antiviral activity against HCV viral RNA replication. Huh7 cells expressing a subgenomic RNA replicon of Con1 (genotype 1b) sequence origin and expressing the reporter enzyme luciferase were obtained from ReBLikon, GmBH. In order to perform the assay, replicon cells were seeded on a 384-well plate at 4,000 cells/well in a total volume of 30 μL well. The plated cells were incubated at 37° C., 5% CO₂. Pre-diluted compounds at a 10× concentration were added to each well to achieve the desired final concentration. Plates were centrifuged at 900×g, 1 minute following the addition of compounds. Cells were incubated an additional 48 hours or 72 hours at 37° C., 5% CO₂. Plates were removed from the incubator 30 minutes to 1 hour prior to the addition of 25 μL/well of SteadyLite luciferase assay reagent from Perkin Elmer in order to equilibrate plates to room temperature. Following the addition of SteadyLite reagent, cells were allowed to incubate for 10 minutes prior to collecting data with a luminometer. Antiviral activity was quantified by the inhibition of luciferase activity.

In order to confirm that a decrease in luciferase activity correlated with inhibition of HCV replicon replication and not an increase in cell death, a counter screen was run in tandem. Huh7 parental cells which do not express HCV replicon RNA were treated similarly to the above replicon cells; briefly, cells were seeded on a 384-well plate at 4,000 cells/well as described above. Compounds were added the following day and, after a subsequent 48-hour incubation at 37° C., 5% CO₂, 15 μl/well of ATPlite (Perkin Elmer) was added after plates were equilibrated at room temperature. The ATPlite assay provides a quantitative measure of the levels of ATP in the cell cultures in each well. Higher levels of ATP correlate with greater cellular viability. Thus, a compound with antiviral activity is expected to inhibit the levels of luciferase measured by the SteadyLite assay without any or minimal effect on the ATP levels measured by the ATPlite assay.

Using the screen described above or a similar screen, we identified the agents listed in the combinations of agents listed in Table 6 and the single agents listed in Table 8.

TABLE 6
Compound pairs

| | | Synergy |
|---|---|---|
| Compound A | Compound B | Score |
| Ro 48-8071 | Sertraline Hydrochloride | 9.386 |
| Fenpropimorph | Sertraline Hydrochloride | 5.324 |
| BIBB-515 | Sertraline Hydrochloride | 3.959 |
| Clomiphene Citrate | Sertraline Hydrochloride | 2.746 |
| AY-9944 | Amorolfine Hydrochloride | 1.964 |
| Farnesol | Sertraline Hydrochloride | 1.788 |
| Colestolone | TOFA | 1.667 |
| Triparanol | Sertraline Hydrochloride | 1.639 |
| Colestolone | Simvastatin | 1.622 |
| Terconazole | Sertraline Hydrochloride | 1.407 |
| Fenpropimorph | Ezetimibe | 1.339 |
| AY-9944 | Sertraline Hydrochloride | 1.287 |
| BIBB-515 | Colestolone | 1.214 |
| AY-9944 | Fenpropimorph | 1.192 |
| Alendronate Sodium | Sertraline Hydrochloride | 1.173 |
| Clomiphene Citrate | Fenpropimorph | 1.119 |
| Clomiphene Citrate | Ro 48-8071 | 1.077 |
| Myriocin | Sertraline Hydrochloride | 1.033 |
| Amorolfine Hydrochloride | GGTI-286 | 1.024 |
| Alendronate Sodium | Colestolone | 0.969 |
| Colestolone | Fenpropimorph | 0.965 |
| Amorolfine Hydrochloride | TOFA | 0.960 |
| Amorolfine Hydrochloride | Terconazole | 0.945 |
| Amorolfine Hydrochloride | Clomiphene Citrate | 0.934 |
| BIBB-515 | TOFA | 0.914 |
| AY-9944 | Ezetimibe | 0.843 |
| Fenpropimorph | Triparanol | 0.811 |
| Colestolone | SR12813 | 0.770 |
| Colestolone | Ro 48-8071 | 0.769 |
| Clomiphene Citrate | Terconazole | 0.727 |
| Colestolone | Ezetimibe | 0.701 |
| Colestolone | GGTI-286 | 0.699 |
| GGTI-286 | Ro 48-8071 | 0.672 |

TABLE 7
Tested concentration ranges of compounds in combination pairs

| | Range tested | | Range tested |
|---|---|---|---|
| Compound A | (μM) | Compound B | (μM) |
| Ro 48-8071 | 6.616-0.0008 | Sertraline Hydrochloride | 13.35-0.1043 |
| Fenpropimorph | 14.373-0.1123 | Sertraline Hydrochloride | 13.35-0.1043 |
| BIBB-515 | 0.089-0.0007 | Sertraline Hydrochloride | 13.35-0.1043 |
| Clomiphene Citrate | 13.389-0.1046 | Sertraline Hydrochloride | 13.35-0.1043 |
| AY-9944 | 13.389-0.1046 | Amorolfine Hydrochloride | 13.391-0.1046 |
| Farnesol | 13.403-0.1047 | Sertraline Hydrochloride | 13.35-0.1043 |
| Colestolone | 13.393-0.1046 | TOFA | 26.456-0.2067 |
| Triparanol | 6.751-0.0527 | Sertraline Hydrochloride | 13.35-0.1043 |
| Colestolone | 13.393-0.1046 | Simvastatin | 22.172-0.1732 |
| Terconazole | 13.388-0.1046 | Sertraline Hydrochloride | 13.35-0.1043 |
| Fenpropimorph | 14.373-0.1123 | Ezetimibe | 13.554-0.1059 |
| AY-9944 | 13.389-0.1046 | Sertraline Hydrochloride | 13.35-0.1043 |
| BIBB-515 | 0.089-0.0007 | Colestolone | 13.393-0.1046 |
| AY-9944 | 13.389-0.1046 | Fenpropimorph | 14.373-0.1123 |
| Alendronate Sodium | 13.393-0.1046 | Sertraline Hydrochloride | 13.35-0.1043 |
| Clomiphene Citrate | 13.389-0.1046 | Fenpropimorph | 14.373-0.1123 |
| Clomiphene Citrate | 13.389-0.1046 | Ro 48-8071 | 0.088-0.000011 |
| Myriocin | 0.671-0.0052 | Sertraline Hydrochloride | 13.35-0.1043 |
| Amorolfine Hydrochloride | 13.391-0.1046 | GGTI-286 | 8.886-0.0694 |
| Alendronate Sodium | 13.393-0.1046 | Colestolone | 13.393-0.1046 |
| Colestolone | 13.393-0.1046 | Fenpropimorph | 14.373-0.1123 |
| Amorolfine Hydrochloride | 13.391-0.1046 | TOFA | 26.456-0.2067 |
| Amorolfine Hydrochloride | 13.391-0.1046 | Terconazole | 13.388-0.1046 |
| Amorolfine Hydrochloride | 13.391-0.1046 | Clomiphene Citrate | 13.389-0.1046 |
| BIBB-515 | 0.089-0.0007 | TOFA | 26.456-0.2067 |
| AY-9944 | 13.389-0.1046 | Ezetimibe | 13.554-0.1059 |
| Fenpropimorph | 14.373-0.1123 | Triparanol | 6.751-0.0527 |
| Colestolone | 13.393-0.1046 | SR 12813 | 26.788-0.2093 |
| Colestolone | 13.393-0.1046 | Ro 48-8071 | 0.088-0.000011 |
| Clomiphene | 13.389-0.1046 | Terconazole | 13.388-0.1046 |
| Colestolone | 13.393-0.1046 | Ezetimibe | 13.554-0.1059 |
| GGTI-286 | 8.886-0.0694 | Colestolone | 13.393-0.1046 |
| GGTI-286 | 8.886-0.0694 | Ro 48-8071 | 0.088-0.000011 |

For screens involving a combination of compounds, a synergy score was calculated by the formula S=log f_{X} log f_{Y}ΣI_{data}(I_{data}−/I_{Loewe}), summed over all non-single-agent concentration pairs, and where log f_{X,Y} are the natural logarithm of the dilution factors used for each single agent. This effectively calculates a volume between the measured and Loewe additive response surfaces, weighted towards high inhibition and corrected for varying dilution factors. The synergy score indicates that the combination of the two agents provides greater antiviral activity than would be expected based on the protection provided by each agent of the combination individually. The synergy scores for the combination pairs are listed in Table 6. In general, the magnitude of the synergy score indicates the strength of the synergistic interaction. For the HCV screening described in Example 1, synergy scores ranging from about 0.8 to about 1.0 indicate an additive effect of Compound A and Compound B, and scores >1.0 indicate a synergistic effect of Compound A and Compound B. The synergy scores for the compound pairs identified in our screen are listed in Table 6. The ranges of concentrations used for each compound are listed in Table 7. These data were generated following a 48-hour cell incubation.

Using a similar assay, we identified the compounds of Table 8 as capable of inhibiting viral replication as single agents. For each compound, the maximum effect and concentration required to achieve 50% inhibition (IC50) of viral replication are listed in Table 8.

TABLE 8
Single agent compounds

| Compound | Max Eff | IC50 (μM) |
|---|---|---|
| Lovastatin | 102 | 1.64 |
| Mevastatin | 101 | 0.6 |
| TOFA | 95 | 6.25 |
| Terconazole | 95 | 1.66 |
| Itavastatin, calcium salt | 92 | 2.3 |
| Triparanol | 87 | 1.55 |
| Clomiphene citrate | 86 | 1.01 |
| AY-9944 | 85 | 2.52 |
| Colestolone | 79 | 1.62 |
| GGTI-286 | 79 | 3.7 |
| Simvastatin | 71 | 13.97 |
| Ro 48-8071 | 66 | 0.06 |
| Fluvastatin | 65 | 15.81 |
| Amorolfine hydrochloride | 62 | 7.66 |
| SR 12813 | 47 | N/A |
| BIBB-515 | 46 | N/A |
| Myriocin | 33 | N/A |

### Experiment 2

### Materials and Methods

### Cell Culture and HCV Replicon

The human hepatoma cell line Huh-7¹ and Huh-luc/neo-ET cells (ReBLikon, GmbH) were maintained in Dulbecco's modified Eagle's medium (DMEM; Gibco Invitrogen) supplemented with 10% Fetal Bovine Serum (FBS, Gibco Invitrogen),1% Penicillin/Streptomycin (Gibco Invitrogen), 1% Gluta MAX-1 (Gibco Invitrogen) and 1% Non-Essential Amino Acids Solution (Gibco Invitrogen) at 37° C., 5% CO₂. Huh-luc/neo-ET cells were grown in medium additionally supplemented with 250 ug/ml Geneticin (G418, Gibco Invitrogen). These cells stably express an HCV genotype 1b subgenomic replicon encoding firefly (Photinus pyralis) luciferase, the coding sequence for ubiquitin and neomycin phosphotransferase downstream of the HCV IRES and upstream of an EMCV IRES which mediates translation of downstream viral nonstructural proteins NS3 to NS5B². For all experimental procedures Huh-luc/neo-ET and Huh-7 parental cells were seeded in DMEM without phenol red in the absence of G418 and Penicillin/Streptomycin (screening medium).

cHTS Luciferase Assay and Cell Proliferation Inhibition Assay

The combination high throughput screening procedure including plate formats is described in Blight (Blight, K. J., McKeating, J. A. & Rice, C. M. Highly permissive cell lines for subgenomic and genomic hepatitis C virus RNA replication. J Virol 76, 13001-14 (2002)).

Cells were seeded in 30 μl of screening medium at 4000 cells/well on white (Huh-luc/neo-ET cells for viral inhibition assay) or black (Huh7 cells for proliferation inhibition assay) 384-well assay plates (Matrix) and incubated overnight for approximately 20 hours. Using a MiniTrak™ Robotic Liquid Handling System (Perkin-Elmer) 1 μl of compound stock solutions (1000× concentration in DMSO unless otherwise mentioned) in an X (2-fold dilutions of compound horizontally arrayed) or Y (2-fold dilutions of compound vertically arrayed) format was transferred from master plates into 384-well clear bottom plates containing 100 μl screening medium (dilution plates) and mixed thoroughly. From each X and Y dilution plate, 3.3 μl was subsequently transferred to the 384-well assay plates for a final compound dilution of 1:1000 generating a 9×9 (81-point) dose response matrix. The cells were then incubated for 48 hours prior to measuring luciferase activity (viral inhibition) or ATP depletion (proliferation inhibition). 25 μl of SteadyLite (Perkin-Elmer) was added to the white 384-well assay plates and 15 μl of ATPLite (Perkin-Elmer) was added to the black 384-well plates which were subsequently incubated at least 5 minutes before measuring the luminescent signal. All luminescence measurements were assayed for 0.1 sec per well with an EnVision™ Xcite multilabel automatic plate reader with Enhanced Luminescence (Perkin-Elmer) and expressed as the number of relative light units (RLU) detected. Compounds were assayed in duplicate 9×9 dose matrices on each plate and DMSO-only control wells were included as negative untreated controls.

### Immunoblot Analysis

For protein expression analysis, Huh-luc/neo-ET cells were seeded in 4 ml of medium at 250,000 cells per well on 6-well plates and allowed to adhere for 6-8 hours. Stock solutions of compound were added at a 1:1000 dilution and cells were incubated in the presence of compound over 96 hours. Medium and compounds were refreshed once after an initial incubation of 48 hours. Cells were washed in phosphate-buffered saline (PBS, Invitrogen-Gibco) and lysed by the addition of 1× RIPA lysis buffer (0.5 M Tris-HCl, pH 7.4/1.5 M NaCl/2.5% deoxycholic acid/10% NP-40/10 mM EDTA, purchased from Upstate) containing Complete, Mini Protease inhibitor cocktail and PhosSTOP phosphatase inhibitor cocktail tablets (Roche) according to the manufacturer's recommendations. Cell lysates were rocked for 30 minutes at 4° C. and centrifuged at 10,000×g for 10 minutes at 4° C. The protein concentration of each extract was determined by BCA protein assay (Pierce) according to the manufacturer's protocol. Aliquots of extract containing 6, 8 or 10 μg of protein were heated at 70° C. for 10 minutes (excluding lysates for HMGCR detection to minimize protein multimerization), separated by sodium dodecyl sulfate/polyacrylamide gel electrophoresis using NuPAGE Novex precast 10% Bis-Tris gels (Invitrogen) and transferred to polyvinylidene difluoride membranes (PVDF, Invitrogen). Membranes were blocked in 1×TBS/0.1% Tween-20 (TBS-T) containing 5% non-fat milk prior to probing with the following primary antibodies overnight at 4° C. on a rocker: mouse monoclonal anti-HCV NS5A IgG1 (1:1000, Virogen), mouse monoclonal anti-HCV NS3 IgG (1:1000, Virogen), mouse monoclonal anti-GAPDH (1:10,000, Ambion) or mouse polyclonal anti-HMGCR (1:500, Novus). Membranes were washed 3×5 min in TBS-T prior to adding a peroxidase-conjugated ImmunoPure rabbit anti-mouse IgG secondary antibody (Pierce) and incubating 1 h at room temperature. Protein bands were visualized using the chemiluminescence reagents SuperSignal West Femto Maximum Sensitivity Substrate or SuperSignal West Pico Chemiluminescent Substrate (Pierce) and an Alpha Imager digital imaging system (Alpha Innotech).

### RNA Preparation and Quantitative RT-PCR

Measurement of HCV RNA levels in response to drug were carried out by first seeding Huh-luc/neo-ET cells in 100 μl of medium at 7,500 cells per well for 72 hour drug treatments and allowed to adhere overnight for approximately 20 hours. Compounds were added at a 1:1000 dilution in duplicate and added to cells in 3 separate experiments. Total RNA was harvested using an RNeasy 96-well kit (Qiagen) according to the manufacturer's protocol and quantified using the Quant-iT™ RiboGreen® RNA Reagent (Invitrogen). Purified RNA (4 μl) was added to TaqMan reactions containing 10 μl of QuantiTect Probe RT-PCR Master Mix (Qiagen) and 0.2 μL, of QuantiTect RT Mix. For each HCV-specific reaction, 1.7 μM of forward (5′-CCATAGATCACTCCCCTGTG-3′) and reverse (5′-CCGGTCGTCCTGGCAATTC-3′) primers and 0.85 μM of HCV-specific TaqMan probe (5′-FAM-CCTGGAGGCTGCACGACACTCA-3′-BHQ) were added. All 20 μl reactions were assayed in an Eppy Twin-Tec skirted PCR plate (Eppendorf) and subjected to quantitative one-step RT-PCR with an Eppendorf Realplex4 qPCR machine (Eppendorf) using the following program: 50° C. for 30 min, 95° C. for 15 min, and 40 cycles of 95° C. for 15 s followed by 60° C. for 1 min 15 s. Absolute quantification of HCV RNA copy number was determined by comparing PCR signals to a standard curve generated from dilutions of a 160 by PCR-amplified fragment of the 5′NTR of HCV. The 5′NTR fragment was generated by using the HCV-specific forward and reverse primers mentioned above and serial 10-fold dilutions were made in nuclease-free water containing yeast tRNA (25 μg/μl) as a carrier. Concentration of the 160 by HCV standard was determined by optical density spectrophotometry at 260 nm and the corresponding copy number was determined using the following formula for double-stranded DNA molecules: (g of standard ×6.023×10²³ molecules/mole)/(660 g/mol/base×length of amplified product in bases)^{3,4}. All qPCR samples quantified by comparison to the standard curve were subsequently normalized to total RNA per sample to account for variations in sample purification and preparation steps.

### Validation of Chemical Effects on Viral Protein Expression

To confirm effects on viral activity due to single agent treatments (FIG. 1b), Huh-luc/neo-ET replicon cells were treated with chemical probes for 96 hours. Cell lysates were harvested and antibodies for NS3, NS5A, and GAPDH were used to probe western transfers of proteins separated by 10% Bis-Tris SDS/PAGE. Protein bands were quantified using densitometry and amounts of HCV proteins NS3 and NS5A are shown as percentages normalized to GAPDH. Drug Concentrations (in μM): Colestolone (3.75), Simvastatin (3.25), SR 12813 (7.5), Farnesol (15.0), GGTI-286 (5.0), Squalestatin (0.63), Clomiphene (1.87); U18666A (0.1), Ro 48-8071 (0.02), Terconazole (3.75), Amorolfine (10.0), Fenpropimorph (15.0), AY-9944 (0.94), Triparanol (1.87), 2′-C-methylcytidine (10.0).

To validate the epistasis on HCV protein expression levels, S Cell lysates were harvested and antibodies for NS3, NS5A, and GAPDH were used to probe western transfers of proteins separated by 10% Bis-Tris SDS/PAGE. Protein bands were quantified using densitometry and amounts of HCV proteins NS3 and NS5A are shown as percentages normalized to GAPDH. Drug Concentrations (in μM): 2′-C-methylcytidine (10.0), U18666A x Simvastatin (0.04×2.8), Simvastatin (2.8), U18666A (0.04).

Impact of sterol pathway chemical probes on HMGCR protein expression. Huh7 cells were treated for 16 hours with each indicated chemical probe and total cell lystates were harvested. Antibodies specific for HMGCR and GAPDH were used to probe western transfers of proteins separated by 10% Bis-Tris SDS/PAGE. Drug concentrations (in μM): Colestolone (13.5), Simvastatin (11.3), SR 12813 (13.5), Farnesol (27.0), Squalestatin (9.0), U18666A (9.0), Ro 48-8071 (6.8), Terconazole (3.4), Amorolfine (13.5), AY-9944 (3.4) and Triparanol.

Impact on HCV RNA replication by chemical probes which stimulate HMGCR expression. Huh-luc/neo-ET cells were treated with each indicated chemical probe for 72 hr. Values represent averages of the % inhibition of HCV RNA from 3 separate RT-qPCR experiments±standard deviations after normalizing viral copy number to total cellular RNA.

### Chemical Reagents

Small molecule enzyme inhibitors used in this study were TOFA (CAS #54857-86-2), Colestolone (CAS #50673-97-7), SR 12813 (CAS #126411-39-0), Simvastatin (CAS #79902-63-9), Alendronate (CAS #121268-17-5), Farnesol (CAS #4602-84-0), Squalestatin (CAS #142561-96-4), Clomiphene (CAS #50-41-9), Ro 48-8071 (CAS #189197-69-1), U18666A (CAS #3039-71-2), Terconazole (CAS #67915-31-5), Amorolfine (CAS #78613-35-1), Fenpropimorph (CAS #67564-91-4), AY-9944 (CAS #366-93-8), Triparanol (CAS #78-41-1) and GGTI-286 (CAS #171744-11-9). DMSO was the solvent used for most chemical probes in this study. Dithiothreitol (DTT) at 100 mM in DMSO was used as a solvent for GGTI-286 while ddH2O was used as a solvent for squalestatin and U18666A.

### Calculations

Dose matrices were assembled from replicate combination blocks on experimental 384-well pates. Raw phenotype measurements T from each treated well were converted to normalized measures of inhibitory activity a=−log₁₀(TN) or fractional inhibition I=1−T/V relative to the median V of 20 vehicle-treated wells arranged around the plate. After normalization, we calculated average activity values between replicate measurements at the same treatment doses, along with σ₁σ₁, the accompanying standard error estimates. Single agent responses were tested at eleven serially-diluted doses and combination data as 9×9 dose matrices each testing all pairs of 8 serially-diluted single agent concentrations along with their single agent doses as a control.

The synergy for each combination was determined using a superposition of effect model (SPE), where a_{SPE}=max(aₘᵢₙ, min(aₘₐₓ, aₘᵢₙ+aₘₐₓ)), if aₘᵢₙ and aₘₐₓ are the lesser and greater single agent activities at the same concentrations as in a tested combination point. SPE represents a model of expected response for non-interacting drug targets when each drug could be either inhibitory or stimulatory. When both drugs act in the same direction, a_{SPE} at any pair of concentrations is equal to the less extreme of the single drug activities at the component concentrations. When they act in opposing directions (one stimulatory and the other inhibitory), a_{SPE} is simply the sum of the drug activities. Overall synergy for a combination was measured using a synergy score S=Σ_{doses}(a_{data}−a_{SPE}), which is the sum of the differences between the measured activity and the SPE expectation, over all combined concentrations tested. Combinations with S>0 have response surfaces that are mostly more inhibited than the SPE expectation, resulting either from synergistic activity for inhibitory agents (both with a >0), or from the inhibitor's activity dominating at high combined concentrations for drugs with opposing activities. Similarly, combinations with S<0 represent either antagonism between inhibitory agents or dominance of the stimulatory agent.

### Results

To elucidate the detailed mechanism connecting the sterol biosynthesis pathway to HCV replication, we conducted a chemical genetic screen using our cHTS platform (as described in Borisy, A. A. et al. Systematic discovery of multicomponent therapeutics. PNAS 100, 7977-82 (2003), and Lehár, J. et al. Synergistic combinations tend to improve therapeutically relevant selectivity. Nature Biotechnology 27, (in press) (2009)). We selected 16 chemical probes (Table 9) to provide dense sampling of the sterol pathway (FIG. S1) in addition to exploring upstream enzymes and the protein prenylation pathway. This study involved comprehensively testing all the probes as single agents and in pairwise combinations, using assays that model both viral replication and host viability.

TABLE 9
Chemical probes that inhibit enzymes within and outside the sterol pathway.

| | Conc. | Target | |
|---|---|---|---|
| Drug name | Range (μM) | function | Target Protein |
| TOFA | 0.02-26.5 | Fatty acid | Acetyl-CoA carboxylase (ACoAC) |
| | | synthesis | |
| Colestolone | 0.03-29.2 | Cholesterol | Sterol regulatory element binding protein |
| | | Metabolism | (SREBP) |
| SR 12813 | 0.03-29.4 | Sterol Synthesis | 3-hydroxy-3-methylglutaryl-Coenzyme |
| | | | A reductase (HMGCR) |
| Simvastatin | 0.03-29.4 | Sterol Synthesis | 3-hydroxy-3-methylglutaryl-Coenzyme |
| | | | A reductase (HMGCR) |
| Alendronate | 0.014-29.4 | Sterol Synthesis | Farnesyl pyrophosphate synthase (FPPS) |
| Farnesol | 0.03-29.4 | Sterol Synthesis | Farnesyl pyrophosphate synthase (FPPS) |
| Squalestatin | 0.03-29.4 | Sterol Synthesis | Squalene Synthase (SQLS) |
| Clomiphene | 0.03-29.4 | Sterol Synthesis | Squalene epoxidase (SQLE) |
| Ro 48-8071 | 0.03-29.4 | Sterol Synthesis | Oxidosqualene cyclase (OSC) |
| U18666A | 0.0007-10.5 | Sterol Synthesis | Oxidosqualene cyclase (OSC) |
| Terconazole | 0.03-29.4 | Sterol Synthesis | Lanosterol C14-demethylase (C14dM) |
| Amorolfine | 0.03-29.4 | Sterol Synthesis | Lanosterol C14-demethylase (C14dM)* |
| Fenpropimorph | 0.03-29.4 | Sterol Synthesis | Lanosterol C14-demethylase (C14dM)* |
| AY-9944 | 0.1-13.4 | Sterol Synthesis | Sterol delta-7 and delta-14 reductase |
| | | | (d7/d14R) |
| Triparanol | 0.03-29.4 | Sterol Synthesis | Sterol delta-24 reductase (d24R) |
| GGTI-286 | 0.07-17.9 | Protein | Protein geranylgeranyl transferase I |
| | | Prenylation | (PGGT) |

### Testing Single Agents

To confirm and extend previous studies that demonstrated modulation of HCV replication via the sterol pathway, we added chemical probes of the sterol pathway to Huh-luc/neo-ET cells expressing an HCV genotype 1b subgenomic replicon and a luciferase reporter. The resulting impact on viral replication, as measured by replicon luciferase activity, is shown as dose response curves in FIG. 1a. A host proliferation counterscreen measuring cellular ATP levels was assessed in parallel using the Huh7 parental cell line and the data were used to evaluate the selectivity of each probe. All of the compounds tested produced either increases (proviral) or decreases (antiviral) in replicon replication. We also found that Huh7 cells could tolerate a measureable reduction in ATP levels of up to approximately 30-40% without any visual impact on cell fitness, as verified by light microscopy.

Several chemical probes with selective single agent antiviral activity were identified. Colestolone, the OSC inhibitors Ro 48-8071 and U18666A, as well as amorolfine and the related compound fenpropimorph, all inhibited HCV replication with marginal impact on host cell ATP levels. Both U18666A and Ro 48-8071 were highly potent and selective HCV replication inhibitors with inhibitory concentration at 50% effect (IC50) values of 19.4 and 68.3 nM, respectively.

We validated the luciferase assay results by evaluating the impact of our chemical probes on HCV protein expression levels (FIG. 1b). Chemical probes were added to Huh-luc/neo-ET cells as described in the methods at concentrations chosen for their absence of appreciable impact on host cell ATP levels in order to avoid nonspecific antiviral effects. After a 96 hour incubation in the presence of each probe, the impact on expression of viral proteins NS3 and NS5A was normalized against the expression pattern of the housekeeping gene GAPDH using western analysis techniques.

### Combination Chemical Genetic Experiment

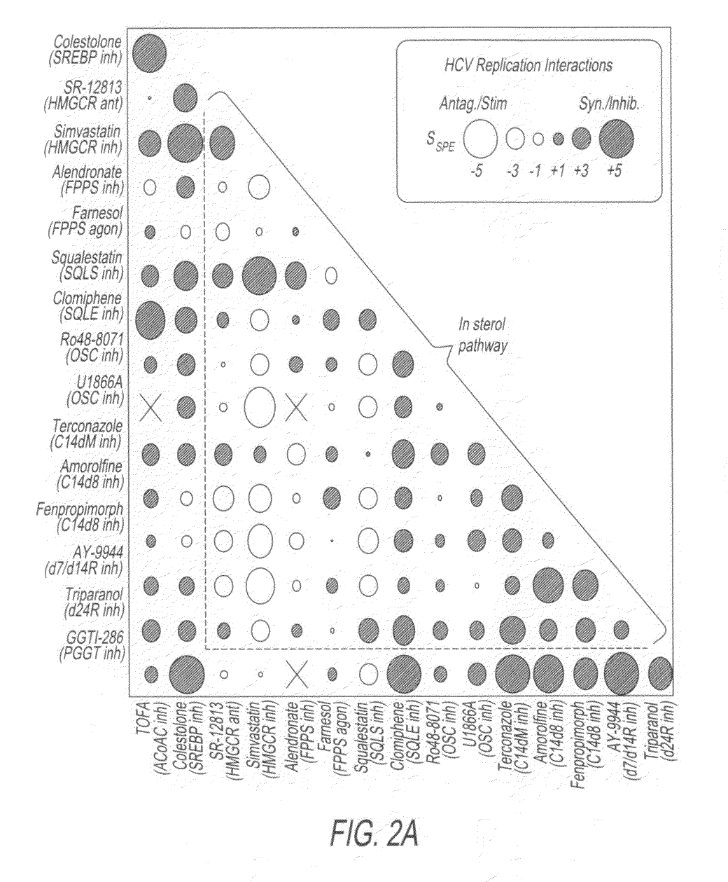

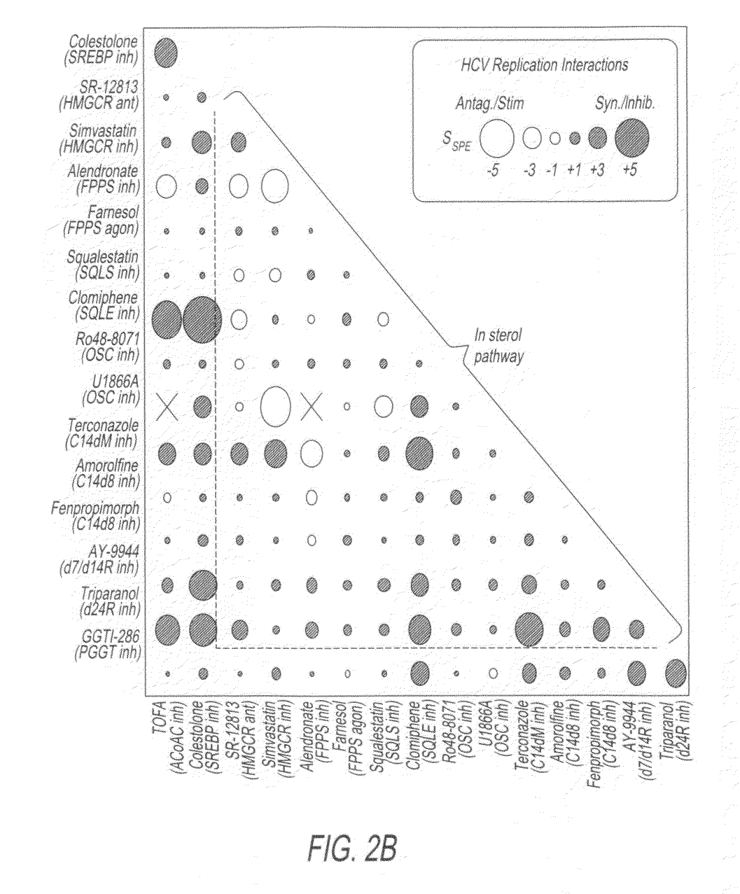

After determining single agent activities for all the chemical probes tested in this study, we designed dose-matrix experiments with concentrations centered on each drug's IC50 when possible. To evaluate the synergistic activity of compounds in combination, we compared the observed activity across the response surface to a superposition of effect (SPE) model, which smoothly interpolates between the activities of both inhibitory and stimulatory single agents. An overview of the replicon responses and the corresponding host combination activity is presented in FIG. 2.

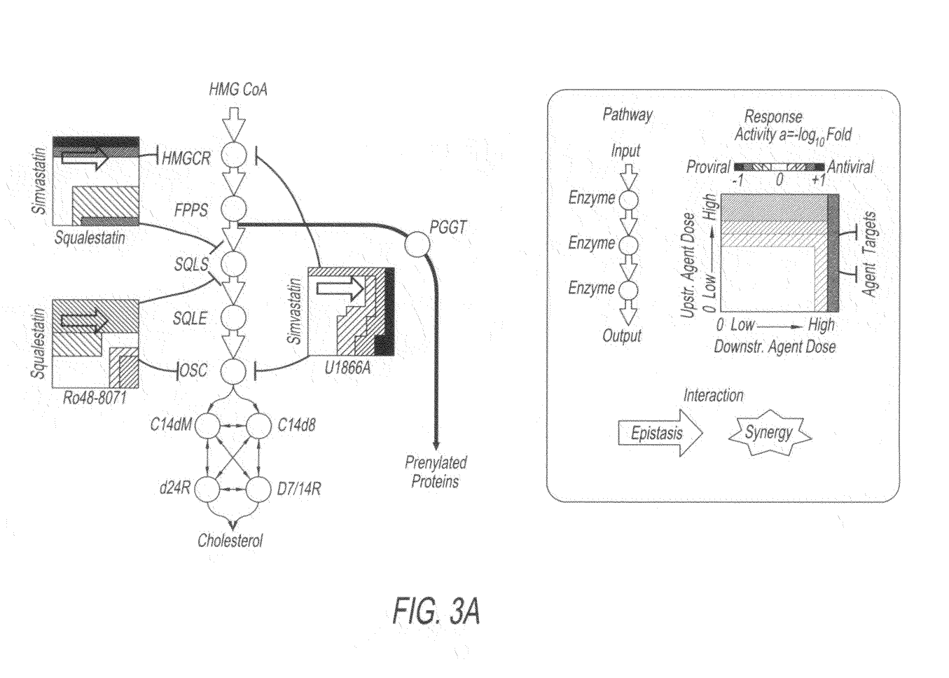

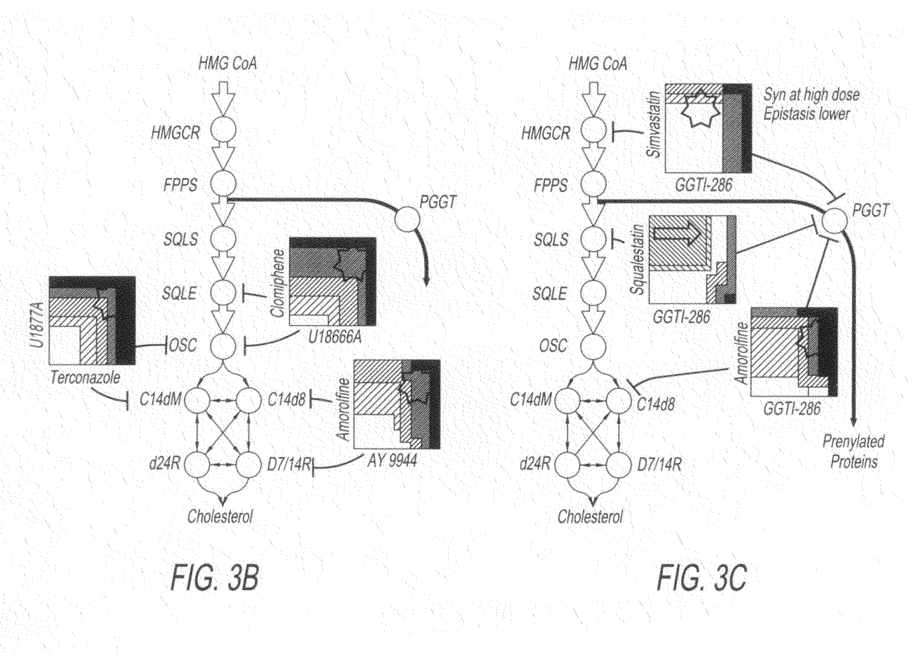

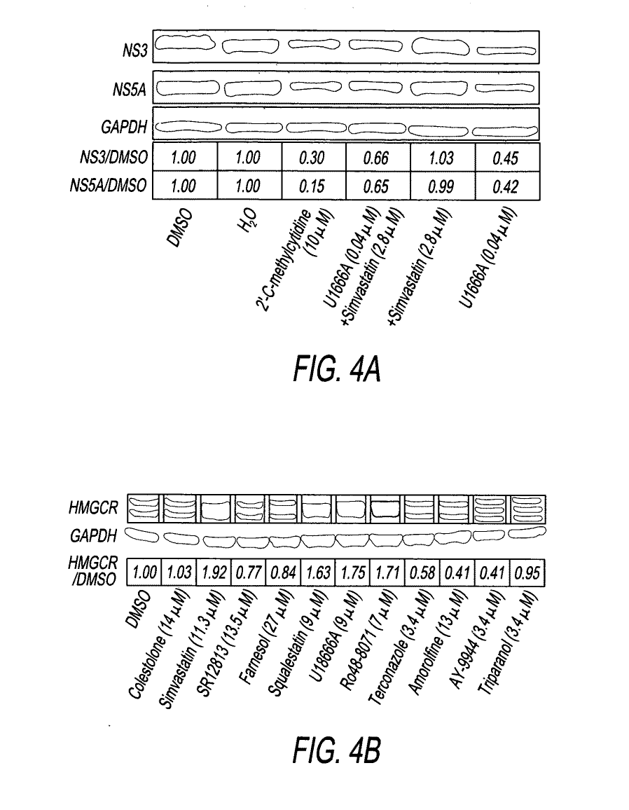

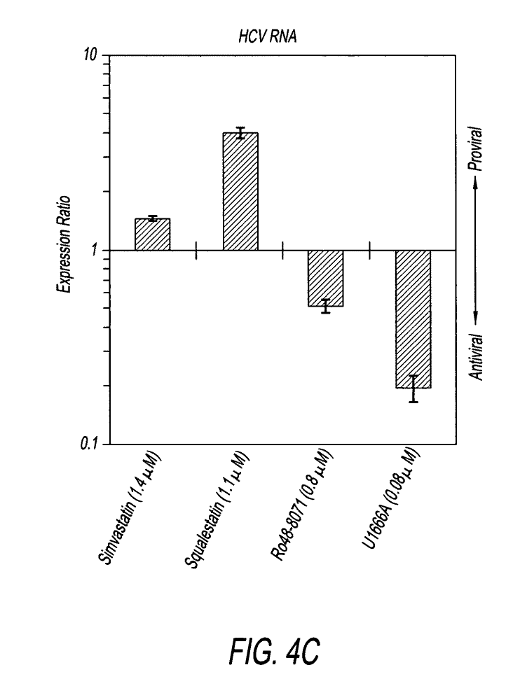

In the HCV replicon assay, however, strong mechanism-dependent patterns emerged, which are highlighted in FIG. 3. Combinations targeting enzymes upstream of squalene epoxidase (SQLE) at the top of the sterol pathway elicited epistatic responses, where the upstream agent's response predominates at high concentrations over the effect of targeting enzymes downstream of SQLE (FIG. 3a). Confirmation of simvastatin's epistasis over U 18666A was provided by western analysis (FIG. 4) and suggests that the observed contradictory effect of simvastatin is not an artifact.

Combinations targeting the downstream end of the pathway led to inhibitory synergy in both the replicon and host viability assays, especially when both agents were downstream of OSC. FIG. 3b highlights examples of antiviral synergy resulting from treatment of cells with an OSC inhibitor in combination with an inhibitor of either an enzyme upstream or downstream of OSC.

Interactions with the protein prenylation pathway also showed strong mechanistic patterns (FIG. 3c).

### Validation of Single Agent and Combination Responses Using RNA Expression

The above results suggest that HMGCR regulation may play a role in the epistasis of upstream sterol probes over downstream probes. To determine the effects of our chemical inhibitors on HMGCR expression, we treated Huh7 cells with the listed concentrations of each chemical for 16 hrs (FIG. 4b). Cell lysates were extracted according to the methods and HMGCR protein expression was analyzed by SDS-PAGE separation and western staining with antibodies specific for HMGCR and GAPDH. Treatment of cells with simvastatin or with either OSC inhibitor (U18666A or Ro48-8071) resulted in an apparent overexpression of HMGCR. None of the other chemical probes tested produced increases in HMGCR protein expression.

We next sought to evaluate the consequences of such overexpression as they pertain to HCV RNA replication. Huh-luc/neo-ET cells were treated with each inhibitor in FIG. 5 over 72 hrs in 96-well plates. Total cellular RNA was extracted from the treated cells and viral RNA copy number in each treated population was quantified by RT-qPCR and normalized to total RNA. Squalestatin and, to a lesser extent, simvastatin mediated proviral effects, consistent with the observed coincident increases in HMGCR expression. In addition, these results are also in agreement with the luciferase expression data and the viral protein expression reported above in FIG. 1.

### Other Embodiments

All publications, patent applications, including U.S. Provisional Application Nos. 60/844,463, filed Sep. 14, 2006, 60/874,061 filed Dec. 11, 2006, and 61/069,917, filed Mar. 19, 2008, titled “Compositions and Methods for Treating Viral Infections,” Attorney Docket No. 50425/009001 and U.S. patent application Ser. No. 11/900,893, filed Sep. 13, 2007, and patents mentioned in this specification are herein incorporated by reference.

Various modifications and variations of the described method and system of the invention will be apparent to those skilled in the art without departing from the scope and spirit of the invention. Although the invention has been described in connection with specific desired embodiments, it should be understood that the invention as claimed should not be unduly limited to such specific embodiments. Indeed, various modifications of the described modes for carrying out the invention that are obvious to those skilled in the fields of molecular biology, medicine, immunology, pharmacology, virology, or related fields are intended to be within the scope of the invention.

What is claimed is:

1. A composition comprising
(a) a first agent that is an inhibitor of a cholesterol biosynthetic enzyme selected from the group consisting of HMG-CoA synthase, mevalonate kinase, phosphomevalonate kinase, farnesyl transferase, geranylgeranyl transferase, farnesyl diphosphate synthase, squalene synthase, squalene monooxygenase, lanosterol synthase, lanosterol 14α-demethylase, Δ14-sterol reductase, C-4 methyl sterol oxidase, 3β-hydroxysteroid dehydrogenase, 3-ketosteroid dehydrogenase, sterol Δ8,Δ7 isomerase, sterol-C5-desaturase, sterol Δ7 reductase, and sterol Δ24 reductase; and
(b) a second agent selected from the group consisting of sertraline, an analog of sertraline, UK-416244, and an analog of UK-416244.

2. The composition of claim 1, wherein said first and second agents are present in amounts that, when administered together to a patient with a viral disease, are effective to treat said patient.

3. The composition of claim 1, wherein said inhibitor of farnesyl diphosphate synthase is selected from the group consisting of alendronate, pamidronate, risedronate, and ibandronate.

4. The composition of claim 3, wherein said inhibitor of farnesyl diphosphate synthase is alendronate.

5. The composition of claim 1, wherein said inhibitor of squalene synthase is selected from the group consisting of squalestastin and TAK-475.

6. The composition of claim 1, wherein said inhibitor of lanosterol synthase is selected from the group consisting of Ro48-8071, BIBB-515, BIBB-1464, BMX-245, and BIBX-79.

7. The composition of claim 6, wherein said inhibitor of lanosterol synthase is Ro 48-8071 or BIBB-515.

8. The composition of claim 1, wherein said inhibitor of lanosterol 14α-demethylase is selected from the group consisting of terconazole, bifonazole, butoconazole, fenticonazole, fluconazole, itraconazole, ketoconazole, miconazole, omoconazole, posaconazole, voriconazole, SKF-10497, cephalosporins, Ro 09-1470, and DIO-902.

9. The composition of claim 8, wherein said inhibitor of lanosterol 14α-demethylase is terconazole.

10. The composition of claim 1, wherein said inhibitor of Δ14-sterol reductase is fenpropimorph.

11. The composition of claim 1, wherein said inhibitor of 3β-hydroxysteroid dehydrogenase is trilostane.

12. The composition of claim 1, wherein said inhibitor of Δ14-sterol is fenpropimorph.

13. The composition of claim 1, wherein said inhibitor of sterol Δ8,Δ7 isomerase is selected from the group consisting of fenpropimorph and SR31747.

14. The composition of claim 1, wherein said inhibitor of sterol Δ7 reductase is selected from the group consisting of AY-9944 and BM-15766.

15. The composition of claim 14, wherein said inhibitor of sterol Δ7 reductase is AY-9944.

16. The composition of claim 1, wherein said inhibitor of sterol Δ24 reductase inhibitor is selected from the group consisting of triparanol and brassicasterol.

17. A composition comprising two or more agents, wherein each of said agents is an inhibitor of a cholesterol biosynthetic enzyme, and wherein said agents are present in amounts that, when administered together to a patient with a viral disease, are effective to treat said patient.

18. The composition of claim 17, wherein said two or more agents are selected from the group consisting of AY-9944 and amorolfine; colestolone and simvastatin;
BIBB-515 and colestolone; AY-9944 and fenpropimorph; clomiphene and fenpropimorph; clomiphene and Ro 48-8071; alendronate and colestolone;
colestolone and fenpropimorph; amorolfine and terconzaole; amorolfine and clomiphene; fenpropimorph and triparanol; colestolone and SR 12813; colestolone and Ro 48-8071; clomiphene and terconazole; GGTI-286 and amorolfine; GGTI-268 and colestolone; and GGTI-286 and Ro 48-8071.

19. The composition of claim 17, wherein each of said agents inhibits a different step in cholesterol biosynthesis.

20. A composition comprising:
(a) a first agent that is an inhibitor of a cholesterol biosynthetic enzyme; and
(b) a second agent that is an inhibitor of cholesterol absorption; and
wherein said agents are present in amounts that, when administered together to a patient with a viral disease, are effective to treat said patient.

21. The composition of claim 20, wherein said first agent is fenpropimorph, AY-9944, or colestolone and said second agent is ezetimibe.

22. A composition comprising:
(a) a first agent that is an inhibitor of cholesterol biosynthesis; and
(b) a second agent that is an inhibitor of sphingomyelin biosynthesis; and
wherein said first and second agents are present in amounts that, when administered together to a patient with a viral disease, are effective to treat said patient.

23. The composition of claim 22, wherein said first agent is colestolone, amorolfine, or BIBB-515 and said second agent is TOFA.

24. A composition comprising:
(a) a first agent that is an inhibitor of sphingomyelin biosynthesis; and
(b) a second agent selected from the group consisting of sertraline, an analog of sertraline, UK-416244, and an analog of UK-416244.

25. The composition of claim 24, wherein said inhibitor of sphingomyelin biosynthesis is myriocin.

26. The composition of claim 1, wherein said sertraline analog is selected from the group consisting of rac-cis-N-desmethyl sertraline, (1S,4S)-desmethyl sertraline, 1-des (methylamine)-1-oxo-2-(R,S)-hydroxy sertraline, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH2, sertraline sulfonamide ethanol, sertraline nitrile, sertraline-CME, dimethyl sertraline reverse sulfonamide, sertraline reverse sulfonamide (CH2 linker), sertraline B-ring ortho methoxy, sertraline A-ring methyl ester, sertraline A-ring ethanol, sertraline N,N-dimethylsulfonamide, sertraline A ring carboxylic acid, sertraline B-ring para-phenoxy, sertraline B-ring para-trifluoromethane, N,N-dimethyl sertraline B-Ring para-trifluoromethane, and UK-416244.

27. The composition of claim 1, wherein said first and second agents are present in amounts that, when administered together to a patient with a viral disease, are effective to treat said patient.

28. The composition of claim 27, wherein said viral disease is caused by a single stranded RNA virus, a flaviviridae virus, or a hepatic virus.

29. The composition of claim 28, wherein said viral disease is caused by a flaviviridae virus selected from the group consisting of a hepacivirus, a flavivirus, a pestivirus, or a hepatitis G virus.

30. The composition of claim 29, wherein said viral disease is caused by a flavivirus selected from the group consisting of Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, royal farm, Russian spring-summer encephalitis, Saboya, St. Louis encephalitis, Sal Vieja, San Perlita, Saumarez Reef, Sepik, Sokuluk, Spondweni, Strafford, Tembusu, Tyuleniy, Uganda S, Usutu, Wesselsbron, west Nile, Yaounde, yellow fever, and Zika.

31. The composition of claim 29, wherein said viral disease is caused by a pestivirus selected from the group consisting of bovine viral diarrhea virus, classical swine fever virus, and border disease virus.

32. The composition of claim 29, wherein said viral disease is hepatitis A, hepatitis B, hepatitis C, hepatitis D, or hepatitis E.

33. The composition of claim 1, further comprising an agent selected from the agents of Table 2 or Table 3.

34. The composition of claim 1, wherein said composition is formulated for oral administration.

35. The composition of claims 1, wherein said composition is formulated for systemic administration.

36. The composition of claim 1, wherein said composition is formulated for parenteral administration.

37. A method for treating a patient having a viral disease, said method consisting of administering to said patient a composition consisting of one or more excipients and an active agent in an amount that is effective to treat said patient, wherein said active agent is selected from the group consisting of lovastatin, mevastatin, terconazole, itavastin, clomiphene, colestolone, GGTI-286, simvastatin, Ro-48-8071, fluvastatin, amorolfine, SR12813, BIBB-515.

38. A method for treating a patient having a viral disease, said method consisting of administering to said patient a composition consisting of one or more excipients and an active agent, wherein said active agent is an inhibitor of a sphingomyelin biosynthetic enzyme, in an amount that is effective to treat said patient.

39. The method of claim 38, wherein said inhibitor is selected from the group consisting of TOFA and myriocin.

40. A method for treating a patient having a viral disease, said method comprising administering to said patient:
(a) an inhibitor of a cholesterol biosynthetic enzyme selected from the group consisting of HMG-CoA synthase, mevalonate kinase, phosphomevalonate kinase, farnesyl transferase, geranylgeranyl transferase, farnesyl diphosphate synthase, squalene synthase, squalene monooxygenase, lanosterol synthase, lanosterol 14α-demethylase, Δ14-sterol reductase, C-4 methyl sterol oxidase, 3β-hydroxysteroid dehydrogenase, 3-ketosteroid dehydrogenase, sterol Δ8,Δ7 isomerase, sterol-05-desaturase, sterol Δ7 reductase, sterol Δ24 reductase, said group excluding amorolfine; and
(b) a second agent selected from the group consisting of sertraline, an analog of sertraline, UK-416244, and an analog of UK-416244.
in amounts that together are effective to treat said patient.

41. The method of 40, wherein said inhibitor of a cholesterol biosynthetic enzyme is selected from the group consisting of Ro-48-8071, AY-9944, fenpropimorph, terconazole, BIBB-515, farnesol, triparanol, alendronate, and clomiphene.

42. A method for treating a patient having a viral disease, said method comprising administering to said patient two or more inhibitors of a cholesterol biosynthetic enzyme, in amounts that together are effective to treat said patient.

43. The method of claim 42, wherein said cholesterol biosynthesis inhibitors are selected from the group consisting of AY-9944 and amorolfine; colestolone and simvastatin; BIBB-515 and colestolone; AY-9944 and fenpropimorph; clomiphene and fenpropimorph; clomiphene and Ro 48-8071; alendronate and colestolone; colestolone and fenpropimorph; fenpropimorph and triparanol; colestolone and SR 12813; colestolone and Ro 48-8071; clomiphene and terconazole; GGTI-286 and amorolfine; GGTI-268 and colestolone; and GGTI-286 and Ro 48-8071.

44. A method for treating a patient having a viral disease, said method comprising administering to said patient:
(a) an inhibitor of a cholesterol biosynthetic enzyme; and
(b) an inhibitor of cholesterol absorption
in amounts that together are effective to treat said patient.

45. The method of claim 44, wherein said inhibitor of a cholesterol biosynthetic enzyme is AY-9944, fenpropimorph, or colestolone and said inhibitor of cholesterol absorption is ezetimibe.

46. A method for treating a patient having a viral disease, said method comprising administering to said patient a pair of agents consisting of
(a) an inhibitor of a cholesterol biosynthetic enzyme; and
(b) an inhibitor of a sphingomyelin biosynthetic enzyme
in amounts that together are effective to treat said patient.

47. The method of claim 46, wherein said inhibitor of a cholesterol biosynthetic enzyme is colestolone or BIBB-515 and said inhibitor of a sphingomyelin biosynthetic enzyme is TOFA.

48. A method for treating a patient having a viral disease, said method comprising administering to said patient a pair of agents consisting of
(a) an inhibitor of a sphingomyelin biosynthetic enzyme; and
(b) a second agent selected from the group consisting of sertraline, an analog of sertraline, UK-416244, and an analog of UK-416244.
in amounts that together are effective to treat said patient.

49. The method of claim 48, wherein said inhibitor of a sphingomyelin biosynthetic enzyme is myriocin.

50. A method for treating a patient having a viral disease, said method comprising administering to said patient three or more agents wherein
(a) the first two agents are selected from the combination pairs of Table 1; and
(b) a third agent is selected from the agents of Table 2 and Table 3,
wherein said agents are administered within 6 months of each other in amounts that together are effective to treat said patient.

51. The method of claim 40, wherein said agents are administered within 28 days of each other.

52. The method of claim 51, wherein said agents are administered within ten days of each other.

53. The method of claim 52, wherein said agents are administered within 5 days of each other.

54. The method of claim 53, wherein said agents are administered within twenty-four hours of each other.

55. The method of claim 40, wherein said viral disease is caused by a single stranded RNA virus, a flaviviridae virus, or a hepatic virus.

56. The method of claim 55, wherein said viral disease is caused by a flaviviridae virus selected from the group consisting of hepacivirus, flavivirus, a pestivirus, or hepatitis G virus.

57. The method claim 56, wherein said viral disease is caused by a flavivirus selected from the group consisting of Absettarov, Alfuy, Apoi, Aroa, Bagaza, Banzi, Bouboui, Bussuquara, Cacipacore, Carey Island, Dakar bat, Dengue 1, Dengue 2, Dengue 3, Dengue 4, Edge Hill, Entebbe bat, Gadgets Gully, Hanzalova, Hypr, Ilheus, Israel turkey meningoencephalitis, Japanese encephalitis, Jugra, Jutiapa, Kadam, Karshi, Kedougou, Kokobera, Koutango, Kumlinge, Kunjin, Kyasanur Forest disease, Langat, Louping ill, Meaban, Modoc, Montana myotis leukoencephalitis, Murray valley encephalitis, Naranjal, Negishi, Ntaya, Omsk hemorrhagic fever, Phnom-Penh bat, Powassan, Rio Bravo, Rocio, royal farm, Russian spring-summer encephalitis, Saboya, St. Louis encephalitis, Sal Vieja, San Perlita, Saumarez Reef, Sepik, Sokuluk, Spondweni, Stratford, Tembusu, Tyuleniy, Uganda S, Usutu, Wesselsbron, west Nile, Yaounde, yellow fever, and Zika.

58. The method claim 56, wherein said viral disease is caused by a pestivirus selected from the group consisting of bovine viral diarrhea virus, classical swine fever virus, and border disease virus.

59. The method of any claim 40, wherein said viral disease is viral hepatitis.

60. The method of claim 59, wherein said viral hepatitis is hepatitis A, hepatitis B, hepatitis C, hepatitis D, or hepatitis E.

61. The method claim 60, wherein said hepatitis C is hepatitis C genotype 1, 2, 3, 4, 5, or 6.

62. The method claim 61, wherein said viral disease is caused by hepatitis C virus of genotype 1a or 1b.

63. The method of claim 40, wherein said agent or agents are administered to said patient by intravenous, intramuscular, inhalation, topical, or oral administration.

64. The method of claim 40, wherein said sertraline analog is selected from the group consisting of rac-cis-N-desmethyl sertraline, (1S,4S)-desmethyl sertraline, 1-des (methylamine)-1-oxo-2-(R,S)-hydroxy, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH₂, sertraline sulfonamide ethanol, sertraline nitrile, sertraline-CME, dimethyl sertraline reverse sulfonamide, sertraline reverse sulfonamide (CH₂ linker), sertraline B-ring ortho methoxy, sertraline A-ring methyl ester, sertraline A-ring ethanol, sertraline N,N-dimethylsulfonamide, sertraline A ring carboxylic acid, sertraline B-ring para-phenoxy, sertraline B-ring para-trifluoromethane, N,N-dimethyl sertraline B-Ring para-trifluoromethane, and UK-416244.

65. The method of claim 40, wherein said patient has not been diagnosed with or does not suffer from depression, major depressive disorder, obsessive-compulsive disorder, panic disorder, posttraumatic stress disorder, social anxiety disorder, generalized anxiety disorder, or premenstrual dysphoric disorder.

66. The method of claim 40, wherein said patient has not been diagnosed with or does not suffer from hypercholesteraolemia, primary familial hypercholesterolemia (heterozygous variant), mixed hyperlipidaemia (corresponding to type IIa and IIb of the Fredrickson classification), or coronary artery disease.

67. The method of claim 40, wherein said patient has not had a myocardial infarction, a cerebrovascular event, a coronary bypass surgery, or a translumen percutaneous coronary angioplasty.

68. A kit comprising:
(a) a composition consisting of one or more excipients and an active agent, wherein said active agent is an agent selected from the group consisting of lovastatin, mevastatin, TOFA, terconazole, itavastin, triparanol, clomiphene, AY-9944, colestolone, GGTI-286, simvastatin, Ro-48-8071, fluvastatin, amorolfine, SR12813, BIBB-515, and myriocin;
(b) instructions for administering said composition to a patient having a viral disease.

69. A kit comprising:
(a) a pair of agents selected from of Ro48-8071 and sertraline or an analog thereof, fenpropimorph and sertraline or an analog thereof; BIBB-515 and sertraline or an analog thereof; clomiphene and sertraline or an analog thereof; AY-9944 and amorolfine; fanesol and sertraline; colestolone and TOFA; triparanol and sertraline; colestolone and simvastatin; terconazole and sertraline; ezetimibe and fenpropimorph; AY-9944 and sertraline; BIBB-515 and colestolone; AY-9944 and fenpropimorph; alendronate and sertraline; clomiphene and fenpropimorph; clomiphene and Ro 48-8071; myriocin and sertraline; alendronate and colestolone; colestolone and fenpropimorph; amorolfine and TOFA; amorolfine and terconazole; amorolfine and clomiphene; BIBB-515 and TOFA; AY-9944 and ezetimibe; amorolfine and ezetimibe; fenpropimorph and triparanol; colestolone and SR 12813; colestolone and Ro 48-8071; clomiphene and terconazole; colestolone and ezetimibe; GGTI-286 and amorolfine; GGTI-268 and colestolone; and GGTI-286 and Ro 48-8071; and
(b) instructions for administering said agents to a patient having a viral disease.

70. The kit of claim 69, wherein said kit comprises a composition comprising said pair of agents.

71. The kit of claim 68 or 69, wherein said viral disease is hepatitis C.

72. The kit of claim 69, wherein said sertraline analog is selected from the group consisting of rac-cis-N-desmethyl sertraline, (1S,4S)-desmethyl sertraline, 1-des (methylamine)-1-oxo-2-(R,S)-hydroxy, (1R,4R)-desmethyl sertraline, sertraline sulfonamide, sertraline (reverse) methanesulfonamide, 1R,4R sertraline enantiomer, N,N-dimethyl sertraline, nitro sertraline, sertraline aniline, sertraline iodide, sertraline sulfonamide NH₂,sertraline sulfonamide ethanol, sertraline nitrile, sertraline-CME, dimethyl sertraline reverse sulfonamide, sertraline reverse sulfonamide (CH₂ linker), sertraline B-ring ortho methoxy, sertraline A-ring methyl ester, sertraline A-ring ethanol, sertraline N,N-dimethylsulfonamide, sertraline A ring carboxylic acid, sertraline B-ring para-phenoxy, sertraline B-ring para-trifluoromethane, N,N-dimethyl sertraline B-Ring para-trifluoromethane, and UK-416244.


SEQUENCE LISTING (3 sequences)

SEQ ID NO: 1 - DNA, 20 bp, Hepatitis C virus
ccatagatcactcccctgtg

SEQ ID NO: 2 - DNA, 19 bp, Hepatitis C virus
ccggtcgtcctggcaattc

SEQ ID NO: 3 - DNA, 22 bp, Hepatitis C virus
cctggaggctgcacgacactca